(19) United States
(12) Patent Application Publication
(10) Pub. No.: US 2023/0310131 A1
(43) Pub. Date: Oct. 5, 2023

(54) PERSONAL CARE PRODUCT

(71) Applicant: TRISA HOLDING AG, Triengen (CH)

(72) Inventor: Herbert FISCHER, Reitnau (CH)

(73) Assignee: TRISA HOLDING AG, Triengen (CH)

(21) Appl. No.: 18/024,563

(22) PCT Filed: Sep. 3, 2021
(86) PCT No.: PCT/EP2021/074395

§ 371 Date: Mar. 3, 2023

(30) Foreign Application Priority Data
Sep. 3, 2020 (EP) 20194459.2

(51) Int. Cl. A61C 15/04; A46B 5/00; A46B 9/04; A46B 15/00; A46B 17/04; A61C 15/02

(52) U.S. Cl. CPC A61C 15/046; A46B 5/0095; A46B 9/04; A46B 15/0069; A46B 17/04; A46B 5/0033; A61C 15/02; A46B 2200/1066

(57) ABSTRACT

A body care product, with at least one application unit and with at least one handle unit that is connected with the application unit and includes at least one material handle body, wherein the material handle body of the handle unit and/or the application unit are/is made at least partially of a paper material.

### PRIOR ART

The invention concerns a body care product.

A body care product, in particular an oral hygiene product like an interdental brush, a flosser or a toothbrush or another oral hygiene product, with at least one application unit and with at least one handle unit that is connected with the application unit and comprises at least one material handle body, has already been proposed.

Oral hygiene products are already known from DE 20 2020 000 613 and WO 2014/169398 A1. Furthermore, oral hygiene products are also known from WO 2021/081438 A1 and DE 10 2020 100 106 A1.

The objective of the invention is in particular to provide a generic device having improved characteristics with regard to producibility, comfort and ecology. The objective is achieved according to the invention by the features of patent claim 1 while advantageous implementations and further developments of the invention may be gathered from the subclaims.

### Advantages of the Invention

The invention is based on a body care product, in particular an oral hygiene product like an interdental brush, a flosser or a toothbrush or another oral hygiene product, with at least one application unit and with at least one handle unit that is connected with the application unit and comprises at least one material handle body.

It is proposed that the material handle body of the handle unit and/or the application unit are/is made at least partially, in particular at least to a large extent, of a paper material.

Preferably the paper material is in particular made of fibers, and there are in particular different processing options. The paper material may also have been treated. The paper material may be coated, like in particular varnished, for example with a lacquer for improving a connection when connecting and/or for improving water resistance, and/or may be soaked. The paper material may in particular be present in a flat form, in a block form or in a liquid to paperboard-like form and/or consistency, wherein different ways of processing are possible depending on the respective form. When in a flat form, the paper material may be rolled, layered, pressed, laminated, fluted, embossed, folded, punched and/or grained, i. e. in particular roughened or granulated. For a connection, the paper material may be glued, wherein preparing steps are required like graining or the like, welded, like in particular by ultrasound welding, (hot or cold) sealed or laminated, etc. For a connection, special coating may be necessary on the paper material in order to create the connection. Connections may be realized with a paper material or with other substances, respectively materials, named in this document. When in block form, the paper material may be processed mechanically, for example milled, drilled or punched. When in liquid form, the paper material may be cast or injection-molded, like in particular by paper injection-molding or paper casting. Herein the paper material is in particular formed in a cavity in analogy to an injection-molding process. The cast part is then hardened and/or dried. In paper injection-molding, in particular molding to other parts, respectively other materials, is also conceivable, for example plastic overmolded with paper. Alternatively, a plastic part may also be molded to a paper material. All in all, a product structure of the body care product may be of different types, like in particular completely of a paper material or in a hybrid technique, for example of a paper material with plastic, in particular for example with an exchangeable head and/or with a handle unit made of paper and an application unit made of plastic. Further material combinations with paper may be, as mentioned above, plastic and/or metal and/or organic materials like wood, bamboo and/or inorganic materials like stone, glass etc.

An implementation of the body care product according to the invention allows providing advantageous properties with regard to ergonomics and to manufacturability of the body care product. It is in particular possible to provide an advantageously ecological body care product. In particular, advantageously simple disposal of the body care product is achievable.

The body care product is in particular implemented by an oral hygiene product. An “oral hygiene product” is in particular to mean a toothbrush and/or an interdental cleaner, in particular an interdental brush, and/or a flosser and/or a tongue cleaner and/or a toothpick. Advantageously the oral hygiene product is embodied as a toothbrush, in particular a manual toothbrush, as an interdental brush and/or as a flosser. Alternatively, the oral hygiene product may be implemented by an electrical toothbrush. Herein the oral hygiene product—if realized as a toothbrush—may in particular be a disposal toothbrush (for one-time use), a multiple-use toothbrush (for multiple use) or an exchangeable-head toothbrush. The oral hygiene product may also be implemented generally by a brush product. A “body care product” is in particular to mean an oral hygiene brush and/or a cosmetic brush and/or a hairbrush and/or a household brush. For example, manual toothbrushes like multiple-use toothbrushes, exchangeable-head toothbrushes, disposable toothbrushes or single-tuft brushes, are conceivable as oral hygiene brushes as well as electrical toothbrushes, hybrid toothbrushes, interdental cleaners, in particular with twisted-in bristles, in injection-molded implementation or as flossers, tongue cleaners and/or dental floss. For example, mascara brushes, nail polish brushes, face brushes, applicators, in particular also hair-dye applicators, massaging apparatuses, make-up brushes, shaving brushes and/or safety razors or other body care products are conceivable as body care products, in particular cosmetic brushes. For example, dishwashing brushes, floor wipers and/or brooms are conceivable as household brushes.

In the case of an implementation as a toothbrush, it is in particular conceivable that the application unit is composed of a base body and a bristle platelet, which is furnished with bristles and/or alternative cleaning elements. The application unit may furthermore be implemented of a base body which is overmolded with bristles. Preferably the body care product consists of an application unit with bristles and the handle unit, wherein the application unit in particular has a neck region that connects the application unit to the handle unit. All sub-elements may be realized of at least one hard component and/or one or several soft component/s.

The body care product in particular has a longitudinal axis, which is advantageously arranged at least substantially parallel to a main extension direction of the body care product. Preferentially the longitudinal axis runs at least section-wise within the body care product, in particular through the gravity mass center of the body care product. In particular, the longitudinal axis of the body care product is a central axis of the body care product and/or a central axis of the handle unit. By a “central axis” of an object is herein in particular an imaginary axis to be understood which runs within the object parallel to a main extension direction of the object and intersects with the object in maximally two points. “At least substantially parallel” is here in particular to mean an orientation of a direction relative to a reference direction, in particular in a plane, wherein the direction has a deviation from the reference direction that is in particular smaller than 8°, advantageously smaller than 5° and especially advantageously smaller than 2°. By a “main extension direction” of an object is herein in particular a direction to be understood which runs parallel to a longest edge of a smallest imaginary cuboid just still completely enclosing the object. A “main extension” of an object is in particular to mean, in this context, an extension of a longest edge of a smallest imaginary cuboid just still completely enclosing the object.

Advantageously the application unit comprises at least one cleaning zone, which is configured for a tooth-cleaning application, in particular in a user's oral cavity. Preferably the cleaning zone comprises at least one cleaning unit. the cleaning unit may comprise a brush head, advantageously a toothbrush head, preferably having several bristles and/or bristle bundles and/or injection-molded cleaning elements, respectively injection-molded bristles, and/or soft-elastic cleaning elements. Alternatively or additionally, the cleaning unit may also be realized as an interdental brush and/or as a single tuft (e. g. a single large bristle bundle) and/or as a bow stringed with dental floss and/or with an interdental tape, preferentially an interdental ribbon, in particular as a flosser or the like. Furthermore, the application unit advantageously comprises at least the neck and/or bracket element, which is preferentially connected, in particular directly and/or integrally connected, to the cleaning zone. “Integrally” is in particular to mean at least connected by substance-to-substance bond, for example by a connection process like a welding process, a sealing process, a gluing process, an injection-molding process and/or another process deemed expedient by someone skilled in the art, and/or advantageously formed in one piece, like for example by a production from a cast and/or by a production in a one-component or multi-component injection-molding procedure, and advantageously from a single blank. “Configured” is in particular to mean specifically designed and/or equipped. By an object being configured for a certain function is in particular to be understood that the object fulfills and/or executes said certain function in at least one application state and/or operation state.

Preferably the handle unit comprises at least one handle element, which is advantageously configured to be held with a hand. It is conceivable that the handle element is implemented at least region-wise with a waist and/or with a concave surface region and/or with a convex surface region. This advantageously allows secure holding and optimizes ergonomics. Particularly preferentially the handle element is realized in an elongate fashion, a longitudinal axis of the handle element advantageously corresponding to the longitudinal axis of the body care product. The handle element is made at least partially, in particular completely, of at least one paper material. Alternatively or additionally, the handle element and/or the handle unit may be made at least partially of a hard component and/or of one or several soft component/s. Preferably the material handle body is made at least to a large extent, in particular completely, of at least one paper material. In particular, the handle element advantageously comprises at least one thumb-gripping region and/or at least one hand-gripping region. Advantageously the thumb-gripping region is arranged on the front side of the body care product, in particular on a front side of the handle element. It is conceivable that the thumb-gripping region and/or the hand-gripping region comprise/s at least an element and/or a surface structure realized of at least one paper material, of at least one soft component and/or of at least one hard component.

The synthetic materials implemented of hard and/or soft components, which are processed via injection-molding, may in particular be categorized as follows:
Conventional materials: materials, essentially new materials, which are mostly petrol-based.
Sustainable materials: as listed below and described later on, preferably bio-based, degradable and/or recycled.
   Biobased materials: material made, in particular by more than 60%, preferably by more than 80% and particularly preferentially by 100%, of sustainable raw materials. A further optional additional feature of bio-based materials is in particular that bio-based materials are biodegradable. Preferably the materials are not based on foodstuff, like in particular corn etc.
   Bio-degradable materials: material which is biodegradable according to the usual standards. This means in particular compostability (in an industrial or non-industrial fashion). Materials made of sustainable raw materials may carry this feature.
   Recycled materials: materials produced in a recycling process, like for example Post-Consumer Recycled Materials, Post-Industrial Recycled Materials, Ocean Waste Plastic or Social Plastic.

The materials used may be recyclable materials. For recyclable materials there is advantageously an option of further recycling after use.

In the context of the present disclosure almost any hard components and soft components may be used, which will be expediently and suitably combined and/or selected by someone skilled in the art. A hard component may be, for example, styrene polymerizates like styrene acrylonitrile (SAN), polystyrene (PS), acrylonitrile butadiene styrene (ABS), styrene methyl methacrylate (SMMA), styrene butadiene (SB) or the like. Furthermore, a hard component may comprise polyolefins like polypropylene (PP), polyethylene (PE) or the like, in particular also realized as high-density polyethylene (HDPE) or low-density polyethylene (LDPE). Moreover, polyesters may be used, like for example polyethylene terephthalate (PET), in particular realized as acid-modified polyethylene terephthalate (PETA) or glycol-modified polyethylene terephthalate (PETG), polybutylene terephthalate (PBT), acid-modified polycyclohexylenedimethylene terephthalate (PCT-A), glycol-modified polycyclohexylenedimethylene terephthalate (PCT-G) or the like. Furthermore, a usage of cellulose derivatives is conceivable, like for example cellulose acetate (CA), cellulose acetate butyrate (CAB), cellulose propionate (CP), cellulose acetate phthalate (CAP), cellulose butyrate (CB) or the like. Furthermore, a hard component may comprise, for example, polyamides (PA) like PA 6.6, PA 6.10, PA 6.12 or the like, poly (methyl methacrylate) (PMMA), polycarbonate (PC), polyoxymethylene (POM), polyvinyl chloride (PVC), polyurethane (PUR), polyimide (PA) or more of that kind. In particular, polyethylene (PE) and/or polyurethane (PUR) are applicable as a hard component and/or as a soft component. In particular, a hard component has an elasticity modulus of at least 1,000 N/mm², advantageously of at least 1,300 N/mm², and/or of maximally 2,400 N/mm², advantageously of maximally 1,800 N/mm². Preferably polypropylene (PP) is used as a hard component. At least some of the materials listed as hard components may be sustainable materials. In particular materials having a cellulose content are at least partially bio-based.

Advantageously, hard components are used for stable and/or structure-bearing elements, in particular in the handle element and/or in a carrier element of the application unit and/or in a fastening unit or the like. Advantageously, it is conceivable that a hard component used and a soft component used have different colors, such that surface structures, letterings, motifs and the like can be realized by an appropriate design of base body and soft element.

Soft components may be, for example, thermoplastic styrene elastomers (TPE-S), like a styrene ethylene butadiene styrene copolymer (SEBS), a styrene butadiene styrene copolymer (SBS) or the like. Moreover, utilization of thermoplastic polyurethane elastomers (TPE-U), thermoplastic polyamide elastomers (TPE-A), thermoplastic polyolefin elastomers (TPE-O), thermoplastic polyester elastomers (TPE-E) or the like is conceivable. Beyond this, a soft component may for example comprise at least one silicone. Advantageously a soft component has a Shore A hardness of maximally 90, advantageously of maximally 50 and especially advantageously of maximally 30. Preferably at least one soft component forms together with at least one hard component, in particular in at least one two-component and/or multi-component injection-molding, advantageously by means of at least one overmolding, at least a material bond. The materials mentioned as soft components may be sustainable materials. Neither a hard component nor a soft component nor a material for injection-molded bristles form a material bond with the paper material.

If realized as a toothbrush, the body care product in particular has a front side and a rear side, which are in particular arranged facing away from each other. Preferably the cleaning zone of the application unit is arranged on the front side of the body care product. The front side is in particular a side of the body care product that is visible in a viewing direction perpendicular to the longitudinal axis of the body care product and perpendicular to the width axis of the body care product. In particular, the side of the body care product which the thumb is laid upon is referred to as the front side of the body care product. The front side is usually also the side towards which the bristle field is oriented. The rear side advantageously corresponds to a side of the body care product that is visible in a viewing direction that is opposed thereto. The side of the toothbrush that is opposed to the bristle field is referred to as the rear side of the body care product. In particular, a side situated to the left in a perpendicular view onto the front side of the body care product is referred to as the lefthand side of the body care product. A side situated to the right in a perpendicular view onto the front side of the body care product is referred to as the righthand side of the body care product. In particular, an end of the body care product at which the cleaning zone is arranged is referred to as the upper side. In particular, an end of the body care product that is situated opposite the upper side and closest to the handle region is referred to as the underside.

It is further proposed that, if the body care product is realized as a toothbrush, the at least one application unit comprises a brush head, in particular a toothbrush head, with bristles. Moreover, the cleaning zone advantageously comprises at least one bristle carrier, for example a brush head base body. Advantageously, at least some or all of the bristles are conventionally extruded bristles. Bristles may herein in particular comprise at least one hard component and/or at least one soft component. Preferably the bristles are at least partially or completely manufactured from polyamide (PA) and/or from polyester (PBT, PET, PTT), any other materials (like for example the hard components listed) being conceivable and sustainable materials listed in this document (like for example PLA) being possible as well. It is also conceivable that at least some of the bristles have a tapering and/or a variable cross section at their usage-side end. Preferably the bristles are made of a single, in particular also mixed, material. However, bristles with several components are also conceivable, which may in particular be producible and/or produced via at least one co-extrusion. The bristles may, for example, be producible and/or produced via extrusion, cutting to length and/or subsequent processing. Differently than with injection-molded bristles or rubber-elastic massaging and cleaning elements produced via injection-molding, conventional bristles are extruded, cut and inserted at the toothbrush handle by means of adapted procedures, like for example by means of the anchor-punching method or of an anchor-free method.

In particular, cylindrical or tapering bristles having a rounded cross section are possible, any other cross sections, like for example polygonal, triangular, rectangular, square, elliptic, star-shaped, trapezoid-shaped, cross-shaped, parallelogram-shaped, rhomb-shaped or any other cross sections being also conceivable. In particular, different bristles may be used in one bristle bundle, but it is also possible that different bristle bundles, in particular each with a certain bristle type, are used. Bristles and/or bristle bundles may herein be arranged regularly but also irregularly. In particular, bristles and/or bristle bundles which are arranged in groups and/or adjoining one another may, in particular alternatingly, differ with regard to at least one feature, like for example a length, a diameter, a material, a color, a material hardness, a geometry, a tapering and/or something like that. Preferably the bristles for an application in an oral hygiene region have a diameter, in particular perpendicularly to their longitudinal axis, of at least 0.075 mm and/or of maximally 0.25 mm. Advantageously the bristles have a cross section area, in particular perpendicularly to their longitudinal axis, of at least 0.002 mm² and/or of maximally 0.2 mm². In the case of bristles applied in the field of cosmetics, e. g. bristles of an additional application element, it is also possible to use thinner bristles and/or bristles having a smaller cross section, in particular bristles with a diameter, in particular perpendicularly to their longitudinal axis, of at least 0.025 mm and/or of maximally 0.2 mm, and/or with a cross section area, in particular perpendicularly to their longitudinal axis, of at least 0.001 mm² and/or of maximally 0.15 mm². In the case of tapered bristles, in particular polyester (PBT, PET, PTT) is suitable as a material, while sustainable materials are possible as well, wherein a tapering may be generated in a mechanical and/or chemical fashion. However, other materials are also conceivable. Preferably the bristles are straight in a longitudinal direction, however, bristles which are undulating and/or drilled and/or helix-shaped and/or twisted in a longitudinal direction, in particular also combinations of different bristles, are also conceivable. Furthermore, bristles having a smooth surface are conceivable but also bristles having a textured surface.

Furthermore, the bristles are processed, in particular fastened to the bristle carrier, in particular as bristle bundles, preferably at least by means of an anchor-punching method or of an anchor-free method or the like. Preferably the bristle carrier comprises a plurality of, in particular drilled and/or injection-molded, bristle receptacles, in particular holes for bristle bundles. In the case of anchor punching, it is for example conceivable that first a base body is produced via injection molding, in particular of a hard component, preferably of the brush head, wherein advantageously blind holes for bristle bundles are formed during the injection molding. However, a subsequent drilling of blind holes is of course also conceivable. Preferably then bristles, respectively bristle bundles, are folded and are fastened in respectively one blind hole by means of at least one anchor, in particular via punching in. Sling punching is also conceivable.

As has been mentioned, alternatively anchor-free methods are also conceivable, wherein advantageously bristles, respectively bristle bundles, are not folded. In this case bristles, respectively bristle bundles, have approximately half the length compared to anchor punching. For example, it is herein conceivable that the bristle bundles may first be singulated and melted and/or, in particular subsequently, their bristle ends may be overmolded for fastening or may be fixed by a material compaction of the brush head. Herein bristle bundles may advantageously be brought together.

Beyond this it is conceivable that the application unit is composed of a base body and a bristle platelet which is furnished with bristles and/or alternative cleaning elements. For this purpose, firstly bristle platelets with through-holes are produced via injection molding, and then bristles are guided through said through-holes. Preferably the bristles are subsequently connected, in particular melted, on a rear side at least bundle-wise, preferably with one another and/or with the respective bristle platelet. Bristle platelets furnished with bristles in this way may then be connected, for example welded, sealed and/or glued, and/or connected mechanically by means of riveting and/or caulking, preferably via ultrasound welding, with a base body, in particular a brush head. For this purpose, the base body, in particular the brush head, preferably has a recess which the bristle platelet may be inserted in. Alternatively, the bristle platelet may as well be set onto the surface of the base body. In this context, the Anchor-Free-Tufting method is referred to as a known production method, which in particular allows bringing bristle bundles together. In particular, a side of the bristle platelet which is laid into a recess of the base body or onto the base body and which points toward the rear side of the body care product, is referred to as the underside of the bristle platelet. Correspondingly the upper side of the bristle platelet points toward the front side of the body care product. The base body may consist of a hard component that is made of a sustainable material or of a paper material.

The bristle platelet may alternatively be produced of a paper material or of a material that is made of fibers, respectively pulp, or of wood. The bristle platelet that is made of a paper material has outside the bristle field a peripheral region that is provided with a connecting agent, like sealing wax, glue or something similar, and which enables a sealing of the bristle platelet with a handle unit or with a bade body made, for example, of a paper material or of a hard component or of a sustainable material. Herein in particular said peripheral region is connected to the handle unit, respectively the base body. Furthermore, the peripheral region of the bristle platelet may have an effective ledge, such that a recess is formed on the rear side of the bristle platelet, which ensures that the melting of the bristles does not reach beyond the edge of the bristle platelet and does not cover the peripheral region either.

A bristle platelet made of a paper material or wood is formed from flat paper or from wood panel-layouts (e. g. veneer). Possible methods are for example punching, laser cutting or cutting. The flat area of the bristle platelet has a thickness of 0.1 mm to 3.0 mm, preferably 0.3 mm to 2.0 mm, particularly preferentially 0.5 mm to 1.5 mm.

As an alternative, a bristle platelet may be realized that is produced together with the bristles/cleaning elements, and is preferably realized integrally with the bristles/cleaning elements. In this case everything can be produced in an injection-molding process. Preferentially material for injection-molded bristles is used for this purpose. In addition, a hard component and/or a soft component may be utilized. This bristle platelet may also be connected to the base body via a great variety of connection techniques as mentioned above, for example by gluing, sealing, welding, caulking, riveting.

As an alternative, the production of a bristle-furnished element without a bristle platelet may be provided. Producing this element, respectively furnishing this element with bristles, is realized in a manner similar to the production of a furnished bristle platelet. Bristles are provided in the required configuration and are melted at their ends. The melting must in this case be realized in such a way that a melt blanket is formed from the bristle component and spread over all bristle bundles and possible further elements. The bristle-furnished element is then mounted onto a brush body/base body. The element is herein not provided with a bristle platelet but is stable in itself thanks to the melt blanket. The melt blanket forms a plane, thus serving as a basis for the connection to the base body. This element may also be connected to the base body via a great variety of connection techniques as mentioned above, for example by gluing, sealing, welding, caulking, riveting. The base body may herein have a recess such that the melt blanket may be sunk and fastened therein. However, a fastening directly on the surface is also conceivable.

Instead of a bristle platelet, it is also possible that the bristles/cleaning elements are injection-molded to the base body directly. In such a case a base body that is made of a paper material would also be overmolded directly.

A further possible method for an anchor-free furnishing with bristles is a manufacturing, in particular an injection-molding, of a brush head having through holes for bristles. Bristles may then be guided through the through holes and be melted, in particular with one another and/or with the brush head, on a rear side. Preferably, an overmolding of the melted regions and/or of the brush head, in particular with at least one soft component, is implemented after that.

As an alternative to the separate bristle platelet, the brush head is formed from a paper material or from wood as a component of the base body, respectively the handle body, and/or is integrally connected with the base body, respectively the handle body. Analogously, the brush head is provided with through holes in this variant as well. The bristles may also in this case be guided through the through holes and melted on the rear side at least in bundles, in particular melted with one another and/or with the brush head. The melted regions, respectively the rear side of the brush head, may then be closed or covered with a cover element that is made of a paper material by connecting the cover element to the brush head. In this regard, once again the aforementioned connection techniques are possible.

It is moreover conceivable to manufacture first a brush head having blind holes, for example by injection-molding and/or by drilling the blind holes and/or by cutting/punching. Bristles are in this case in particular laid together to form bundles and are melted and/or otherwise connected at one end. The brush head is then heated and/or wetted with water or water vapor. Then, advantageously bristle bundles can be inserted into the blind holes on the connected side and can be anchored by pressuring and/or by compressing the brush head. In particular, herein the heated and/or wetted blind holes, respectively the brush head, are/is deformed and/or compressed, such that the bristle bundles get anchored therein. The brush head may be deformed at the same time. This method may be applied for hard components, preferably by additional moistening, but also for paper materials and/or wood.

A further possible method for furnishing the brush head with bristles is twisting-in, wherein a cylindrical brush is produced. Herein, for example, bristles respectively filaments, are fed from a reel, wherein in particular several bristle strands/filament strands are wound on a reel. For a machine loading in each case a plurality of reels will be pretensioned as each bristle/filament in the brush corresponds to one bristle strand/filament strand. Alternatively, the bristles can be taken from pre-cut bristle disks. The bristles/filaments are spread in the correct width so as to have the width in which they will be inserted into the brush. The bristles/filaments are pulled forward in such a way that they are then exposed for the next step, which means that a wire can be guided above them. After that a wire is fed to the machine from a reel, i. e. the wire is wound off and is entered into the process. The wire is cut to a length that is greater than the wound-off length of the twisted-in brush; the final cutting-to-length will be done after twisting-in. The wire is bent to form a U, such that the open side can then be slid over the bristles/filaments in order to thread the bristles in. The wire is held at the bottom of the U. Then the open wire end is clamped such that the bristles/filaments are held between the wire portions. The bristles/filaments are cut to a length that is greater than the final length in the brush, such that the brush may be cut correctly later on when the bristles/filaments have been twisted in. The wire is rotated such that the bristles/filaments are clamped between the wire and are thus fixed. When the bristles/filaments have been fixed in the wire, they are cut to the correct length and profiled. After completion of the brush portion surplus wire is cut off.

Preferably, during an injection-molding procedure, in particular a two-component and/or multi-component injection-molding, materials of injection-molded bristles do not enter into a material bond with other soft components and/or hard components and/or paper material and/or wood and/or other materials of the body care product. Preferentially injection-molded bristles are instead connected to soft components and/or hard components via a form-fit connection, for example by at least one undercut and/or at least one breakthrough at the base body or at the brush head and/or via at least one at least partial overmolding, wherein in particular an atrophy connection and/or a shrinkage connection are/is conceivable. However, a connection at least via a material bond is also conceivable, in particular with affine, respectively compatible, synthetic materials and/or components.

For any injection-molding procedures mentioned principally a one-component, two-component and/or multi-component injection molding is conceivable. Herein, as has been mentioned, materials used, in particular different soft components and/or hard components and/or material for injection-molded bristles, may be connected and/or get connected by substance-to-substance bond and/or by a form-fit connection. It is also conceivable that articulate, respectively mobile or flexible, connections are formed by means of suitable injection-molding steps. Principally, for example, hot-runner procedures, cold-runner procedures and/or co-injection-molding procedures are possible.

Alternatively or additionally to a brush head furnished with bristles, the application unit may also comprise at least one tongue cleaner and/or at least one alternative cleaning and/or massaging element. These may in each case be implemented of a soft component, of a hard component or of a combination of soft component and hard component, and/or may advantageously be producible and/or produced via injection molding.

Preferably injection-molded bristles are made at least partially, advantageously completely, of a thermoplastic polyurethane elastomer (TPE-U). Herein it is conceivable to use a modified polyurethane elastomer (TPE-U), which may be modified in particular with regard to improved flowing characteristics and/or quick solidification, in particular quick crystallization, advantageously already at rather high temperatures. Of course, other materials are also conceivable, for example thermoplastic polyester elastomers (TPE-E), thermoplastic polyamide elastomers (TPE-A), polyethylene (PE) implemented, for example, as low-density polyethylene (LDPE) or linear-low-density polyethylene (LLDPE) or the like. Materials for injection-molded bristles advantageously have a Shore D hardness of at least 0, especially advantageously of at least 30 and/or of maximally 100, advantageously maximally 80. In particular, a Shore hardness of a material of injection-molded bristles is advantageously higher than a Shore hardness of other soft components used, for example, for handle elements, massaging elements, further cleaning elements, or the like. The materials applied for a production of injection-molded bristles may be sustainable materials.

Principally a utilization of water-soluble polymers is also conceivable, for example for hard components, soft components, injection-molded bristles, tongue cleaners or other elements of the body care product.

For the hard component and/or for the soft component and/or for the material for injection-molded bristles, it is also possible to use bio-plastics, which may in particular be obtained from sustainable raw materials, which may be biodegradable, in particular compostable, and/or may be implemented of a recycled and/or recyclable material. Preferably the material is in particular implemented by a synthetic material. Preferably the biodegradable, in particular compostable, and/or recycled and/or recyclable material is implemented of a bio-plastic, in particular a plastic based on sustainable raw materials and/or of a biodegradable synthetic material. The material may therefore in particular be fossil-based and biodegradable, like for example PVOH, PCL, PBAT, PET or PBS, or may be based on sustainable raw materials and biodegradable, like for example PLA, PHA, cellophane or amylum blends, or may be based on sustainable raw materials but not biodegradable, like for example Ca, Bio-PE, Bio-PP, Bio-PA, Bio-PET. Different bio-plastics, deemed expedient by someone skilled in the art, are conceivable, like for example amylum-based bio-plastics, cellulose-based bio-plastics, polyhydroxy-alkanoates, like in particular polyhydroxybutyrate (PHB), polylactic acid (PLA), aliphatic and/or aromatic copolyesters, or further bio-plastics, like for example lignin-based bio-plastics. Preferentially the application unit and/or the handle unit may consist to a large extent of a biodegradable, in particular compostable, material and/or of a recycled material. Preferably the body care product is implemented of only one component. In particular, the body care product comprises a hard component. The material handle body of the application unit and/or handle unit may consist of the hard component at least partially. The hard component and/or the soft component and/or the material for injection-molded bristles is in particular implemented of a bio-plastic, which may in particular be obtained from sustainable raw materials. Raw materials may here in particular be corn, hemp, sugar, castor oil, palm oil, potatoes, wheat, sugar cane, caoutchouc, wood, the castor plant/castor bean and the like. Corresponding possible basic substances may be, for example, cellulose, amylum, lactic acid (PLA), glucose, chitin, chitosan or the like, from which in particular corresponding bio-plastics may be synthetized.

Furthermore, the utilization of sustainable dental floss, like in particular dental floss made of the aforementioned sustainable materials, like for example renewable materials like cellulose, organic materials like natural silk etc., is also conceivable. Beyond this the utilization of the sustainable materials mentioned is conceivable for bristles/filaments, like for example bristles/filaments made of cellulose. Moreover, the utilization of the sustainable materials mentioned for plastic films, in particular instead of or in combination with a paper material, like in particular PLA films, is also conceivable for flossers or other applications. Films are in particular used with the paper material as laminate, thus they quasi form a coating of the paper material.

It is also conceivable that separation aids and/or predetermined break points are provided at the body care product, where it is possible to disassemble the body care product for disposal, and to sever or separate the body care product with or without simple auxiliary means (like for example scissors, knife, screwdriver, etc.) so as to enable a separation of the different materials and/or components for disposal at least partially in a respectively unmixed fashion. For example, gripping lugs, e. g. between layers that are to be separated, perforations and removable projections may be realized, by means of which the materials and/or layers can be separated. Alternatively or additionally, there may be sections which are not connected, not sealed or not welded and are provided for a separation. Furthermore, auxiliary perforations may be provided, for example for a separation of brush and paper, in particular in the case of interdental brushes or flossers. Preferably, in particular separation aids are provided at the body care product, like in particular pulling tabs, gripping lugs, and/or perforations.

Removal of portions and/or elements of the body care product, and/or at least partial separation of materials (e. g. separation of the application unit from the handle portion; of the injection-molded bristles, respectively the bristle platelet, from the head portion; of the dental floss from the handle portion) is to be realized only once (e. g. for a separate disposal of materials). Preferably, re-connection of the separated elements is not intended.

By “at least to a large extent” is in particular to be understood that at least a principal component of a material of the material handle body of the handle unit and/or of the application unit is a paper material. Preferably at least 50 weight percent, preferably at least 70 weight percent and particularly preferentially at least 90 weight percent of the material handle body of the handle unit and/or of the application unit are made of a paper material. Particularly preferentially the material handle body of the handle unit and/or the application unit are/is completely made of a paper material. Furthermore, a “paper material” is in particular to mean, in this context, a material, preferably a flat material, consisting essentially of fibers, which are preferably of vegetable origin. The material is preferably created by dehydrating a fiber suspension on a sieve. The fiber mat produced is then in particular densified and dried. Preferably the material is produced from cellulose, groundwood and/or recovered paper.

The material may herein be realized in one ply or in multi-ply fashion. A multi-ply paper material consists of several plies of paper having different thicknesses and/or different characteristics and/or at least partially of different materials. Preferably several plies of paper may form a layer, wherein the body may consist of one layer or several layers. Herein outer plies may have different water-repelling characteristics than inner plies. Outer plies may have a different color than inner plies. Individual plies or all plies may be realized essentially of fresh fiber, respectively primary fiber. Individual plies or all plies may be realized essentially of secondary fiber. The paper material may have up to 15 plies, preferentially up to 12 plies and especially preferentially between 2 and 10 plies. The plies may herein be created by gluing together or pressing together, in particular without using glue.

The finished body care product may, at least in individual regions, be made of several layers of the paper material. For example, 2, 3, 4, 5 or 6 layers may be formed. The number of plies is obtained by respective multiplication.

The grammage of the paper material is in particular between 50 g/m² and 1,000 g/m², preferably between 150 g/m² and 600 g/m². According to DIN 6730, different paper material may be differentiated by their grammage. Thus a material is paper up to 225 g/mm², whereas it is referred to as paperboard from 225 g/mm² upwards. In the common language, up to 250 g/m² it is referred to as paper, from 150 g/m² to 600 g/m² as cardboard and from 500 g/m² as paperboard. For cardboard, for example, solid fiberboard or corrugated fiberboard is conceivable. There are in particular different variants of cardboard, in particular on the basis of recycled material. Preferably, in particular in the case of cardboard, particularly preferably in the case of solid fiberboard, the paper material consists of fresh fiber and/or secondary fiber. The fresh fiber, respectively primary fiber, is herein produced from new material, like for example FSC wood. The secondary fiber is in particular produced from a recycled paper, respectively recycled cardboard. Alternatively or additionally, further filling materials are conceivable, like grass for grass paper. Paper is produced from fiber materials, which are at present mostly obtained from wood as a raw material. There is in particular also the option of woodfree cellulose. However, the preferred basis of the paper material is cellulose, respectively pulp, based on wood. Possible kinds of wood used as a basis are, for example, poplar, beech, spruce, pine or eucalyptus. Alternatively, the paper material may also be obtained from grass, bamboo or papyrus. The most important fiber materials are pulp, wood materials and recycled paper material. Certain recycled paper material may be less suitable for oral hygiene use because of printing ink. In addition to the fiber material or a fiber material mixture, the material may often also contain filling materials and further additives. The paper material as a whole or at least individual plies may have a recycling portion of at least 80%, wherein the paper material as a whole or at least individual plies may contain a portion of primary fibers. Using new material, respectively primary fibers, allows keeping the properties of the paper material within a narrower range and enables, among others, improved tear strength and thus stability. Different paper materials, deemed expedient by someone skilled in the art, are conceivable. It is moreover conceivable that the paper material is primed, i. e. coated, or non-primed, and/or is structured, like for example with a 3D contour. Structuring and/or coating in particular enables improved grippability of the body care product. In particular, the structuring may be realized in the paper material itself, like for example by surface processing and/or embossing, as well as by printing. Preferably a production of the paper material is realized free of an injection-molding or extrusion process, except for fiber casting or fiber injection-molding, which is executed—in comparison to thermoplastics—at lower temperatures, respectively lower pressure. The temperature is in a range of maximally 200° C. and the pressure is between 10 bar and 40 bar, preferably between 20 bar and 35 bar. In particular, the production of the paper material does not involve a melting process of thermoplastics.

The paper material may in particular be provided in different manners deemed expedient by someone skilled in the art, like in particular as sheets, as sheet-layouts, from a reel, in a block form or as a granulate or as a base material. A block may herein be made, for example, of a cardboard material and may, for example, be configured for mechanical processing, like for example milling.

Depending on an application, the paper material may have different densities, respectively thicknesses. Possible grammages are, for example, a cardboard with 330 g/m², in particular such a cardboard joined twice, for example welded, laminated etc., for a flosser or an interdental brush. Possible densities, respectively thicknesses per layer may be 150 g/m² to 650 g/m², preferably 250 g/m² to 500 g/m², particularly preferentially 300 g/m² to 400 g/m², in a connection of several sheets or layers. In the case of oral hygiene products in which flat layers of paper materials are used (e. g. for flossers or for an interdental brush), preferentially at least two layers of a paper material are used. It is however also possible to use 2, 3, 4 or 5 paper material layers. The layers may have different grammages and different paper materials. The layers may comprise plastic films. Preferably the plastic films are covered on both sides by layers of paper materials. The layers may be created by folding a flat paper material. Two or more foldings may be created. The foldings may be sequential, respectively rolling, or may respectively change sides alternatingly (accordion-fashion).

In the case of oral hygiene products in which rolled layers of paper materials are used, like for example toothbrushes, interdental brushes, safety razors etc., stability is achieved in the rolling process substantially by the number of rolled layers, respectively rotations, such that it is for example also possible to use very thin paper (with at least 40 g/m²). For the rolling, preferably densities, respectively thicknesses, of 5 g/m² to 150 g/m², preferentially 10 g/m² to 80 g/m², particularly preferably 20 g/m² to 50 g/m² are used. The number of rolled layers, respectively rotations, is set depending on a desired reel diameter, if desired depending on an inner hollow-space diameter and a density, respectively thickness, of the paper material used. This results in 1 to 150 layers, respectively rotations, preferentially 1 to 100 layers, respectively rotations, particularly preferentially 1 to 80 layers, respectively rotations. It is also possible to use paper materials having 2, 3, 4 or 5 plies for the rolling. The plies may have different grammages and paper materials. The plies may comprise plastic films. Preferably the plastic films are on both sides covered by paper material plies. The rolling may be realized as a continuous winding with a ribbon that is made of a paper material.

The paper material may preferably be made of a wood-free coated cardboard. All in all, depending on an application, in each case at least one surface of the paper material may be coated, printed, embossed or the like. For the paper material in particular a high degree of tear strength and, at least on an application side, a high water barrier. The tear strength is between 20 N and 100 N, preferably 30 N to 60 N.

In order to check the water barrier, respectively water resistance, of paper materials, these may be subjected to an immersion test. For this purpose, the paper material is cut into a pre-defined strip having, for example, a length of approximately 100 mm and a width of approximately 30 mm. The weight of this strip is measured. Then the strip is immerged in a tempered water bath. The water bath has a temperature in the range of human body temperature, which means in a range between 36° C. and 38° C. 50 mm of the strip are immerged for a pre-defined time interval. The time interval is between 5 seconds and 180 seconds, 180 seconds being the recommended cleaning time with a toothbrush. After this, the strip is removed from the water bath and the water is tapped off. The strip is weighed directly after tapping-off. Herein the difference in weight will show the amount of water absorbed in the paper material. The differences in weight between dry and wet paper materials depend on a great variety of factors. The structure of the paper material, the coating and treatment of the paper material, the water temperature, the shape of the cutting areas etc. contribute to the values.

With 60 seconds soaking in water at a temperature of 36° C. to 38° C., the best paper materials have a water absorption of 5% to 12%, preferably of 6.5% to 9.5%, directly after soaking.

The non-paper portion in the total weight of the body care product is less than 10%, preferably less than 5%, most preferably less than 3%. The non-paper portion is, for example, composed of plastic film and/or of a dental floss thread/dental floss ribbon and/or bristles and/or injection-molded parts and/or metal parts like wire or metal anchor.

Furthermore, the paper material may in particular have been subjected to additional treatment in order to obtain additional properties. For example, a coating may be provided on a surface of the paper material that is realized as a lacquer, as a glue or as a physical and/or chemical treatment for water resistance or for upgrading.

Beyond this filling materials for the paper material are conceivable. In particular, non-soluble filling materials and soluble filling materials are conceivable. Possible filling materials are antibacterial additives, e. g. silver particles, polymers for improved water resistance like for example in the production of paper cups, abrasive agents (increasing a friction coefficient), for example for an improvement of a cleaning performance if the paper material is used in the cleaning unit, or for an improvement of a hold, respectively haptics, if the paper material is used in the handle unit, and/or magnetic particles for a magnetic paper, e. g. for holding and/or storage and/or fixing of the body care product.

Further filling materials, which dissolve during usage, may be the following effective substances, which are incorporated in a suitable carrier and then released:
a) effective agents with toothpaste-like effect: sorbitol, flavoring, hydrated silica, sodium lauryl sulfate, sodium monofluorophosphate, creatine, zinc sulfate, triclosan, glycerin, sodium saccharine, propylene glycol, disodium phosphate, aluminum oxide, trisodium phosphate, sodium fluoride, betaine, titanium dioxide, cellulose gum, tetrasodium pyrophosphate, etc.
b) effective agents with antibacterial effect: sodium bicarbonate, citric acid, phosphoric acid, sodium carbonate, potassium carbonate, sodium perborate, sodium hexametaphosphate, sodium benzoate, sodium stearate, etc.
c) effective agents for an indication of successful cleaning by coloring plaque on the tooth surface: glucose, maltodextrin, magnesium stearate, flavoring, saccharine, microcrystalline cellulose, etc.
d) effective agents providing flavoring
e) effective agents providing scent

Corresponding paper materials are already known, for example, from cardboard for drinking cups provided by the Storaenso company, which is food-compatible, waterproof, compostable and printable. Furthermore, from the Kotkamills company cardboard is known which is made waterproof by dispersion while remaining fully recyclable and compostable. Moreover, cardboard tubes are already known.

The paper material may be pre-treated, for example upgraded. By an “upgrading” is herein a surface treatment of the paper material to be understood which, for example, results in a higher quality of appearance of the paper material. An upgrading may be applied as a printing process or during or after a printing process of the paper material. An upgrading may be applied during or after a process of the paper material being mounted with other elements of the body care product. The upgrading may in each case be applied partially in one or several points or may be applied/realized on the entire surface. The upgrading allows implementing, for example, metallic effects, optical distinctions or product lettering. A suitable coating may also help with attaining a reduced water intake of the paper material. The paper material, respectively the body care product made of the paper material, may thus be also suitable for multiple use. An upgrading may, for example, be a varnishing, herein for example UV high-gloss varnish, textured paint, soft-touch lacquer or glitter varnish may be used. The upgrading may also be a flocking that is applied at least partially. The upgrading may be an embossing, for example a silver foil embossing or a hologram embossing. The upgrading may be a scented varnish. As a further possibility of pre-treatment, for example an embossing of the paper material is provided.

Preferably the material handle body is layered from sheets, from a reel or from a sheet-layout. It is also conceivable that at least two layers are pre-treated, printed or upgraded before processing (cf. in this regard the additional explanations in this document). The pre-treatment, printing and/or upgrading may apply to one layer only. For example, an embossment is conceivable as a pre-treatment intended to create a structure on the surface of the final product. Alternatively or additionally, a coating is conceivable as a pre-treatment. For example, an inner face of each layer may be configured for gluing while an outer face comprises an anti-humidity protection. Each layer may thus have two faces with different coatings. Alternatively or additionally, a printing is conceivable as a pre-treatment. It is further conceivable that the at least two layers are post-treated subsequently to production. For example, coating, printing, coating of the application unit and/or laminating are/is conceivable as a post-treatment. Preferably the application unit is connected to the material handle body directly. The application unit is in particular laid in between the layers of the material base body and is connected together with the layers of paper material, respectively is anchored in between the layers while the layers are being connected.

Foil lamination and/or hot-foil embossing may be applied. There is, among others, the possibility of producing paper materials from several plies with a film. For this purpose, mat foils, glossy foils as well as structure foils may be used. This in particular provides an increased lifetime of the surfaces. Embossing/embossed-foil printing may be used—herein in particular flat embossing or structure embossing is possible. Laminating or sealing-in may also be used for upgrading.

Furthermore, it is proposed that the material handle body of the handle unit comprises at least one layer of a paper material which is realized in a rolled-up fashion. This in particular advantageously allows producing a handle body. In particular, an advantageously stable and easily manufacturable handle body can be provided.

Preferably the layer that is made of the paper material is rolled around a defined rolling axis. The rolling axis preferably runs parallel to, in particular substantially coaxially with, a longitudinal axis of the handle unit. In particular, the layer is rolled up during production of the material handle body. In a final state, in particular after production, the layer preferably forms a roll, in particular a cylindrical roll. Preferentially, in a plane perpendicular to the longitudinal axis of the handle unit, the layer has at least section-wise, in particular completely, a spiral-shaped cross section. A largest area of the layer in particular extends around the longitudinal axis of the handle unit. It would also be conceivable that the layer has a cross section that differs from a spiral, in particular within the roll. It would for example be conceivable that the layer has within the roll, in particular in the core, a triangular cross-section shape for further stiffening.

It is moreover proposed that the at least one layer of the material handle body is rolled in a cylindrical shape. This in particular advantageously allows producing a handle body. It is in particular possible to provide a handle body that is advantageously stable and easily manufacturable. In particular, an advantageous handle shape can be provided. Preferably the at least one layer of the material handle body is rolled in a hollow-cylindrical shape. The cylindrical shape may herein in particular have a shape of a base surface that is different from a circle. For example, it would be conceivable that the cylindrical shape has an oval, a regular or irregular polygonal, e. g. a triangular, tetragonal, pentagonal, hexagonal, heptagonal, octagonal, square, rectangular, a trapezoid-shaped and/or a rhomb-shaped base surface. The cylindrical basic shape may also have concave regions/elements. Concave regions may be produced after rolling by stamping. It would also be conceivable that the material base body has several layers, which are together rolled in a cylindrical shape. A “cylindrical shape” is in particular to mean, in this context, the shape of a body with two base surfaces which are situated opposite each other and have substantially the same shape, which are connected to each other via a shell and form a body. Preferably the handle body has at least substantially the shape of a circular cylinder or of a hollow cylinder. The cylinder may also be helically twisted. “At least substantially” is in particular to mean, in this context, that a deviation from a given value is in particular less than 25%, preferably less than 10% and particularly preferentially less than 5% of the given value.

Herein in particular different implementations of the body care product, deemed expedient by someone skilled in the art, are conceivable. The material handle body may in particular be produced from one or several sheets of a paper material and/or rolled from reels and/or as a roll of strips, respectively of a ribbon. The layers of the material handle body may be interconnected, for example, by gluing, sealing, welding and/or laminating. It is further conceivable that the at least one layer is pre-treated before processing. As a pre-treatment is for example an at least partial embossing of the paper material conceivable, aiming at creating a structure on the surface of the final product and/or a hollow space in the handle body. Alternatively or additionally, coating is conceivable as a pre-treatment of the paper material. For example, an inner face of each layer could be configured for gluing while an outer face has a protection against humidity. Each layer may thus have two sides with different treatments, respectively coatings. Alternatively or additionally, printing, respectively upgrading, is conceivable as a pre-treatment. It is furthermore conceivable that the at least one layer is subjected to post-treatment after production. For example, coating, printing and/or laminating are/is conceivable as a post-treatment. Alternatively or additionally, for example for a gripping geometry, re-forming and/or embossing, like in particular via pressure and heat, is conceivable as a pre-treatment.

Preferably the paper material may be rolled to form a massive roll, respectively a cylinder, or may be rolled to form a hollow roll. In the case of a hollow roll, it would in particular also be conceivable that the roll is additionally filled at least partially with a filling material, like for example a bulk material and/or a core, made of a paper material or of a further material. For the production of a hollow roll, in particular a hollow material handle body, paper material is in particular wound off reels as paper ribbons, which are together formed into a tube and/or into a hollow cylinder as has been defined above. The paper ribbons are herein in particular rolled on a core and are then connected on the core. The method herein substantially corresponds to the method for manufacturing paper drinking-straws. The application unit, in particular comprising a plastic head and/or an interdental brush, is in particular produced having an interface with the hollow tube and can be mounted directly on the tube of the material handle body. Herein it would in particular also be conceivable that the application unit is realized as an exchangeable head, the interface being integrated in the application unit between a connection section with the material handle body and the cleaning unit. There is in particular one difficulty regarding how to achieve sufficient stability as well as a stable mounting, capable of absorbing the usage forces of the body care product. Preferably, however, in an implementation of the application unit as an exchangeable head, it is in particular possible to provide a handle unit with different application units. Moreover, a tube made of a paper material in particular constitutes a simple standard product and is thus producible in an advantageously cost-efficient manner.

The material handle body may in particular be rolled in a straight or in a spiral-shaped fashion. Different ribbons may herein in particular have different characteristics, like for example different colors, so as to achieve a spirally appearance. The material handle body of the body care product in particular has a (largest) diameter of 1 mm to 25 mm, preferably of 1.5 mm to 20 mm, particularly preferentially of 2 mm to 18 mm. For an interdental brush or a mascara brush the (largest) diameter is 1 mm to 6 mm, preferably 1.5 mm to 5 mm, particularly preferentially 2 mm to 4 mm. For a toothbrush or a safety razor the (largest) diameter is 5 mm to 20 mm, preferably 7 mm to 17 mm, particularly preferentially 10 mm to 15 mm. Furthermore, in addition to the tube made of a paper material, the material handle body may optionally be provided with a plastic head, respectively with a plastic pin, which for example prevents or at least reduces water entering into the paper material and/or into a hollow space within the paper material layers. For this purpose, above all the front face of the paper material as well as hollow spaces should be especially protected. The plastic head, respectively a plastic pin, can be injection-molded non-releasably to the rolled paper material by injection molding. Furthermore, it may be provided to make the distance between the end of the handle body and the last element of the twisted-in brush (i. e. the rearmost bristle) larger than with customary interdental brushes so as to reduce moistening of the paper material. This distance may be in the range of 3 mm to 8 mm, preferably of 3 mm to 5 mm.

Beyond this, in particular an implementation of the body care product as a toothpick or interdental cleaner or dental stick is conceivable. The body care product may herein in particular be implemented of a tube or rod made of a paper material, which is at least on one side mechanically processed, respectively formed, for example cut obliquely and/or pointed, and thus forms a toothpick. For a manufacture of the body care product, herein for example paper ribbons are in particular wound off reels and are together formed to a tube (hollow) or a rod (not hollow). The paper ribbons are herein in particular rolled onto a core and are then connected thereon. After this one end of the tube may be cut obliquely and/or pointed, thus implementing the application unit at one end. It is also possible to apply other methods, described in the present document, in order to form a roll or a cylinder.

The application unit is herein in particular realized integrally with the handle unit. Optionally it would be conceivable that at least a portion of the surface of the body care product is subjected to post-treatment. Possible methods are printing, immerging, spraying etc. It would herein be conceivable that the body care product is varnished for reducing water intake or that the body care product is printed. Advantageous water resistance is herein in particular expedient as the application unit is made of paper. It is also possible to apply body care products. Of course, other methods, described in this document, may be used for upgrading, for applying filling material or for treatment of the paper material.

The post-treatment may also comprise the application of body care products, like toothpaste, onto the application unit. Furthermore, a surface of the application unit that is optimized for cleaning can be created by post-treatment, for example by applying an abrasive material. For a fixing of abrasive material, glues suitable for oral hygiene will be used.

The post-treatment may in particular be realized by immerging the respective part of the product in a fluid.

It is further proposed that the material handle body of the handle unit comprises at least one layer made of a paper material, said layer having an embossment and/or a deformation. During embossing the paper material is at least partially deformed by pressure. As a result, a three-dimensional design and/or surface structure is achievable on the surface of the paper material in portions of the surface. It is also possible to obtain hollow spaces, respectively recesses, within the paper material layers by means of embossing. This enables improved grippability and/or slip resistance of the handle body, thus facilitating a handling of the oral hygiene product. Principally embossing is possible for any paper material, be it in a flat, rolled, laminated, sealed and/or welded state. An embossing with an embossing tool (negative mold) may be aided by pressure and/or heat and/or water (vapor). An embossing may be realized in the flat state when the paper material is punched out. However, it is also possible that an embossing is carried out on a paper material that is already rolled up. The embossing may also be a kind of deep-drawing, which also provides the design options mentioned above. Deep-drawing of the paper material with a deep-drawing tool may also be aided by pressure and/or heat and/or water (vapor). Embossing or deep-drawing may also provide the paper material with a shape that brings about stability, for example a deepening in the longitudinal direction of the handle portion of a toothbrush, which makes the body more stable. Furthermore, specifically chosen coatings, like varnishes etc., may influence as well as improve dimensional stability and stability in general.

It is also proposed that the material body of the handle unit comprises at least two layers of a paper material, which are connected to each other in a layered fashion. For this, individual layers may be prepared as separate elements and/or this may be brought about by a bending of layers. Advantageously, this in particular allows producing a handle body. It is in particular possible to provide an advantageously stable handle body that is easily manufacturable. In particular, an advantageous handle shape can be provided. Preferably the material handle body, in particular the entire handle unit, consists of the at least two layers. Preferably the at least two layers are at least partially arranged above one another and connected to one another. Preferably, 2 to 6, preferably 2 to 4, layers are provided. However, 7 to 15, preferably 8 to 12, layers may be provided. The layer thickness depends on the number of plies; hence the number of layers also depends thereon for an optimum implementation of stability. The layers may be made of different paper materials, of different densities, respectively grammages, of paper materials. The paper material/s may comprise additional layers made of films, in particular plastic films. Preferentially the at least two layers are, perpendicularly to a main extension plane of the layers, at least partially arranged one above the other one and connected to one another. The at least two layers in particular adjoin each other with a largest area. The layers may be connected to each other, for example by gluing and/or sealing and/or pressing and/or welding and/or laminating. Herein pressure and/or heat and/or water (vapor) are/is applied. The layers are connected to each other in particular during production of the body care product. By a “main extension plane” of an object is in particular a plane to be understood which is parallel to a largest side surface of a smallest imaginary cuboid just still enclosing the object, and which extends through the center point of the cuboid. While the layers are being connected, the application unit may be fixed between the layers.

The connection, respectively sealing, of layers may be realized while applying different parameters. The parameters preferred for a sealing of layers, and for example of the dental floss thread for producing a flosser or attaching the twisted-in brush for an interdental brush, are listed in the following. The temperature is in a range from 150° C. to 210° C. The pressure is between 4,000 N and 6,000 N, preferably between 4,400 N and 5,100 N. The sealing time is between 0.5 seconds and 5 seconds, preferably between 1.5 seconds and 3.5 seconds.

In a sealing, it is furthermore possible that the sealing stamp is not planar. Herein a continuous or non-continuous surface and/or a structure of the stamp may be provided. Such a construction allows creating, in an interaction with the counter-surface, a sealing structure that is sealed in a differently distinct/embossed manner. There may also be a pressure progression over the sealing surface, the pressure varying over different regions of the material handle body of the handle unit.

When sealing several layers, a sealing sequence may be defined. For example, not all the layers are sealed with one another at the same time. If for example three layers are sealed, the first two layers may be sealed first and then the third layer is sealed to the two other layers. If there are four layers, for example the first two layers may be sealed and then either respectively one layer in two steps or the further two layers in one go. Step-wise sealing allows achieving optimum sealing of the individual layers. Step-wise sealing moreover allows adapting the sealing parameters like temperature, pressure, time, for each sealing individually.

Preferably the material handle body is layered from the sheet, roll or sheet-layout. It is also conceivable that before processing the at least two layers are pre-treated, embossed, printed or upgraded (cf. for this the further explanations in this document). The pre-treatment, like for example embossing, printing, upgrading, may as well apply for only one layer. For example, an embossment is also conceivable as a pre-treatment, aiming at creating a structure and/or a reinforcement crimp on the surface of the final product. Furthermore, as has been described, hollow spaces may be created between the paper material layers by an embossing of the paper material. Herein, for example, if there are two layers in a same position of the handle body, two opposed embossments are provided before connecting the paper material layers, so as to create a handle body that is at least in portions three-dimensional and not planar.

As discussed before, embossing of the handle body is also possible after connecting the layers, respectively after fixing the dental-floss thread for producing a flosser or fixing the metal wire of a twisted-in brush between the layers. In this way it is also possible to apply surface structures or a crimp for reinforcement. A hollow space can then not be created in this way. The deformation is then respectively generated so as to be convex on one side and concave on the other side.

Alternatively or additionally, coating is conceivable as a pre-treatment. For example, an inner face of each layer of the flat paper material may be configured for gluing while an outer face has a protection against humidity. Each layer may thus have two sides with different coatings, respectively properties. Alternatively or additionally, printing is conceivable as a pre-treatment. It is further conceivable that the at least two layers of the flat paper material are post-treated after being connected. For example, coating, printing, coating of the application unit and/or laminating are/is conceivable as a post-treatment. Preferably the application unit is directly connected to the material handle body. The application unit is in particular laid between the layers of the material base body and is connected together with the paper material layers. Herein, for example, welding, sealing, laminating, gluing or other connecting techniques are possible. The application unit is in particular quasi clamped between the layers that are to be connected. The application unit may herein be realized as a twisted-in brush, as a dental floss, as a tape, as a film, as a toothpick or as another body care product. When the two flat paper material layers are connected, the overlapping portion of the application unit at least partially displaces the paper material. This will reinforce a hold. The overlapping portion of the application unit may be deformed so as to increase a hold in the paper material. As has been described above, a structure and/or a crimp may be created by embossing in this part as well so as to increase the hold of the application unit.

In particular, a flosser would be conceivable as a body care product with a corresponding material base body. A dental floss may herein in particular laid into at least partially pre-punched sheets of the layers. In a production procedure, in particular sheets or sheet-layouts of paper material, are pre-punched which form the layers. A pre-punching is in particular carried out in the region where the functional element is arranged. It is also possible to create sheet-layouts by pre-punching. In particular, pre-punching of such regions of the material base body is carried out which can be punched no longer after a mounting of the functional element. After this, in particular the pre-punched sheet or sheet-layout of paper material is laid out and a thread, a ribbon or a film is inlaid. A film is thin and may be implemented of a stable, tear-resistant paper material or of a plastic, in particular a hard or soft component, or of a sustainable plastic material. A thread is preferably made of a hard component (e. g. polyamide, polyester) or of a sustainable plastic material or of Teflon. Following this, a further pre-punched sheet or sheet-layout of paper material is laid thereon and the sheets or sheet layouts are connected, for example by laminating, sealing, welding and/or gluing. Alternatively, as has been described above, the sheet or the sheet layout may be folded in a defined point in order to create the further layer, and may then be connected. After this, the body care product, in particular its outer geometry, may be punched to create individual flossers or sheet layouts of flossers, and surplus thread, ribbon or film can be removed.

It would also be conceivable that such a step is dispensed with and, instead of pre-punching, punching is carried out directly and the thread, the ribbon or the film does not protrude beyond the final outer geometry. The thread, the ribbon or the film may be anchored in different manners deemed expedient by someone skilled in the art. Differently than with flossers made of plastic, in which the thread/ribbon is fixed by overmolding, an anchoring in the paper material is more critical with regard to tear-out when in use. An anchoring may be realized, for example, by fastening the thread, the ribbon or the film by means of glue. Alternatively or additionally, an anchoring may be realized by welding. The thread, the ribbon or the film can be fastened between the layers by welding. Alternatively or additionally, an anchoring may be realized via slitted arms at the material handle body and a thread or ribbon laid therein. For this in particular a multi-ply structure is provided, a slitting being made in the middle ply, such that the thread or the ribbon adjoins the layers everywhere in the anchoring zone. The thread or the ribbon is then guided through the slitting. In addition, in particular several slits may be provided, wherein if there are several slits, the thread or the ribbon is guided through the slits alternatingly, in the way of weaving. Alternatively or additionally, an anchoring may be realized by ears at the material base body which can be folded down or bent. For this, the material base body has arms which in each case have a lateral bulge, the lateral bulges being opposed to each other. For an anchoring, the thread or the ribbon may be guided over the bulge, each thread end, respectively ribbon end, lying on a bulge. After this the thread or the ribbon is welded and the bulges are folded, in particular in such a way that the thread or the ribbon is bent and is guided round a corner. This in particular results in advantageous stabilization. Following this, in particular a further layer is applied on each side, such that the thread or the ribbon adjoins respectively one further layer in the anchoring zone.

Alternatively or additionally, an anchoring of the thread, the ribbon or the film may be realized by deformation of the paper material so as to create stabilizing structures. For example, a rake-like structure may be inserted in the layers. The deformation may in particular be realized before or after connecting. Alternatively or additionally, an anchoring can be realized by creating a structured surface for a fastening of the application unit. For example, fluted surfaces and/or rough surfaces and/or a crimp may be created by welding. Alternatively, a respective anchoring is achievable by caulking or clawing.

The thread or the ribbon may also be tied to elements at the paper material. Herein the thread or the ribbon may be guided around the element and may thus be held at the body like a lasso or a noose, the noose being tightly knotted. This may in particular be realized in one layer, wherein this layer is then on both sides covered with at least one further layer.

The pull-out weight of the thread or ribbon is at least 10 N, measured perpendicularly to the length extent of the thread/the ribbon. Preferably it is more than 20 N, particularly preferentially more than 25 N.

In particular, an interdental brush would be conceivable as a body care product with a corresponding material base body. For this purpose, an interdental brush application unit, for example realized as a twisted-in brush, may be provided for a production in a first step. After this or parallel thereto, a flat paper material may be provided which is realized as a paper sheet or paper sheet-layout. Then the interdental brush application unit is laid with its wire-side onto the flat paper material, respectively paper sheet or sheet-layout, which forms at least a first layer, and after this a further paper sheet, which forms the second layer, is laid onto the first paper sheet. Here it is also possible for the paper sheet to be folded in order to form the second layer. Following this, the layers are connected, for example welded, sealed, laminated or glued, and the material handle body is punched out and/or, in case of a sheet layout with groups of products, partly punched out and partly perforated. Principally the method is the same as with the flosser production, which was already described in detail. Instead of a thread/ribbon, the interdental brush application unit is laid with its wire-side between the layers of the paper material.

In particular, a toothbrush would be conceivable as a body care product with a corresponding material base body, which is in particular realized of connected layers with a bristle field laid in-between. For this purpose, an element may be provided which is furnished with bristles but has no bristle platelet. Furthermore, two elements of the future handle unit are prepared: on the one hand a layer that will form the front side of the body care product later on. This may be designed with different curvatures so as to give stability to the handle unit later. Furthermore, in the head region of this layer a through hole is designed which is configured for accommodating the bristle-furnished element later on, respectively the bristle-furnished element is guided through the through hole. The second layer that is prepared has essentially the same outer shape as the first layer, but does not have a recess. In the head region of the body care product this layer is implemented such that it adjoins the melt blanket of the bristle-furnished element in the mounted state. The head region of the two layers may be implemented planar or slightly curved or bent only at the edges. A limiting factor is that the bristle field is meant to hold, respectively can be fastened. In the further regions the second layer may have the same shape as the first layer, such that they adjoin each other directly; or the second layer may have a curvature inverse to the curvature of the first layer so as to form a hollow space between the layers in the mounted state. Herein, in the region of the hollow space, a connection of the layers is only possible in a peripheral region of the hollow space.

Beyond this it is proposed that the application unit displaces in at least one connection section at least one of the layers made of a flat paper material. In an anchoring of the wire of a twisted-in brush, respectively of the thread/ribbon of a flosser, a flat paper material is used which is at least partially displaceable during the anchoring, such that no or only a minimal elevation can be seen or felt in the anchoring zone. Preferably the elevation caused by this on the outer surface of the body care product in the anchoring zone is smaller than 0.2 mm, preferably smaller than 0.1 mm, particularly preferentially smaller than 0.005 mm.

For the same reason, the total thickness of the layers (there may be several) of flat paper material is chosen such that they are at least partially displaceable at least in the anchoring zone. The total thickness of the layers of flat paper material is therefore greater than the diameter of the twisted-in wire of a twisted-in brush that is to be anchored, respectively than the diameter of the thread, respectively the thickness of the ribbon, of a flosser. The ratio is in the case of a twisted-in brush at least 1.2, preferably at least 2, particularly preferentially at least 3, and in the case of a flosser at least 10, preferably at least 15, particularly preferentially at least 20.

The products, in particular if implemented as an interdental brush or as a flosser, may also be arranged on a rather long sheet-layout, respectively strip, and may be connected via a perforated region. Per each sheet-layout, 2 to 24, preferably 4 to 16, particularly preferentially 6 to 12 interdental brushes, respectively flossers, are provided. In a further implementation variant, the strip may also be rolled at length to form a ribbon or may be folded in a flat fashion, such that it is insertable into a secondary packaging having a slit which serves as a dispenser. Herein respectively one product may be pulled out, respectively wound off and separated off, through the slit. The remaining products thus remain protected in the dispenser.

Optionally it would be conceivable that a pedestal can be provided by unfolding, respectively slightly inclining, a subregion of the layers of the flat paper material. The layers may for this purpose in particular be at least partially separated at a perforation by an end customer. However, the connected layers may also be bent apart, respectively interlaced, via a slitting in the handle portion. Alternatively, the layers may region-wise be formed at least partially so as to be unconnected, such that an end customer may region-wise pull the layers apart, respectively interlace the layers. These solutions can be used to form a pedestal and/or for the afore-mentioned separation of the layers in order to separate the interdental brush from the paper material on a material side for disposal. This will allow separate disposal of the paper material in the paper collection and of the application unit (interdental brush) in the household waste. This implementation is possible for all body care products made of a flat paper material and mentioned in this document, in particular for flossers, interdental brushes or toothbrushes.

Optionally, protruding portions and/or attenuations of the layers, caused by material displacement of the paper material which occurs when connecting the layers by the twisted wire, can be avoided by an insertion of an intermediate layer of paper material, wherein the twisted wire lies in the intermediate layer in a correspondingly pre-punched recess. Herein the intermediate layer in particular does not cover the upper and lower layers completely.

Optionally, the twisted end of the brush can be deformed in order to additionally increase the pull-out force of the twisted wire out of the paper material. For this, the end of the twisted-in brush may in particular be provided with a deformation. This allows attaining an improvement of the hold in the paper material as well as a distribution of the force in the application. The deformation of the application unit is in particular realized transversally to the longitudinal direction, for example in an L-shape or S-shape, in a wave-shape or clothes-hanger-like, the deformation extending on two sides of the twisted-in brush. The deformation of the twisted wire increases the pull-out force out of the handle body. The deformation may be carried out before or after the anchoring of the twisted wire in the paper material. The paper material may for this purpose be embossed in that region or may have a corresponding recess. The pull-out force of the twisted wire out of the handle body made of paper material is between 1.5 kg and 6 kg, preferably in the range of 3 kg to 4.5 kg.

Optionally, in the production of an interdental brush with layers and with a mostly flat implementation of the body, a protruding structure in the shape of a protruding nose may be formed in the region directly behind the brush. With this nose, on the side of the nose that faces away from the brush, the skin of the cheeks, respectively the corners of the mouth, can be pulled back, such that the skin of the cheeks does not get in the way when the interdental brush is introduced into the interdental spaces. There is also no friction between the body of the interdental brush and the skin of the cheek when the interdental brush is moved in the interdental spaces. The skin is pulled back by the protruding nose and thus the elements will not rub on each other.

If the body care product is realized as an interdental cleaner, it would further be conceivable that the application unit is made of plastic in an injection-molding procedure or an extrusion procedure, wherein a structure and/or manufacturing otherwise corresponds to the structure and/or manufacturing of the body care product in an implementation as an interdental brush. Optionally the application unit of the interdental cleaner may be a defined interface structure comprising elements for an anchoring between the flat paper material layer(s). Moreover, the application unit of the interdental cleaner may optionally be laid between two layers of paper material or is mounted onto layers of paper material.

If the body care product is realized as an interdental cleaner, it would moreover be conceivable that the application unit is made of a paper material, in particular a rolled paper material, of an organic material or of wood or bamboo. In the other aspects, the above-described structure and/or production of the body care product corresponds to the implementation as an interdental brush or of the thread, respectively the ribbon. It would also be conceivable that the application unit of the body care product is mounted at the material base body. In this regard, for example, a flosser with a corresponding material base body would be conceivable, wherein the dental floss thread is sewed with at least one layer of the material base body. It is herein in particular conceivable that the dental floss thread is sewed onto the first layer, the dental floss thread being used as the sewing means. Alternatively or additionally, the dental floss thread may be sewed on while a further thread is used for fixing the dental floss thread. Preferably, in such a connection in particular 1 to 6 layers, preferably 1 to 3 layers are provided. The paper material of the layers is in particular provided in a thickness that is directly suitable for usage, such that no further connection processes are required to form the carrier. The layers in particular have a material thickness of 0.5 mm to 2 mm, preferably of 0.5 mm to 1.5 mm, with a density of 180 g/m² to 500 g/m², preferably 240 g/m² to 400 g/m², in particular so as to permit a connection by sewing. During production of a body care product, in particular if implemented as a flosser, it is thus in particular possible for the application unit to be sewed onto the material handle body. For this purpose, in a first method step in particular a sheet of paper material is at least partially punched as the first layer. In particular, at least such regions of the material handle body are punched in which the application unit is arranged. In particular, such regions are punched which cannot be punched any more after mounting the dental floss thread of the application unit. The punched sheet is then laid out and the dental floss thread is supplied and sewed on. The sewing may be realized either using the dental floss itself or with an additional thread. When the dental floss thread is used as a sewing means, the dental floss thread is in particular sewed tight at the two ends, in particular at the arms of the material handle body, the region of the dental floss thread spanned between the ends serving as the application region.

Optionally, in particular further layers of a paper material may be provided. For example, in particular a further sub-layer may be connected or folded onto a first sub-layer, thus creating several plies. For this the material handle body may, for example, have a hinge on top of the arms. This in particular allows avoiding injuries. It would also be conceivable that the sewing stitch is used as an optical design element. Alternatively or additionally, it would be conceivable that the lamination of the layers is additionally used as a closure. Alternatively or additionally, it would be conceivable that a fastening of the application unit is realized by an adhesive material or by molding-on in an injection molding. However, in particular tearing out the thread during the application must be prevented by means of the connection technique. In the case of sewing on, in particular only one punching step is necessary, wherein it is in particular difficult to hide the thread ends of the dental floss.

Furthermore, for this for example a flosser with a corresponding material base body would be conceivable as a body care product, wherein the dental floss thread is fixed in a recess by means of an adhesive material (e. g. hot-setting glue) or by molding on a plastic. It is herein in particular conceivable that the layers of the body are provided and have a recess. The recess may be a blind hole or a through hole. The dental floss thread is laid into the recess or is at least partially guided through the recess. Following this, the recess containing the thread is filled with hot-setting glue, plastic or something like that. The thread is thus fixed in the recess and is spanned between the two recesses. During production of a body care product, which is in particular implemented as a flosser, the application unit is thus in particular connectable to the material handle body in this manner. For this, in a first method step in particular the material base body is provided, in particular via a punching process. In particular, at least such regions of the material handle body are punched in which the application unit will be arranged. In particular, such regions are punched which cannot be punched any more after the mounting of the dental floss thread of the application unit. Then the punched sheet is laid out, and the dental floss thread is laid out and is laid out and laid into the recesses or is at least partially guided through the recesses. The fixing is then realized by hot-setting glue, by molding on, or something like that. In a utilization of the dental floss thread in a flosser, in particular respectively one recess is provided at the two ends, in particular at the arms of the material handle body, and is then handled accordingly, wherein the region of the dental floss thread spanned between the ends serves as the application region. Optionally, in particular further layers of paper material may be provided, which are applied onto the existing material handle body, for example so as to cover the connection structures and recesses. For example, in particular a further sub-layer may be connected or folded onto a first sub-layer so as to create several layers. In particular, the material handle body may for this purpose have a hinge on top of the arms. This in particular allows avoiding injuries.

In particular, an option is conceivable in which the number of layers of paper material varies. For example, in the region of the anchoring, where a defined stability must be achieved, there may be more layers than at the remaining handle—or vice versa. Not every layer must necessarily extend over the entire region of the adjoining layer. A reduced layer may be situated peripherally or may be arranged between larger layers. By an arrangement between the layers, damaging by tear-off can be avoided. There are in particular no exposed edges, which are bothersome and may constitute an injury risk.

As has been described, the layers may generally be formed from the same sheet, respectively sheet-layout, by folding and then sealing the layers. In this way hinge-like folding edges are created, for example—in the case of a flosser—on top of the arms or—in the case of an interdental brush—at the lower end (that is opposite the brush). The folded layer may be identical to or smaller than the non-folded layer of the sheet, respectively the sheet-layout.

Folding may be simplified by a fluting or a perforation, respectively the ply may be thus defined. However, preferably the outer side of the folding remains unaffected. A fluting or a perforation is preferably introduced on the inner side of the folding. Folded layers have the advantage that fewer cut edges of the paper material are exposed to humidity and water intake is thus reduced in that region.

A further possibility of protecting the cut edges of the paper material is beading. Individual or several layers of the cut edge may be at least partially beaded in a peripheral region. This provides the body care product with additional stability and reduces the injury risk, in particular with an application in the oral cavity.

It is also proposed that the material handle body of the handle unit comprises at least one layer of a paper material, which is realized so as to be at least partially folded. This in particular allows advantageous production of a handle body. It is in particular possible to provide a handle body that is advantageously stable and simply manufacturable. In particular, an advantageous handle shape can be provided. Preferably the layer is produced from a contiguous paper and/or cardboard, which is in particular cut to size. The layer advantageously has at least one folding axis and/or at least one folding edge. In a usage state, the layer is preferably folded at the at least one folding axis and/or the at least one folding edge. Advantageously, the at least one folding axis and/or folding edge facilitates folding of the layer into a usage state, or the product is given the form necessary for usage by folding. It would be conceivable that the layer is implemented so as to be perforated and/or fluted and/or embossed in the folding edge and/or folding axis in order to enable defined folding.

Preferably the layer is divided into two faces, in particular sub-layers, by the at least one folding axis. Preferably the at least one folding axis and/or folding edge runs parallel to the main extension direction of the body care product. It is also conceivable that at least one folding axis runs perpendicularly to the main extension direction of the body care product. The folding edge may be realized so as to be continuous or non-continuous; it is also possible that a combination of perforations, flutings and/or cuts forms the folding edge. The elements may, for example, be arranged one behind the other one. The folding edge may be implemented straight or bent. In this way it is also possible to design waisted shapes when the body has been assembled. The material handle body may herein in particular be folded from paper sheets or off a reel. Furthermore, a connection of folding faces may in particular be realized by sealing or gluing or mounting (like plugging, latching). For example, embossing is conceivable as a pre-treatment, aiming at creating a structure on the surface of the final product. Alternatively or additionally, coating is conceivable as a pre-treatment. For example, an inner face of a layer could be configured for gluing while an outer face has a protection against humidity. The layer may thus have two faces with different treatments, like for example coatings. Alternatively or additionally, printing is conceivable as a pre-treatment. It is also conceivable that the at least one layer is subjected to post-treatment after production. For example, coating, printing and/or laminating are/is conceivable as a post-treatment. Preferably the layer of the material handle body is cut or punched.

In particular, a toothbrush would be conceivable as a body care product with a corresponding material base body. The body care product is in particular produced in a flat fashion, wherein in particular flutings, embossings, perforations or the like are configured for a folding before a first use. The flutings are in particular arranged in the folding axis. Moreover, a combination of folding and subsequent connecting, like for example by pasting, sealing, welding, gluing, laminating, is conceivable. In the case of a body care product embodied as a toothbrush, the body care product could, origami-style, form a framework of paper. Alternatively or additionally, the body care product implemented as a toothbrush could comprise cleaning elements made of a paper material. The cleaning elements could be arranged on a bristle platelet made of plastic, of wood or of a paper material, wherein the bristle platelet is arranged, respectively fixed, on the paper material of the base body/handle body.

In particular, a toothpick or an interdental cleaner would be conceivable as a body care product with a corresponding material base body. The body care product is in particular made of a flat paper material, wherein in particular flutings are provided for folding before a first use. The flutings are in particular arranged in the folding axis. In use, the body care product may in particular be folded to form a triangle, or to form a pyramid-shaped or cone-shaped figure. The paper material preferentially used for the production of a body care product realized as a toothpick is a transparent paper. This transparent paper is preferably made of new cellulose (wood-free). For the application given above, the transparent paper has an area density of 150 g/m² to 300 g/m², preferably of 200 g/m² to 250 g/m². Optionally, in the case of a folded body care product a framework of paper material may be additionally provided within, the framework being enclosed by an envelope on the outside.

A toothbrush would be conceivable as a further body care product with a corresponding material base body (these options are also available for other body care products, like interdental brushes, flossers or safety razors). The paper material is prepared as a layer with a corresponding fluting, perforation or the like, such that then a respective product can be folded. The toothbrush produced in this manner from a paper material may be created with different cross section profiles. For example, the product may in the usage state form a closed profile, e. g. a triangular, tetragonal or generally polygonal profile, an open profile, e. g. a V-profile, a W-profile or a profile combined thereof. The profile eventually forms a body that implements the handle body of the toothbrush. Herein the profile may also serve, for example, to form a standing surface on the rear side of the toothbrush, such that the toothbrush can be deposited on a surface and does not roll around its own axis. The fluting, perforations or the like prepared for the foldings do not necessarily have to be taken over the entire body but may as well end in a surface, which may then for example result in a rounding. The assembly of the product may be realized in different ways. For example, the product may be already finally assembled or the end customer (user) assembles the product after the purchase, respectively before usage. The second variant may provide a variety of advantages with regard to the space required in the packaging, the body care product having less volume in the flat state than in the assembled state. In assembly different regions of the layer are connected to one another in order to form the voluminous body, respectively the handle portion. The layers may be connected directly by adhesive strips, by glue or by another agent suitable for the connection. The bristle field may be already mounted at the handle or may be fixed with mounting of the handle as well, for example by gluing. It may also be possible to detach certain regions of the paper material layer before assembly. It is further possible that there are elements designed for enabling a closed hollow space being formed by the body of the product in the state ready for use. Thus certain tabs are formed, which are plugged into openings and fixed therein, such that the openings are then closed. The foldings may be designed in different directions, preferably the largest portion of the foldings will take place around folding axes in the longitudinal direction. However, it is also possible that foldings are prepared in transversal directions, for example if elements are folded into each other for packaging. Furthermore, elements may be provided which are to be folded in such a way that, for example, the handle body is closed on the side of the application unit (e. g. the bristle field).

It is also possible to provide the paper material layers with structures and further surface coatings etc. in the flat state (before folding) in order to create surface elements in the final product which fulfil certain characteristics, for example have a higher friction. Beyond this it is proposed that the handle body comprises tension tabs, the application unit being anchored at the tension tabs of the material handle body. The tension tabs serve for an anchoring of the application unit, in particular in an implementation of the application unit as a flosser. It is moreover proposed that the cleaning element of the application unit is guided around the tension tabs. In particular, the thread or ribbon may in particular be guided around the tension tabs. This allows preventing tear-out later on when the tension tabs are fixed between the layers. The tension tabs have a length of 5 mm to 30 mm, preferably of 10 mm to 20 mm, with a width of 2 mm to 6 mm, preferably of 3 mm to 4 mm. The tension tabs are arranged so as to be situated next to the free space of the thread or ribbon. The thread or ribbon may furthermore be also fixed in a further point. For example, an adhesive dot may be configured for a fixing. Alternatively, the thread or ribbon may also be formed as a ring or noose, i. e. in particular as a closed ring having no actual end, such that a fixing in addition to the tension tabs may be dispensed with. The ring made of the thread or ribbon may herein be manufactured directly as a ring or may be produced by knotting. The paper material of the material handle body is in particular sealed during production, wherein the tension tabs are sealed in.

It is further proposed that the application unit is in at least one connection section glued and/or laminated in the material handle body in between the at least two layers. The application unit may be anchored in different manners which are deemed expedient by someone skilled in the art. An anchoring may be realized, for example, by a fastening of the application unit by means of an adhesive material. Alternatively or additionally, an anchoring may be realized by welding. The application unit may be fixed between the layers by welding.

It is also proposed that the application unit comprises at least one cleaning element carrier, in particular a wire, and several cleaning elements which are connected, in particular twisted-in, with the cleaning element carrier, wherein for a connection to the handle unit the cleaning element carrier is connected to the material handle body in a connection section facing away from the cleaning elements. This in particular allows providing an advantageous connection of the body care product. It is in particular possible to keep a number of elements of the body care product small. Preferably the cleaning elements are embodied as bristles. Preferentially the cleaning element carrier is coupled directly with the cleaning elements and is coupled directly with the material handle body. The cleaning element carrier is in particular realized integrally, preferably in a one-part implementation. “In a one-part implementation” is in particular to mean formed in one piece. Preferably this one piece is produced from a single blank, from a mass and/or a cast, particularly preferentially in an injection-molding procedure, in particular a one-component and/or multi-component injection-molding procedure. Particularly preferentially the cleaning element carrier is in a connection section, in particular directly, connected to a paper material of the material handle body.

Furthermore, it is proposed that the connection section of the cleaning element carrier of the application unit projects into the material handle body and is enclosed by the material handle body. It is in particular proposed that the at least one cleaning element carrier of the application unit is implemented so as to be rolled into at least one layer of the material handle body in a connection section facing away from the cleaning elements. This in particular allows providing an advantageous connection between the application unit and the handle unit. Alternatively, the cleaning element carrier of the application unit may be introduced into a hollow space of the material handle body in a connection section facing away from the cleaning elements. The hollow space may be provided during a production of the material handle body by rolling, embossing etc., or may be created with mechanical means, i. e. the hollow space is created by displacement (mandrel), drilling or something like that. Preferably the application unit is connected to the material handle body directly via the connection section. In one variant, the application unit may be connected to the material handle body while the material handle body is being rolled. The connection section of the application unit is herein in particular rolled into the material handle body. In a second variant, the application unit may be connected to the material handle body in the connection section after the material handle body has been rolled. The application unit may herein in particular be connected to the material handle body by plugging into and/or onto the material handle body. As has been mentioned, it is possible to mechanically process the material handle body beforehand in order to create the necessary free space for the connection section. It is furthermore possible, depending on the design of the connection section, to plug the connection section into the handle body without having prepared the handle body beforehand, for example by creating a recess. In such an implementation the connection section quasi at the same time forms the mandrel for creating the recess. In a third variant, the application unit may at least partially form part of the rolled portion of the handle body. The application unit may herein in particular be realized as an interdental brush, as a toothbrush head or as a toothpick.

The fixing of the connection section in the application unit is carried out using known methods like welding, sealing, pasting (gluing), pressing, laminating, etc.

In support of or instead of the fixing described, something like an embossing may be provided, the connection being realized not in a planar plane but by the surface being, for example, fluted or grooved in the connection section. In this way a more stable connection is achievable between the connection section and the handle body. Such elements may furthermore be introduced in other places as well so as to improve ergonomics and/or a hold.

The rolled or layered body may be embossed, respectively deformed, in such a way that an improved connection of the connection section, respectively of the fluted wire, results. The embossing is carried out by means of pressure and/or heat, as has been described. For this the paper layers may be slightly wetted, for example with water (vapor) so as to simplify re-forming. In this way, by a treatment with heat, humidity and pressure, the product may assume a shape that extends into a further dimension, for example a handle body having a bend or an angle.

Alternatively or additionally, the connection section of the application unit may be realized as an interface for an exchangeable part, like for example an exchangeable head for a manual toothbrush.

The material handle body may in particular be rolled in a straight manner or may be rolled in a spiral shape. It is also conceivable that the material handle body is rolled like a paper wiper, in particular a so-called estompe. The material handle body that is designed as a roll in particular has a (largest) diameter of 1 mm to 25 mm, preferably 1.5 mm to 20 mm, particularly preferentially 2 mm to 18 mm.

In particular, an interdental brush would be advantageous as a body care product with a corresponding material handle body. Herein the paper may in particular be rolled directly during a twisting-in of the brush. This in particular allows achieving that finished interdental brushes leave the machine directly. In one variant, a respective process flow for a production could herein be realized in such a way that in a first step the application unit is produced in an implementation as an interdental brush by twisting-in. After this, in a second step the at least one layer of the material handle body is rolled and at the same time a connection section of the application unit is rolled into the material handle body. An anchoring of the application unit in the material handle body may in particular be realized by an adhesive material, by welding, embossing, deformation or pressing, or by combinations thereof. For this possibly a further method step may be necessary. In a second variant for a production of the body care product, in a first step the application unit is produced as a twisted-in brush by twisting-in. In parallel thereto, the material handle body is produced by rolling the at least one layer. Then the material handle body is prepared for a connection to the application unit in a second step. For this purpose, for example, a hole is created in the material handle body. For this the handle body may be drilled on a front side or the material may be displaced using a mandrel. It would however also be conceivable that the hole is already created when producing the material handle body, for example by leaving an empty space (hollow cylinder), wherein in such a case in particular the second step (forming a hole mechanically) could be dispensed with.

Following this, the application unit is anchored in a recess in the material handle body in a further method step, for example by gluing in, welding, pressing, sealing or the like. It is furthermore possible, depending on a design of the connection section, to plug the connection section into the handle body without having prepared the handle body beforehand, for example by creating a recess. In such an implementation the connection section quasi forms at the same time the mandrel for creating the hollow space. In a third variant for a production of the body care product, in a first step the application unit is produced as a twisted-in brush by twisting-in. In parallel thereto, the material handle body is produced by a rolling of the at least one layer, wherein the material handle body is in particular produced as a hollow tube. Then the application unit is anchored in the recess in the material handle body in a further method step, for example by gluing in, welding, pressing or the like. The application unit may in particular be plugged and anchored directly in the central hole of the tube.

Optionally, the material handle body may be produced of a paper material that is already printed. In this way a colored final product could be created without post-processing.

Alternatively or additionally, it would be conceivable that the shaft ends of the material handle body are closed. By closing the open sides, it is in particular possible to produce a compact section of the material handle body, such that water cannot enter into the edges of the paper material. For this in particular a mounted cover portion, respectively a cover, could be provided at the top and/or at the bottom. The cover portion may be a component of the application unit, respectively of the connection section. Alternatively or additionally, it would also be conceivable that the ends of the material handle body are pressed and/or beaded and/or folded and/or are for example treated, respectively sealed, with water-repellent agents, like wax, varnish or the like. In the case of a sealing, it would for example be conceivable that a filling material, like for example wax, is already provided on the paper material and only the ends of the material handle body must be heated for a sealing. In particular, creating the stable interface between the paper material and the application unit could be considered as a difficulty.

In an implementation of the body care product as an interdental cleaner, it would in particular also be conceivable that the application unit is produced in an injection-molding procedure, for example in a fiber cast process/paper injection-molding process, wherein a structure and/or production otherwise corresponds to the structure and/or the production of the body care product as an interdental brush.

It is moreover proposed that the connection section of the cleaning element carrier has an angled structure for an anchoring in the handle body. It is also proposed that the at least one cleaning element carrier of the application unit is realized so as to be bent in the connection section for an at least partially form-fit connection to the handle unit. Preferably, for an at least partially form-fit connection to the application unit, the at least one cleaning element carrier of the application unit is realized so as to be bent in the connection section between two layers of the material handle body, which are in particular connected to each other in a layered manner. This in particular allows avoiding a tear-out of the cleaning element carrier. It is in particular possible to provide an advantageously stable body care product. In particular, a twisted-in brush would be conceivable as a body care product with a corresponding connection. In particular, the end, respectively the twisted-in wire, of the twisted-in brush may for this purpose be provided with a deformation. In this way an improved hold in the paper material as well as a force distribution in an application are achievable. The deformation of the application unit is in particular realized transversally to the longitudinal direction, for example in an L-shape, an S-shape, a wave shape or in a clothes-hanger-like manner, the deformation extending on two sides of the twisted-in brush. Alternatively or additionally, it would be conceivable that the connection section extends substantially through the entire material handle body, wherein the handle body may be bent into a desired shape by the connection section, in particular if implemented as a wire. It is herein possible to use the connection section as a forming element, respectively as an adjusting element, for example such that the handle unit may be given a non-planar, respectively three-dimensional, shape, for example a handle unit having a bulge, a twisting, an angle, etc. Alternatively or additionally, it would be conceivable that a wire loop is laid into the handle body via the connection section, by which a ring-shaped elevation is created on the handle body that may, for example, be used as a thumb grip.

Furthermore, it is proposed that the at least one cleaning element carrier of the application unit is connected to the material handle body at least partially by substance-to-substance bond, in particular by gluing, sealing, laminating and/or welding. This in particular allows avoiding a tear-out of the cleaning element carrier. It is in particular possible to provide an advantageously stable body care product.

It is further proposed that the application unit has at least one cleaning element which comprises a paper material. The cleaning element may herein in particular be realized as individual bristles or as a bristle bundle, also as a dental floss thread, as a dental floss ribbon, in particular an interdental tape, as a film or the like. Different implementations of the cleaning element are conceivable which are deemed expedient by someone skilled in the art. In particular, the handle unit and the application unit could be produced from the same paper material, respectively the same paper sheet. A massive handle unit and a sparsely-furnished bristle field of the application unit may be produced by folding, rolling and fixing. Alternatively, the application unit may be layered and pasted, the cleaning elements being punched from a sheet. The cleaning elements could in particular consist of a mechanically processed paper material; for example, the paper material may be slitted, cut, singulated, punched, pressed, compacted, etc. Possible slits in the paper material may be realized having a distance or having no distance. However, in particular a lateral distance is provided to allow sidewise movement of the cleaning elements. For this purpose, in particular different slitted layers and non-slitted layers, respectively reduced layers, are provided above one another. Beyond this, a hybrid solution would also be conceivable, for example of paper in a combination with plastic, in the manner already provided with yoghurt cups. In particular, a variant having paper within and plastic without would also be conceivable.

This in particular allows providing an advantageously ecological body care product. In particular, advantageously simple disposal of the body care product is achievable.

It is also proposed that the at least one cleaning element is at least partially implemented by a film or a ribbon, which may be realized as an interdental cleaner. The cleaning element is in particular embodied as a thread, as a ribbon, respectively as an interdental tape. Preferably the application unit, in particular the cleaning element, is implemented as an interdental cleaner having a thread-like or film-like structure, based for example on plastic and/or paper. Herein in particular a tearproof paper, a plastic film consisting of a hard or soft component, like for example a plastic monofilm or composite film, i. e. in particular several connected monofilms, a sustainable and/or degradable material, like for example PLA, and/or a non-woven-like material, like for example Tyvek from the DuPont company, may be used as a basic material. The interdental cleaner is herein processed in the same way as the dental floss described above. The interdental cleaner is in particular connected to the handle unit between layers of the material handle body. A product structure may herein in particular, at least section-wise, comprise at least three layers: a first layer consisting of a paper material, a second layer realized as the interdental cleaner, and a third layer consisting of a paper material. The body care product is in particular not three-layered in all places and/or does not have the same number of layers in all places. The interdental cleaner is in particular inserted only in a sub-region. Furthermore, the interdental cleaner may in particular be processed mechanically. The interdental cleaner may be provided with an embossment, in particular so as to create surface structures and a topography. The interdental cleaner may be folded on a usage side. The interdental cleaner may moreover be provided with additives, like for example antibacterial agents, cleaning agents, like in particular oral hygiene cleaning agents, flavor additives and/or abrasive agents, like for example a granulation to support cleaning. It is further possible to create a surface structure by means of the abrasive agent. Beyond this it is conceivable that the interdental cleaner has different edges with different shapes. The interdental cleaner in particular has a thickness of 0.05 mm to 0.5 mm, preferably of 0.15 mm to 0.35 mm. The interdental cleaner furthermore in particular has a width or height of 0.5 mm to 10 mm, preferably of 1 mm to 6 mm. Its length is adapted to the free space in the application unit.

It is also proposed that the application unit is arranged at the handle unit between the at least two layers of the material handle body. The application unit is in particular implemented by an interdental brush, a dental floss and/or an interdental cleaner. This in particular allows providing an advantageously compact and stable structure.

Furthermore, it is proposed that the application unit comprises at least one cleaning element implemented as a thread, which is in at least one connection section realized so as to be partially melted. The cleaning element is in particular partially melted at its ends for a connection to the material handle body. A thread is preferably made of a hard component (e. g. polyamide, polyester) or of a renewable plastic material or of Teflon. It is moreover proposed that the application unit comprises at least one cleaning element, which is in particular implemented as a thread, and which is connected to the material handle body by means of two parallel fixings. Preferably the cleaning element is connected to the material handle body in parallel by gluing, in particular by means of a hot-setting glue, and by partial melting. In particular, in parallel or additionally, a sideways fixing may be realized by a partial melting of the thread/ribbon. The thread ends or ribbon ends are herein melted by heat when exiting the sealed region. By the melting a mushroom-shaped/bulb-shaped end is formed increasing the pull-out force; this means that there will be less tear-out of the thread, respectively the ribbon, under load. The mushroom-shaped/bulb-shaped outer end of the thread, respectively the ribbon, has a larger diameter than the thread and abuts on the paper material on the outside. The force acting onto the thread, respectively the ribbon, is thus better transferred to the holding geometry and there will be less tear-out of the thread, respectively the ribbon. Partial melting does not work with any materials for threads, respectively ribbons; the material must be naturally meltable and must not just burn or resist the heat. Due to the dimensions, the thread, respectively the ribbon, optimally projects by 3 mm to 10 mm, preferably by 4 mm to 6 mm, before it is partially melted. This yields an optimum size of the mushroom-shaped/bulb-shaped end. The largest diameter is 0.2 mm to 2.5 mm, preferably 0.5 mm to 2 mm, particularly preferentially 0.7 mm to 1.5 mm. The partial melting is optimally not realized with a naked flame but with a hot stamp. The mushroom-shaped/bulb-shaped shape may be formed by the hot stamp via pressing on.

It is in particular also conceivable that different anchoring techniques are combined, like for example welding and surface structures, slitting and welding, tension tabs and welding, tension tabs and gluing as well as tension tabs, surface structures and welding. It is furthermore possible to combine the connection techniques mentioned with a partial melting of the thread ends/ribbon ends. Herein in particular different connection techniques and/or combinations of connection techniques are conceivable which are deemed expedient by someone skilled in the art. Principally, any of the connection techniques mentioned may be combined with one another. It is herein possible to use combinations of two, three or four techniques. However, in particular a tear-out of the thread or of the ribbon in an application must be prevented by the anchoring technique. Preferably a corresponding construction in particular allows achieving that the flosser is disposable directly with the paper collection or cardboard collection.

It is also proposed that the body care product comprises a protective element, which is connected with the handle unit and is releasably connected with the handle unit. The protective element may in particular be produced integrally with the handle unit, in particular the material handle body, wherein for example a perforation permits a separation of the components. The protective element is in particular realized by a covering cap. In the field of body care products, protective elements are often used for the application units, for example as head containers for manual toothbrushes or for plug-on brushes of electrical toothbrushes, for flossers, for interdental cleaners or for interdental brushes. It is now proposed to produce such protective elements of a paper material as well. The protective elements may be manufactured separately and then mounted on the body care product, or they may form a more integral component of the body care product which is removed, respectively pivoted away, respectively folded away, for usage.

The head container for a protection of the application unit may be realized, for example, on the basis of the shape of a hollow roll, respectively cylinder, which can be put over the application unit, for example an interdental brush or a toothbrush. Herein the protective element, respectively the head container, is mostly slid over the application unit in a direction of the longitudinal axis of the product. The hollow roll may herein be equipped with a further element for optimizing the functionality. For example, the hollow roll may be closed, respectively sealed at one end such that it cannot be slid too far over the application unit (the closed side also forming an abutment and being mounted at the upper end of the body care product), and that the application unit has enhanced protection. Furthermore, elements may be inserted in the hollow space of the roll, which facilitate an insertion of the application unit or for a fixing or an orientation of the application unit in the inserted state. It is also possible to accommodate the entire product in a roll of paper material, thus forming a protection for the entire product. The further functional elements may also be inserted here. Beyond this, it is in this case also possible to design the hollow roll as a packaging, such that no further packaging needs to be created.

A further implementation variant for protective elements is the integral forming of the elements during production. The body of the body care product, which is structured in layers, comprises designated separation means, for example perforations or separable material attenuations, which form a connection of the body with the protective element. For example, an interdental brush may have a handle body consisting of paper material layers. The paper material layers are designed such that they form the handle body on the one hand, on the other hand forming a protective element. The transition from the one part to the other part is provided with a perforation or a material attenuation that allows removing the protective element, in which case the application unit is exposed. In this way it is for example possible to create an interdental brush whose application unit is fixed between layers of paper material via the connection section. The layers extend from the handle body via a perforation to the protective element. The protective element lies embedded between layers of paper material, wherein the layers may, depending on their embossments, form a hollow space between them in which the cleaning elements are situated. Separating the layer above the protective element, respectively breaking the perforation, will allow the layers above the protective element being pulled off and the application unit will be exposed. Furthermore, a flosser may also be provided with a protective element. The protective element is situated over the dental floss and can be pulled off, whereby the empty space between the dental floss and the handle body will be exposed.

In this implementation the protective element may, in addition to the protective function, also serve as a first-opening guarantee, respectively first-opening indication. The closed product indicates that it has not been used. If it is open, first use is no longer guaranteed.

It is further proposed that the body care product comprises a plug connection unit configured for a releasable plug connection of the application unit, which is in particular embodied as a toothbrush head, to the handle unit. Herein it would in particular also be conceivable that the application unit is embodied as an exchangeable head, the plug connection unit being arranged directly between the application unit and the handle unit. The plug connection unit is in particular realized at least partially integrally with the application unit and/or at least partially integrally with the handle unit. Preferably the plug connection unit comprises at least one first plug connection element, which is fixedly connected with the handle unit, and at least one second plug connection element corresponding to the first plug connection element, which is fixedly connected with the application unit. In particular, the application unit is separable from the material handle body via the plug connection unit. The plug connection unit is in particular configured to provide a releasable or non-releasable latch connection. The first plug connection element in particular forms a latch projection while the second plug connection element forms a latch recess corresponding to the first plug connection element. However, a vice versa implementation and/or a different implementation of the plug connection unit, deemed expedient by someone skilled in the art, would also be conceivable.

It is moreover proposed that the material handle body of the handle unit comprises several sub-elements which are configured to be plugged together. It would in particular be conceivable that the material handle body of the handle unit is at least partially plugged together from several sub-elements which are made of a paper material. For this, in particular a paper material is cut into the sub-elements and then the sub-elements are plugged together. In a basic method, a paper material is in particular cut into different shapes, then plugged together and optionally provided with a securing/connection. The connection may be realized, for example, by plugging, gluing and/or welding. For example, embossing is conceivable as a pre-treatment, aiming at creating a structure on the surface of the final product. Alternatively or additionally, coating is conceivable as a pre-treatment, wherein an outer face has a protection against humidity. Alternatively or additionally, printing is conceivable as a pre-treatment. It is further conceivable that the at least one layer is subjected to post-treatment after production. For example, coating, printing and/or laminating is conceivable as a post-treatment. The post-treatment may be carried out after a production of the sub-elements or after assembly of the sub-elements. It is in particular possible to provide, in particular afore-described, in particular flat, handle bodies, like in particular the afore-described flossers, with at least one additional sub-element which essentially gets rotated outwards in a further direction, which in particular runs perpendicularly to a main extension plane, thus in particular creating a 3D effect. Alternatively, the body care product may be embodied as a toothbrush, wherein for example a plastic part—as a base body—is equipped with geometries permitting a combination with pieces of paper material. This in particular allows providing an advantageously three-dimensional handle unit while keeping a material input at a low level. Preferably, in the case of a toothbrush, the application unit may be formed having a brush head and a neck prolongation of plastic, to which the material handle body with the sub-elements is plugged. The body care product may herein in particular consist of 2 to 25, preferably of 4 to 12, individual parts.

It is also proposed that the at least one application unit comprises a head region that forms a brush head. It is in particular proposed that in an implementation of the body care product as a toothbrush, the at least one application unit comprises a brush head, in particular a toothbrush head. The application unit advantageously comprises at least one cleaning unit, in particular a toothbrush head, with bristles. Advantageously, the cleaning unit moreover comprises at least one bristle carrier, for example a brush head base body. It is moreover proposed that a base body of the head region is completely made of a hard component and forms a bristle carrier. Alternatively or additionally, it is proposed that the body care product comprises a bristle platelet, which is accommodated in the head region and has a plurality of bristle bundles. For this purpose, first of all bristle platelets with through holes are manufactured by injection-molding, and then bristles are guided through the through holes. Preferably the bristles are then at least bundle-wise connected, in particular melted, preferably with one another and/or with the respective bristle platelet, on a rear side. Bristle platelets furnished with bristles in this manner may then be connected, for example welded, sealed and/or glued, connected mechanically by riveting, caulking, preferably by ultrasound welding, to a base body, in particular a brush head. For this the base body, in particular the brush head, preferably has a recess in which the bristle platelet is insertable.

In an implementation of the body care product as a flosser, it is in particular conceivable that different flat pieces of paper material, in particular for the handle unit, are cut and are then plugged together to form a 3D contour, respectively a 3D body. In a production in particular the application unit and the sub-elements of the handle unit are manufactured separately. Plugging together of the body care product may herein be realized already during production but also by a user himself. In the case of a plugging together carried out by a user, it is in particular possible to keep a packaging size of the body care product advantageously small. Herein in particular different construction variants are conceivable. In particular, a gridwork of sub-elements may be formed without a core, or a core is provided which the sub-elements are plugged onto. Herein in particular a stability of the connections must be provided which is required with regard to the application. In particular, falling apart must be avoided, for example using undercuts.

In an implementation of the body care product as an interdental cleaner, it would in particular be conceivable that the application unit is manufactured in an injection-molding process, wherein the application unit is plugged onto a plugged-together handle unit made of a paper material.

Moreover, it is proposed that the bristle platelet is produced from a paper material. The bristle platelet may be punched from a paper material. For this one or several layer/s of paper material may be provided, which form the body of the bristle platelet. The recesses are formed subsequently, for this a punching process, laser cutting, milling/drilling or other methods suitable for the processing of a paper material may be used. The bristles are in their turn connected to the bristle platelet in an AFT procedure, wherein the bristle platelet can be fixed in the handle unit in the corresponding recess or on the handle unit subsequently.

Beyond this it is proposed that the material handle body of the handle unit comprises at least one layer of paper material which is realized so as to be at least partially folded, the handle body having a stowage position, in which an inner face of the layer is oriented outwards, and an operational position, in which an outer face of the layer is oriented outwards. The handle unit may thus be in particular be folded inwards when packaged and may be folded outwards for usage. It is herein in particular conceivable that the handle body is combined with a packaging. Preferably, in a presentation state, the layer forms at least a portion of a packaging of the body care product. Preferably, transfer of the handle body from the stowage position into an operational position is irreversible as possibly certain elements must be separated off in order to get from the stowage position to the operational position.

Furthermore, it is proposed that the head region comprises an injection-molded bristle field. In particular, bristles are conceivable which are injection-molded on directly. These may in particular be manufactured together with the application unit, the handle unit and/or the coupling unit during a multi-component injection molding, or may be molded onto a base body of the application unit subsequently. Differently than with conventional bristles, injection-molded bristles are not extruded but are formed via an injection-molding procedure. Special materials are used for this. Injection-molded bristles may be injection-molded to the paper material or to wood directly, which means that the materials are laid into the injection-molding tool and are then overmolded. Alternatively, a bristle platelet with injection-molded bristles is created in the manner described above and is then connected to the paper material. For this, the aforementioned connection techniques are possible. As the bristle platelets are small and there is a risk of them being swallowed, they are preferably connected to the base body non-releasably. Detaching the injection-molded bristles, respectively the bristle platelet, is intended to occur only once (for example for separate disposal). A predetermined breaking point may be provided for this purpose.

It is also proposed that the material handle body of the handle unit comprises at least one layer of paper material, which is realized so as to be at least partially folded and to be connected by at least one plug connection, wherein at least one first folded plug connection forms a thumb grip of the material handle body. Preferably the at least one layer, in particular several layers, is/are connected via form-fit and/or force-fit plug connections. In particular, tabs may be provided which are plugged into recesses such that a connection is created. Specially arranged tabs may be designed so as to automatically form further elements, for example a thumb grip. This in particular allows creating an advantageous three-dimensional shape of the handle unit. It is furthermore possible that the handle unit is made ready for use by an operator himself. This allows keeping a transport volume of the body care product small.

Furthermore, it is proposed that the body care product is at least partially milled from a paper material. The body care product is herein in particular produced at least partially from solid fiberboard milled into a defined shape. Herein it is in particular conceivable that the at least one solid fiberboard is subjected to post-treatment after a milling. The solid fiberboard may also be made of several layers of a paper material. For example, coating, printing and/or laminating are/is conceivable as a post-treatment. Preferably, in particular at least the handle unit is made of solid fiberboard. Different solid fiberboards, deemed expedient by someone skilled in the art, are conceivable, like in particular finnboard. In this context, in particular a punched toothbrush would be conceivable as a body care product. For this, in particular a block of paper material is milled to form a contour, wherein holes are drilled into the future brush head. In production, in particular in a first step the handle unit and parts of the application unit are milled and drilled from a block of paper material. After this the handle unit and/or the application unit may optionally be printed and varnished. Following this bristle bundles of the application unit are punched, in particular inserted and profiled. Herein it is in particular necessary to provide a defined pull-out weight of the bristle bundles. Alternatively, it would be conceivable that the bristle bundles are fastened via AFT. For this the handle unit and parts of the application unit are milled from a block of paper material. A recess is created in the future brush head of the application unit. Parallel thereto a bristle platelet is manufactured, which can then be anchored in the recess. In production, for this purpose, in a first step the handle unit and parts of the application unit are in particular milled from a block of paper material, wherein a recess is introduced directly in a brush head of the application unit. Then the handle unit and/or the application unit may optionally be printed and varnished. Following this, a bristle platelet is injection-molded and is furnished with bristles. After this the finished bristle platelet may be inserted in the brush head of the application unit, and may be fixed by caulking and/or gluing. Herein the bristle platelet must in particular be reliably connected to the paper material.

It is moreover proposed that the body care product is at least partially cast from a paper material. The paper material is herein in particular processed in a procedure similar to injection molding. However, the processing of the paper material is implemented at considerably lower temperatures and injection pressures. Corresponding methods are known, for example, in the case of PurePaperPak, which is a custom protective paper packaging. Herein in particular ecological raw-material input is possible, the contained materials being of natural origin and in particular consisting by 100% of natural resources. The raw products in particular consist of industrial starch, preferably from potatoes, paper fibers, water, and an additive mix. The paper mixture created in this way is then used for further processing by an injection-molding technique. The paper injection molding is in particular realized in three steps. In a first step an injection is carried out, wherein the paper mixture is injected into an aluminum tool. In a second step a baking process is realized, in which the paper mixture is baked, in particular heated, in the tool. In a third step unforming is carried out, in which the finished packaging may be taken out. Furthermore, so-called PAPACKS® packagings are known as packagings. The packagings are in particular made of a PAPACKS® fiber cast certified for use with food products. Such products are in particular compostable and may also be entered into the waste paper cycle or bio-waste cycle. Herein it is in particular conceivable that the cast body care product is post-treated after casting. For example, coating, printing and/or laminating are/is conceivable as a post-treatment. In this context, in particular a punched toothbrush is conceivable as a body care product. For this a toothbrush body, in particular the handle unit and parts of the application unit, is/are in particular cast from a paper material and bristle holes are formed, preferably analogously or similar to actual injection molding. In production, in particular in a first step the handle unit and parts of the application unit are cast from a paper material. Then the handle unit and/or the application unit may optionally be printed and varnished. Following this, bristles of the application unit are punched, in particular inserted and profiled.

For an injection molding with a paper material, in particular paper with starch (industrial starch) and a solving agent may be used as materials. The components are mixed and are processed by suitable injection-molding machines. The materials are mixed with a ratio of 25% to 70%, preferably 30% to 40% paper, 20% to 40%, preferably 25% to 35% starch and 5% to 40%, preferably 10% to 35% solving agent. An industrial starch made, for example, of corn or potatoes may be used as a starch. The solving agent is in particular polyvinyl alcohol. For the mixing, the components are dissolved in water and mixed, and the resulting liquid/mass is dried and then processed to form a granulate.

It is further proposed that the paper material of the body care product, in particular of the handle unit, is at least section-wise implemented so as to be overmolded. The paper material may herein be provided in particular according to one of the aforementioned implementations. The paper material of the body care product, in particular of the handle unit, is in particular overmolded with a plastic. A connection of the overmolding to the paper material may herein in particular be achieved by undercuts, breakthroughs, i. e. in particular a force-fit and/or form-fit connection, and/or chemically, like in particular by a material bond. The material bond may also be realized by the injection-molded component entering the structure of the paper material. Due to the injection molding, the paper material is compressed in the overmolded portion. In order to seal the paper material with respect to the injected plastic in the injection-molding tool, the paper material is also compressed locally in the injection-molding tool. For this the paper material may in particular be implemented so as to be pre-treated, for example by special varnishes and/or coatings, in particular before the overmolding, so as to enable the connection. For example, a cleaning unit of the application unit may be created at least partially by the overmolding. It is in particular possible to create by overmolding a perforation field or a recess for a bristle platelet in a brush head of the application unit. Alternatively or additionally, injection-molded bristles may be provided which are produced directly by means of the overmolding. In this context, in particular a toothbrush would be conceivable as a body care product. For this purpose, in particular a cardboard tube or one or several layers of paper material is/are overmolded, wherein in particular a brush head is injection-molded directly onto the cardboard tube or the layer/s. In a production, in particular in a first step a cardboard tube, respectively the layer/s of paper material, is/are produced, which form the handle unit. Then the cardboard tube, respectively the layer/s, of paper material is/are overmolded, wherein in particular the application unit is formed with a field of perforations. Following this, bristles are punched, in particular inserted and profiled, into the field of perforations. Alternatively, in particular a flosser would be conceivable in this context as a body care product. For this in particular a cardboard tube, respectively the layer/s of paper material, is/are overmolded, wherein in particular a flosser head is injection-molded directly onto the cardboard tube, respectively the layer/s of paper material. In a production, in particular in a first step a cardboard tube, respectively the layer/s of paper material, is/are produced, which form the handle unit. Then the cardboard tube, respectively the layer/s of paper material, is/are overmolded, wherein in particular the application unit is formed as a flosser head. Alternatively, in this context in particular an interdental cleaner would be conceivable as a body care product. For this in particular a cardboard tube, respectively the layer/s of paper material, is/are overmolded, wherein in particular an interdental cleaner is injection-molded directly onto the cardboard tube, respectively the layer/s of paper material. In a production, in particular in a first step a cardboard tube is produced which forms the handle unit. Then the cardboard tube, respectively the layer/s of paper material, is/are overmolded, wherein in particular the application unit is formed as an interdental cleaner. Furthermore, a handle unit may be provided with an interface for exchanging the body care product. In a production, in particular in a first step a cardboard tube, respectively the layer/s of paper material, is/are produced which form the handle unit. Following this, the cardboard tube is overmolded, wherein in particular an interface with an application unit is formed. The application unit is equipped with the counter-piece to the interface and may thus comprise a great variety of applications, for example flossers, toothbrushes, interdental cleaners. It is also proposed that the body care product comprises at least one functional component, which is overmolded with a paper material. The functional component may herein in particular be implemented by a core of the handle unit and/or of the application unit, which is produced in a known method, for example a plastic injection-molding procedure. A connection of the overmolding to the functional component may herein in particular be achieved by undercuts, breakthroughs, i. e. in particular a force-fit and/or form-fit connection, or chemically, like in particular by a material bond. For this the functional component may in particular be implemented so as to be pre-treated, in particular before the overmolding, for creating the connection, for example by means of special varnishes and/or coatings. In this context, in particular an interdental cleaner or a toothbrush would be conceivable as a body care product. For this purpose, in particular the application unit and/or parts of the handle unit is/are produced in a plastic injection-molding procedure, wherein the application unit and/or parts of the handle unit are partially overmolded with a paper material.

The paper material of the body care product, in particular of the handle unit, may also be injection-molded separately with an injection-molded part with at least one functional component, such that it is in a subsequent process connected to the paper material, which is for example realized as a cardboard tube, respectively to the layer/s of paper material. This is possible for aforementioned examples (e. g. a brush head with injection-molded bristles or bristle platelets with conventional/extruded filaments, as a toothbrush, interdental cleaner, flosser, etc.). Herein elements for a connection to the paper material are provided at the at least one injection-molded part with at least one functional component. The connection is then realized by gluing, pasting, sealing, riveting, deformation, etc.

If one or several layer/s of paper material are used, these may of course be processed before or after injection-molding, respectively connecting, — as has been described in other places of this document—using a variety of methods mentioned in this document (folded, deformed, embossed, fluted, upgraded, coated, etc.).

Beyond this, it is proposed that the body care product, in particular the material handle body, comprises at least one layer that is at least partially deformed and/or pressed and/or deep-drawn. The at least one layer may in particular be produced from one or several sheet/s, respectively sheet layout/s, of a paper material and/or may be rolled off reels. The body care product in particular comprises several layers. The layers, in particular of the material handle body, may be connected to one another, for example, by gluing, welding and/or laminating. It is further conceivable that the at least one layer is pre-treated before processing. For example, embossing is conceivable as a pre-treatment, aiming at creating a structure on the surface of the final product. Alternatively or additionally, coating is conceivable as a pre-treatment. For example, an inner face of each layer could be configured for gluing while an outer face has a protection against humidity. Each layer may thus have two faces with different coatings. Alternatively or additionally, printing is conceivable as a pre-treatment. It is also conceivable that the at least one layer is subjected to post-treatment after production. For example, coating, printing and/or laminating is conceivable as a post-treatment. In this context, in particular a toothpick or a flosser is conceivable as a body care product. However, an alternative general basic shape is correspondingly conceivable for a handle unit. The at least one layer of the body care product is in particular deformed, preferentially deep-drawn, in particular using pressure and heat. In this way, in particular for example a gripping geometry is created for improved ergonomics. It is in particular possible to provide a body care product with a voluminous handle unit. It would also be conceivable that the handle unit consists of two composed deep-drawn half-shells, which include a hollow space in a state when connected to each other. The connection of the half-shells may be realized, for example, by gluing, welding, sealing and/or plugging. In particular, for example the handle unit of a toothbrush may be produced in this manner.

Optionally, a grouping of the products may in particular be carried out already during production of the body care products. For example, in a manner that is similar to a current production of interdental cleaners, a group of flossers may be provided which are separated for usage. The body care products may in particular be already connected via the paper material, there being in particular a perforation by which the body care products are separable. This in particular allows attaining simplified processing, a production being carried out in groups and a separation being realized only for usage.

For creating a cylindrical structure, in particular an extrusion of paper material would be conceivable as an alternative to a rolling of paper material. As an alternative to a milling of paper material, in particular rotating a paper material would also be conceivable in order to create rotationally symmetrical geometries.

It would moreover be conceivable that functional cut edges and/or margins are provided at the body care product. In particular, paper having different edges may be provided, wherein the edges may have functionally differing properties. For example, a functional face may be realized in a wave shape while another face having no function is realized in a smooth fashion. However, a vice versa implementation would also be conceivable. In particular, subsequent treatment of the cut edges may be carried out in order to avoid, for example, injury risk or water intake. In particular, a mechanical post-treatment against sharp edges may be provided, like in particular breaking, beading, bending and/or embossing of the edges. Beyond this, coatings of wax or varnish may be provided.

Furthermore, a special surface of the paper material may be created. In particular, on different faces different surfaces may be created, for example with regard to color or surface characteristics.

It is also conceivable that for the base, for example base feet, folding and/or interlacing of elements is provided. In particular, the paper material may be folded at one product end, such that the body care product may be stored standing up. It is in particular possible to create a pedestal.

It is also possible to induce flexibility by section-wise reduction of material thickness. In particular, bending edges may be created. A corresponding reduction of material thickness may be induced, for example, by fluting in the case of flat variants or by perforation.

According to the disclosure of the invention, a connection by welding is not necessarily realized over the entire contacting surface. For example, it is possible that tear-open tabs and/or certain peripheral positions, respectively peripheral zones, are not welded. Furthermore, a welding may for example be executed only at an edge while inner areas are left out. It would moreover also be conceivable that weldings are left out, for example so as to create mobility. Alternatively, it would also be conceivable that welding is intentionally done in order to provide mobility, in particular in a region of a folding edge. Alternatively or additionally, it is possible to weld only in the region of the interface with the application unit, for example only at the connection section of the cleaning element carrier.

If the body care product is implemented as an interdental brush, the handle unit is in particular more lightweight than a known plastic handle. An interdental brush with a rolled or layered handle body has a total weight of less than 2 g, preferably of less than 1 g, most preferentially of less than 0.6 g. A flosser having a layered handle body has a total weight of less than 2 g, preferably of less than 1.2 g, most preferentially of less than 0.8 g. The center of gravity of the interdental brush having a rolled or layered handle body is, measured from the lower end, at 40% to 70%, preferably at 45% to 60%, of a total length.

It is further proposed that the paper material has a water-repellent coating and/or impregnation. A suitable coating and/or impregnation allows achieving reduced water intake of the paper material. The paper material may thus be suitable for multiple use. An impregnation may herein be realized, for example, by a plastic and/or a wax or the like.

Beyond this it is proposed that the paper material is realized so as to be varnished and/or printed. This in particular allows attaining an advantageous individualization of the paper material. In particular, advantageously simple printing is enabled on a paper material. In particular, upgrading is achievable. An upgrading may, for example, be a varnishing, wherein for example a UV high-gloss varnish, a structure lacquer, a soft-touch varnish or a glitter varnish may be used. Alternatively or additionally, the paper material may have a printing. For example, the paper material may have a printing of water-based varnishes, which are ecological with respect to conventional varnishes.

The invention also relates to a system with several, in particular identically implemented, body care products, which in each case have a material handle body, the material handle bodies of the body care products being connected to each other in such a way that they are separable at perforations.

The invention is also based on a sales unit with at least one packaging, which is at least partially made of a paper material, and with at least one body care product, wherein a material handle body of the handle unit of the body care product comprises at least one layer of a paper material, wherein the layer is implemented at least partially integrally with the packaging. Preferably the body care product is realized as a foldable toothbrush. Preferably the packaging forms part of the future product. For example, a product with a mounted bristle field is provided in a packaging. The layer of paper material is folded in such a way that the bristle field and the portions contactable when in use are located within the packaging. For this the product is folded inversely with respect to a usage state, the outer surfaces of the product ready for use constituting in the packaged state the inner surfaces of the product. The product is quasi turned inside out. Herein it may also be possible that certain zones of the layer of paper material are separated off before mounting to form the ready-for-use state.

Furthermore, the invention is based on a method for producing a body care product. It is proposed that the material handle body of the handle unit is rolled onto at least one layer of paper material. This in particular allows producing an advantageously stable handle body. Preferably the layer of paper material is rolled up around a defined rolling axis. The rolling axis preferably runs parallel to, in particular coaxially with, a longitudinal axis of the handle unit. Preferentially, the layer is at least section-wise, in particular completely, rolled in a plane perpendicular to a longitudinal axis of the handle unit in order to form a spiral-shaped cross section. A largest surface of the layer in particular extends around a longitudinal axis of the handle unit. Preferably the at least one layer of the material handle body is rolled to form a cylindrical shape.

It is further proposed that the application unit is in at least one connection section rolled into the material handle body. This in particular allows providing an advantageous connection between the application unit and the handle unit.

It is moreover proposed that the material handle body of the handle unit is layered from at least two interconnected layers of a paper material. In this way, in particular an advantageous production of a handle body is possible. In particular, a handle body is provided which is advantageously stable and easily manufacturable. In a production method, in particular sheets of paper material, which form the layers, are pre-punched. A pre-punching is in particular executed in the region of the arrangement of the functional element, in particular the application unit. In particular, pre-punching is carried out of such regions of the material handle body which can no longer be punched after a mounting of the functional element. Then in particular the pre-punched sheet is laid out and a thread is laid therein. Following this, a further pre-punched sheet is laid on and the sheets are connected, like for example by laminating, welding and/or gluing. After this the body care product, in particular its outer geometry, can be subjected to final punching and surplus thread can be removed. It would also be conceivable that such a step is dispensed with and instead of pre-punching, final punching is directly carried out, wherein the thread directly does not protrude beyond the final outer geometry.

Beyond this it is proposed that the application unit is in at least one connection section glued and/or laminated into the material handle body in between the at least two layers. The application unit may be anchored in different ways deemed expedient by someone skilled in the art. An anchoring may be realized, for example, by fixing the application unit by means of an adhesive material. Alternatively or additionally, anchoring may be realized by welding. The application unit may be fastened between the layers by welding.

The aforementioned implementations of body care products also permit combinations.

### EXAMPLES

An interdental brush with a rolled handle body may be pointed at an end opposite the brush in such a way that a toothpick is formed.
A layered body that is implemented as a flosser may comprise a toothpick or an interdental brush at an end opposite the flosser.
A toothpick having a hollow handle body may accommodate an interdental brush with a rolled handle body in its hollow portion.

The body care product according to the invention shall herein not be limited to the applications and implementations described above. In particular, in order to fulfill a functionality that is described here, the body care product may comprise a number of individual elements, components and units that differs from a number given here, and/or may comprise any expedient combination of individual elements, components and units. Moreover, with regard to the value ranges given in the present disclosure, values situated within the limits mentioned shall also be considered as disclosed and as applicable according to requirements.

Of course the implementation variants illustrated in this document are exemplary. Within the scope of the invention, respective embodiments and elements of these implementation variants may be combined with other implementation variants without leaving the scope of the invention.

### DRAWINGS

Further advantages will become apparent from the following description of the drawings. In the drawings twenty-seven exemplary embodiments of the invention are illustrated. The drawings, the description and the claims contain a plurality of features in combination. Someone skilled in the art will purposefully also consider the features separately and will find further expedient combinations.

It is shown in:

FIG. 1A an application unit of a body care product according to the invention which is realized as an interdental brush, in a schematic illustration,

FIG. 1B the body care product according to the invention with the application unit and with a handle unit during production, in a schematic illustration,

FIG. 1C the finished body care product according to the invention with the application unit and with a handle unit, in a schematic illustration,

FIG. 1D a partial section of a layer of a material handle body of the handle unit, in a schematic illustration,

FIG. 2A a first layer of several handle units of respectively one alternative body care product according to the invention which is realized as a flosser, in a schematic illustration,

FIG. 2B the first layer of the several handle units, and several application units with depicted cleaning element of the alternative body care products according to the invention, in a schematic illustration,

FIG. 2C the handle units and the application units of the alternative body care products according to the invention during production, in a schematic illustration,

FIG. 2D the finished alternative body care product according to the invention with the application unit and with a handle unit, in a schematic illustration,

FIG. 3A a further alternative body care product according to the invention which is realized as a flosser, with an application unit and with a handle unit in a non-folded state during production, in a schematic illustration,

FIG. 3B the further alternative body care product according to the invention with the application unit and with the handle unit in a partially folded state during production, in a schematic illustration,

FIG. 3C the finished further alternative body care product according to the invention with the application unit and with a handle unit, in a schematic illustration,

FIG. 4A a first layer of several handle units of respectively one further alternative body care product according to the invention, realized as a flosser, during production, in a schematic illustration,

FIG. 4B the first layer of the several handle units, and several application units with depicted cleaning element, of the further alternative body care products according to the invention during production, in a schematic illustration,

FIG. 4C the handle units and the application units of the further alternative body care products according to the invention during production, in a schematic illustration,

FIG. 4D the finished further alternative body care product according to the invention with the application unit and with a handle unit, in a schematic illustration,

FIG. 5A an application unit of a further alternative body care product according to the invention, realized as an interdental brush, in a schematic illustration,

FIG. 5B a first layer of the several handle units, and several of the application units, of the further alternative body care products according to the invention during production, in a schematic illustration,

FIG. 5C the handle units and the application units of the further alternative body care products according to the invention during production, in a schematic illustration,

FIG. 5D the finished further alternative body care product according to the invention with the application unit and with a handle unit, in a schematic illustration,

FIG. 5E several finished further alternative body care products according to the invention, which are connected to one another, with the application unit and with a handle unit, in a schematic illustration,

FIG. 6A a handle unit of a further alternative body care product according to the invention which is realized as a flosser, in a schematic illustration,

FIG. 6B the finished further alternative body care product according to the invention with the application unit and with a handle unit, in a schematic illustration,

FIG. 7A a finished further alternative body care product according to the invention which is realized as a toothbrush, with an application unit and with a plugged handle unit, in a schematic illustration,

FIG. 7B the further alternative body care product according to the invention with the application unit and with the plugged handle unit, in a schematic exploded view,

FIG. 8A a further alternative body care product according to the invention which is realized as a toothpick, during production, in a schematic illustration,

FIG. 8B the finished further alternative body care product according to the invention, in a schematic 3D view,

FIG. 8C the finished further alternative body care product according to the invention, in a schematic side view,

FIG. 9A an application unit of a further alternative body care product according to the invention during production, in a schematic illustration,

FIG. 9B the finished further alternative body care product according to the invention with the application unit and with the handle unit, in a schematic illustration,

FIG. 10A a further alternative body care product according to the invention which is realized as a toothpick, in a non-folded state, in a schematic illustration,

FIG. 10B the further alternative body care product according to the invention in a folded application state, in a schematic illustration,

FIG. 11A a handle unit of a further alternative body care product according to the invention which is realized as a toothbrush, during production, in a schematic illustration,

FIG. 11B the finished further alternative body care product according to the invention with an application unit and with the handle unit, in a schematic illustration,

FIG. 12A an application unit of a further alternative body care product according to the invention which is realized as a toothbrush, during production, in a schematic illustration,

FIG. 12B the finished further alternative body care product according to the invention with the application unit and with a handle unit, in a schematic illustration,

FIG. 13A a finished further alternative body care product according to the invention which is realized as a flosser, with an application unit and with a handle unit having a hollow, in a schematic illustration,

FIG. 13B a schematic sectional view through the handle unit of the further alternative body care product according to the invention, along the section line A-A,

FIG. 14A a finished further alternative body care product according to the invention with an application unit and with a handle unit, in a schematic illustration,

FIG. 14B the finished further alternative body care product according to the invention with the application unit and with the handle unit, in a schematic side view in a put-up state,

FIG. 15A a finished further alternative body care product according to the invention with an application unit and with a handle unit, in a schematic illustration,

FIG. 15B the finished further alternative body care product according to the invention with the application unit and with the handle unit, in a schematic side view in a put-up state,

FIG. 16A a first layer of a handle unit of a further alternative body care product according to the invention which is realized as a flosser, during production, in a schematic illustration,

FIG. 16B a second layer of the further alternative body care products according to the invention, which forms an application unit and partially forms the handle unit, during production, in a schematic illustration,

FIG. 16C the finished further alternative body care product according to the invention with the application unit and with a handle unit, in a schematic illustration,

FIG. 17A an application unit of a further alternative body care product according to the invention which is realized as an interdental brush, during production, in a schematic illustration,

FIG. 17B a handle unit of the further alternative body care product according to the invention during production, in a schematic illustration,

FIG. 17C the finished further alternative body care product according to the invention with an application unit and with the handle unit, in a schematic illustration,

FIG. 18 a further alternative body care product according to the invention realized as a toothpick, with a first possible implementation of an application unit, in a schematic illustration,

FIG. 19 a further alternative body care product according to the invention realized as a toothpick, with a second possible implementation of an application unit, in a schematic illustration,

FIG. 20 a further alternative body care product according to the invention realized as a toothpick, with a third possible implementation of an application unit, in a schematic illustration,

FIG. 21A a bristle platelet of a head region of a further alternative body care product according to the invention which is realized as a toothbrush, in a schematic illustration,

FIG. 21B the bristle platelet of the head region of the further alternative body care product according to the invention which is realized as a toothbrush, in a state when furnished with bristles, in a schematic illustration,

FIG. 21C a handle unit of the further alternative body care product according to the invention which is realized as a toothbrush, during production, in a schematic illustration,

FIG. 21D the finished further alternative body care product according to the invention with an application unit comprising the bristle platelet and with the handle unit, in a schematic illustration,

FIG. 22A a further alternative body care product according to the invention which is realized as a flosser, with an application unit and with a two-part handle unit having crownings, in a schematic exploded view,

FIG. 22B the finished further alternative body care product according to the invention which is realized as a flosser, with the application unit and with the handle unit, in a schematic illustration,

FIG. 23 a finished further alternative body care product according to the invention which is realized as an interdental brush, with an application unit and with a handle unit, in a schematic illustration,

FIG. 24A a finished further alternative body care product according to the invention with an application unit, with a handle unit and with a protective element arranged at the handle unit, in a schematic illustration,

FIG. 24B the finished further alternative body care product according to the invention with the application unit and with the handle unit, in a schematic illustration with the protective element removed,

FIG. 25A a further alternative body care product according to the invention which is realized as a toothbrush, with a handle unit and with an application unit, in a non-folded state, in a schematic illustration,

FIG. 25B the further alternative body care product according to the invention in a folded application state with the handle unit and with the application unit, in a schematic 3D illustration,

FIG. 25C the further alternative body care product according to the invention in a folded application state with the handle unit and with the application unit, in a schematic 3D illustration,

FIG. 25D the further alternative body care product according to the invention in a folded application state with the handle unit and with the application unit, in a schematic side view,

FIG. 25E the further alternative body care product according to the invention in a folded application state with the handle unit and with the application unit, in a schematic view from below,

FIG. 26A a sales unit with a packaging and with a further alternative body care product according to the invention which is realized as a toothbrush, in a non-folded state, in a schematic illustration,

FIG. 26B the packaging of the sales unit in a folded, closed state, in a schematic 3D illustration,

FIG. 26C the further alternative body care product according to the invention in a folded application state with the handle unit and with the application unit, in a schematic 3D illustration from the front,

FIG. 26D the further alternative body care product according to the invention in the folded application state with the handle unit and with the application unit, in a schematic 3D illustration from the rear,

FIG. 27A layers of a handle unit of a further alternative body care product according to the invention which is realized as a toothbrush, during production, in a schematic illustration,

FIG. 27B the finished further alternative body care product according to the invention with an application unit comprising the bristle platelet and with the handle unit, in a schematic illustration from the front, and

FIG. 27C the finished further alternative body care product according to the invention with an application unit comprising the bristle platelet and with the handle unit, in a schematic illustration from the rear.

### DESCRIPTION OF THE EXEMPLARY EMBODIMENTS

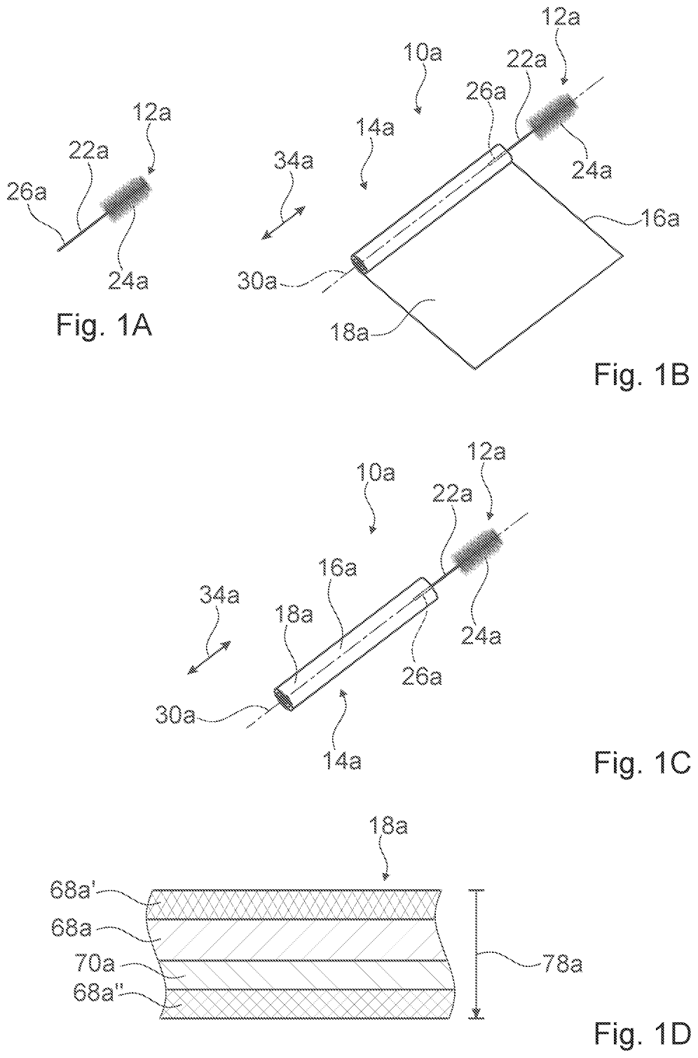

FIG. 1A shows an application unit 12a of a body care product 10a in a schematic perspective view. The body care product 10a is realized as an oral hygiene product. The body care product 10a is in the present case realized as an interdental brush. The body care product 10a may as well be realized as a one-way toothbrush or as an exchangeable-head toothbrush. The body care product 10a could also be realized as a flosser, as a single-tuft brush, as an interdental cleaner, as a tongue cleaner or the like. Furthermore, combined body care products 10a are conceivable which combine at least two different functions, for example a toothbrush with a tongue cleaner, a toothbrush with massaging elements, an interdental cleaner with a flosser, or more of the like.

The body care product 10a comprises the application unit 12a. The body care product 10a further comprises at least one handle unit 14a, which is connected with the application unit 12a.

In the following, FIGS. 1A to 10 will be referred to, which show different views and production stages of the body care product 10a. Due to the different views, there are some elements which are not shown in all figures and are therefore not provided with reference numerals in all figures.

The body care product 10a has a longitudinal axis 30a and a height axis. The longitudinal axis 30a is arranged parallel to a main extension direction 34a of the body care product 10a.

The application unit 12a is arranged at the upper side of the body care product 10a. The application unit 12a forms an uppermost point of the body care product 10a.

The handle unit 14a is configured for a gripping by the operator. The handle unit 14a has a cylindrical basic shape. The handle unit 14a further has a material handle body 16a. The material handle body 16a forms a material volume of the handle unit 14a. The material handle body 16a has a cylindrical shape. The material handle body 16a of the handle unit 14a is at least partially, in particular at least to a large extent, made of a paper material. The material handle body 16a is completely made of a paper material. The material handle body 16a of the handle unit 14a comprises at least one layer 18a of paper material, which is realized in a rolled-up fashion. The material handle body 16a exemplarily comprises precisely one layer 18a, however, a multi-layer structure would also be conceivable. The layer 18a of the paper material is rolled around a defined rolling axis. The rolling axis extends parallel to, in particular coaxially with, the longitudinal axis 30a of the body care product 10a. The layer 18a is rolled up during a production of the material handle body 16a. In a final state, in particular after production, the layer 18a forms a roll, in particular a cylindrical roll. The layer 18a of the material handle body 16a is rolled to form a cylindrical shape. The layer 18a of the material handle body 16a is connected to itself within the roll by gluing, welding and/or laminating. The paper material is rolled to form a massive roll. The material handle body 16a may in particular be rolled straight or in a spirally shape. The material handle body 16a has a diameter of 2 mm to 10 mm, preferably of 3 mm to 7 mm. Optionally, in addition to the tube of paper material, the material handle body 16a may also have a plastic head which, for example, prevents water from entering.

The application unit 12a further comprises a cleaning element carrier 22a and several, in particular twisted-in, cleaning elements 24a which are connected with the cleaning element carrier 22a. The cleaning element carrier 22a is exemplarily implemented by a wire, the cleaning elements 24a being formed as bristles which are twisted in the wire. However, a different implementation of the cleaning element carrier 22a and/or of the cleaning elements 24a, deemed expedient by someone skilled in the art, would also be conceivable. For a connection to the handle unit 14a, the cleaning element carrier 22a is connected to the material handle body 16a in a connection section 26a facing away from the cleaning elements 24a. The connection section 26a is in particular realized as a wire end facing away from the cleaning elements 24a. In particular in a finished state of the body care product 10a, the connection section 26a of the cleaning element carrier 22a of the application unit 12a protrudes into the material handle body 16a and is enclosed by the material handle body 16a. The application unit 12a is connected directly to the material handle body 16a via the connection section 26a. The cleaning element carrier 22a of the application unit 12a is implemented so as to be rolled in the layer 18a of the material handle body 16a at the connection section 26a that faces away from the cleaning elements 24a. Herein the connection section 26a of the application unit 12a is rolled into the material handle body 16a during production of the material handle body 16a. The connection section 26a of the application unit 12a is herein connected, like in particular glued, with the material handle body 16a directly during rolling of the layer 18a. The cleaning element carrier 22a of the application unit 12a is connected to the material handle body 16a by substance-to-substance bond, in particular by gluing and/or welding.

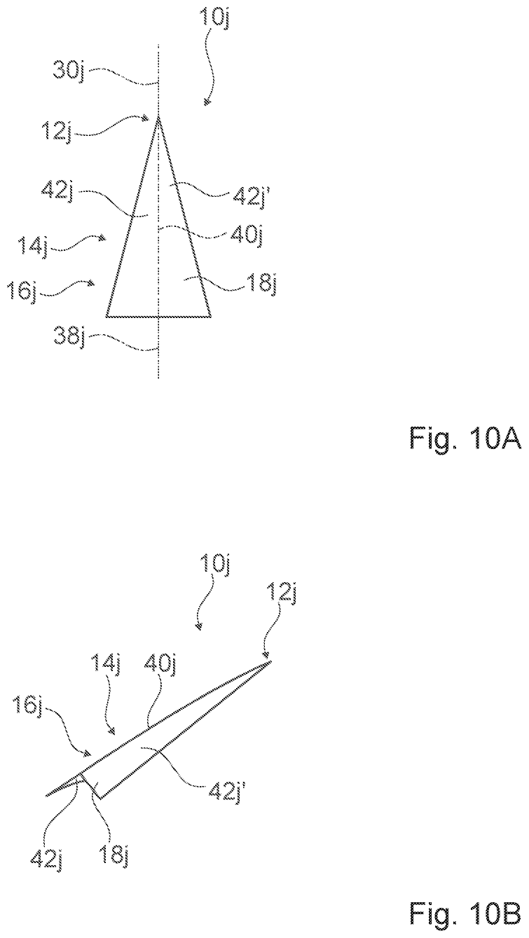

In particular, a method for producing the body care product 10a is disclosed. In the method the material handle body 16a of the handle unit 14a is rolled from the layer 18a that is made of a paper material. Furthermore, the application unit 12a is rolled into the material handle body 16a in the at least one connection section 26a. Herein in one variant a corresponding process flow for a production may be realized such that in a first step the application unit 12a is manufactured as an interdental brush by twisting-in (cf. FIG. 1A). Then in a second method step the at least one layer 18a of the material handle body 16a is rolled and at the same time the connection section 26a of the application unit 12a is rolled into the material handle body 16a (cf. FIG. 1B). An anchoring of the application unit 12a in the material handle body 16a may be realized in particular by glue, by welding or by pressing. For this a further method step may be required. A finished body care product 10a is shown in FIG. 10.

It is further provided that the paper material of the material handle body 16a has a water-repellent coating and/or impregnation. By a suitable coating and/or impregnation a reduction of the water intake of the paper material is achievable. The paper material may thus be suitable for multiple use as well. An impregnation may herein be realized, for example, by a plastic and/or a wax or the like. It is moreover provided that the paper material of the material handle body 16a is implemented so as to be varnished and/or printed. In particular, upgrading is attainable. An upgrading may, for example, be a varnishing, wherein for example a UV high-gloss varnish, a structure lacquer, a soft-touch varnish or a glitter varnish may be used. Alternatively or additionally, the paper material may have a printing. The paper material may, for example, have a printing of water-based varnishes, which are ecological with respect to conventional varnishes. Alternatively or additionally, it would also be conceivable that the ends of the material handle body 16a are pressed or are sealed, for example, with a wax. In the case of sealing, it would for example be conceivable that a filling material, like for example wax, is already provided on the paper material, such that only the ends of the material handle body 16a must be heated for a sealing.

FIG. 1D shows an exemplary structure of the layer 18a of the material handle body 16a. The layer 18a has several plies, respectively sub-layers 68a, 68a′, 68a″, which form the layer 18a. The layer 18a has a first ply, respectively sub-layer 68a, which forms a basic layer, in particular a carrier layer. The first ply, respectively sub-layer 68a, consists of a paper material, in particular a paper. The first ply, respectively sub-layer 68a, in particular has a printing 70a on an outer face. The printing 70a is in particular intended for a design of the material handle body 16a. The printing 70a is in particular applied onto the first ply, respectively sub-layer 68a, in a pre-treatment. The layer 18a further has a second ply, respectively sub-layer 68a′. The second ply, respectively sub-layer 68a′, is arranged on an inner face of the first ply, respectively sub-layer 68a. The second ply, respectively sub-layer 68a′, is implemented by a sealing wax. The second ply, respectively sub-layer 68a′, is applied onto the first ply, respectively sub-layer 68, in a pre-treatment. The sealing wax serves for a connection of the layer 18a in a rolling. The layer 18a further comprises a third ply, respectively sub-layer 68a″. The third ply, respectively sub-layer 68a″, is arranged on an outer side of the first ply, respectively sub-layer 68a. The third ply, respectively sub-layer 68a″, is realized by a protective varnish. The third ply, respectively sub-layer 68a″, is applied onto the first ply, respectively sub-layer 68a, in a pre-treatment. The protective varnish serves to protect the layer 18a from an ingress of humidity.

An arrow 78a in FIG. 1D exemplarily defines a direction from an inner face of the layer 18a to an outer face of the layer 18a.

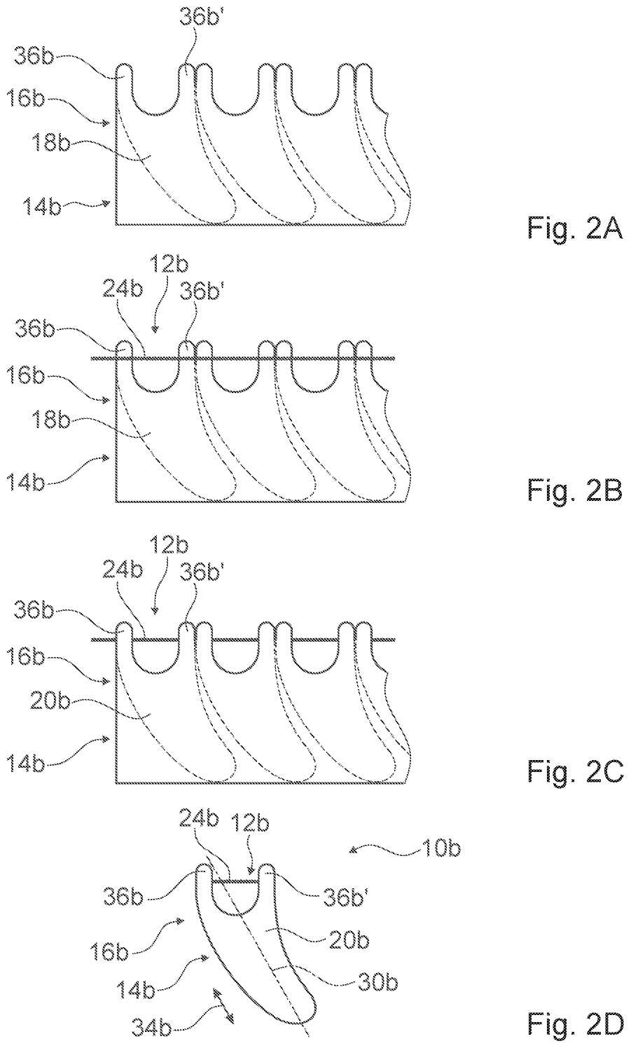

In FIGS. 2A to 27C further exemplary embodiments of the invention are shown. The following descriptions are limited essentially to the differences between the exemplary embodiments, wherein regarding components, features and functions which remain the same the description of the other exemplary embodiments, in particular of FIGS. 1A to 10, may be referred to. In order to distinguish between the exemplary embodiments, the letter a added to the reference numerals of the exemplary embodiment in FIGS. 1A to 10 has been replaced by the letters b to z and the ending aa in the reference numerals of the exemplary embodiments in FIGS. 2A to 27C. Regarding components having the same denomination, in particular regarding components having the same reference numerals, principally the drawings and/or the description of the other exemplary embodiments, in particular of FIGS. 1A to 1C, may be referred to.

FIG. 2A shows a first layer 18b of several handle units 14b of respectively one body care product 10b that is embodied as a flosser during production. In FIGS. 2A to 2D in particular several identical body care products 10b are partially illustrated, which are produced simultaneously. FIG. 2C herein shows a system with several identically implemented body care products 10b in each case having a material handle body 16b, wherein the material handle bodies 16b of the body care products 10b are connected to each other such that they are separable via perforations. The system in particular forms a sales unit. In the following only one of the body care products 10b will be referred to, a description being principally applicable to the further body care products 10b as well.

The body care product 10b comprises an application unit 12b. The body care product 10b further comprises at least one handle unit 14b which is connected to the application unit 12b.

The application unit 12b is arranged at an upper side of the body care product 10b. The application unit 12b forms an uppermost point of the body care product 10b. The application unit 12b comprises a cleaning element 24b, which is implemented by a dental floss. The application unit 12b in particular consists of the cleaning element 24b.

The handle unit 14b is configured for a gripping by the operator. The handle unit 14b further comprises a material handle body 16b. The material handle body 16b implements a material volume of the handle unit 14b. The material handle body 16b has a curved, flat basic shape split up in two arms 36b, 36b′ towards the application unit 12b. The arms 36b, 36b′ are configured to hold the dental floss on both sides. The material handle body 16b of the handle unit 14b is made at least partially, in particular at least to a large extent, of a paper material. The material handle body 16b is completely made of a paper material. The material handle body 16b of the handle unit 14b comprises at least two layers 18b, 20b of a paper material, which are connected to each other in a layered manner. The material handle body 16b of the handle unit 14b exemplarily comprises precisely two layers 18b, 20b of a paper material. Preferably two to six, preferably two to four layers 18b, 20b are provided. Preferentially the at least two layers 18b, 20b are arranged one above the other one perpendicularly to a main extension plane of the layers 18b, 20b and are connected to one another. The layers 18b, 20b are realized substantially identically and in particular form opposite-situated outer sides of the body care product 10b. The layers 18b, 20b are connected to each other, for example, by gluing and/or pressing and/or welding and/or laminating. The layers 18b, 20b are in particular connected to each other during a production of the body care product 10b.

The application unit 12b is arranged at the handle unit 14b between the at least two layers 18b, 20b of the material handle body 16b. The application unit 12b is arranged and fixed between the two layers 18b, 20b of the material handle body 16b in the region of the two arms 36b, 36b′. The cleaning element 24b of the application unit 12b spans between the arms 36b, 36b′.

In particular, a method for producing the body care product 10b is disclosed. In the method the material handle body 16b of the handle unit 14b is layered from the at least two interconnected layers 18b, 20b of paper material. In a production method, in particular sheets of paper material are pre-punched, which form the layers 18b, 20b. A pre-punching is implemented at least in the region where the application unit 12b is arranged. In particular, a pre-punching of such regions of the material handle body 16b is carried out, which can no longer be punched after a mounting of the application unit 12b. After this, in particular the pre-punched sheet that forms the first layer 18b is laid out and the cleaning element 24b is laid in (cf. FIGS. 2A and 2B). By way of example, the sheet is herein provided for a simultaneous production of several body care products 10b. Then a further pre-punched sheet, which forms the second layer 20b, is laid on and the layers 18b, 20b are connected, like for example by laminating, welding and/or pasting (cf. FIG. 2C). Following this the body care product 10b, in particular its outer geometry, can be subjected to final punching, and surplus dental floss thread of the application unit 12b can be removed (cf. FIG. 2D). It would also be conceivable that such a step is dispensed with and, instead of pre-punching, punching is executed directly, wherein the dental floss thread of the application unit 12b directly does not protrude beyond the final outer geometry. In at least one connection section 26b, the application unit 12b is glued and/or laminated in the material handle body 16b in between the at least two layers 18b, 20b. The cleaning element 24b of the application unit 12b may be anchored in a variety of manners which are deemed expedient by someone skilled in the art. An anchoring may be realized, for example, by a fastening of the cleaning element 24b of the application unit 12b by means of an adhesive material. The cleaning element 24b is exemplarily connected to the material handle body 16b by two parallel fixings. The cleaning element 24b embodied by a thread is moreover realized in the connection section 26b in a partially melted manner (not shown). The cleaning element 24b is connected to the material handle body 16b in parallel by gluing, in particular by means of a hot-setting glue, and by partial melting. The cleaning element 24b is herein melted by heat when exiting the sealed region. Due to the melting, a mushroom-shaped/bulb-shaped end is formed, which increases the pull-out force. That means that there is less tear-out of the cleaning element 24b in case of a load. The mushroom-shaped/bulb-shaped peripheral end of the cleaning element 24b has a larger diameter than the thread and thus abuts on the paper material on the outside. Alternatively or additionally, an anchoring may be realized by welding. The cleaning element 24b of the application unit 12b is fixable between the layers 18b, 20b by welding. An individual finished body care product 10b is shown in FIG. 2D.

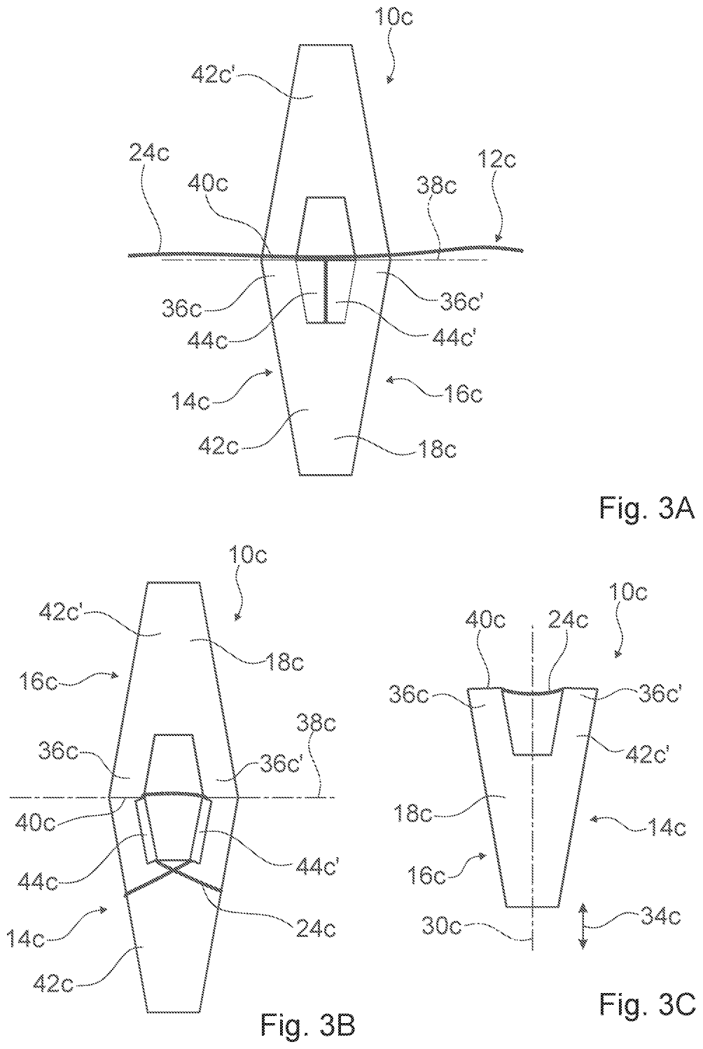

FIG. 3A shows a body care product 10c, which is embodied as a flosser, in a non-folded state during production. The body care product 10c comprises the application unit 12c. The body care product 10c further comprises at least one handle unit 14c, which is connected with the application unit 12c. The application unit 12c is arranged at an upper side of the body care product 10c. The application unit 12c constitutes an uppermost point of the body care product 10c. The application unit 12c comprises a cleaning element 24c, which is realized by a dental floss. The application unit 12c in particular consists of the cleaning element 24c.

The handle unit 14c is configured for a gripping by the operator. The handle unit 14c further comprises a material handle body 16c. The material handle body 16c implements a material volume of the handle unit 14c. In a final state the material handle body 16c has a flat basic shape split into two arms 36c, 36c′ towards the application unit 12c. The arms 36c, 36c′ are configured to hold the dental floss on both sides. The material handle body 16c of the handle unit 14c is at least partially, in particular to a large extent, made of a paper material. The material handle body 16c is completely made of a paper material. The material handle body 16c of the handle unit 14c comprises at least one layer 18c of paper material, which is realized so as to be at least partially folded. By way of example, the material handle body 16c of the handle unit 14c comprises precisely one layer 18c of paper material. The layer 18c has a folding axis 38c. In the folding axis 38c the layer 18c has a folding edge 40c. In a usage state, the layer 18c is folded around the folding axis 38c at the folding edge 40c. The layer 18c is preferably realized so as to be perforated and/or embossed in the folding edge 40c in order to permit defined folding. The layer 18c is divided into two sides, in particular sub-layers 42c, 42c′, by the folding edge 40c. A second sub-layer 42c′ of the sub-layers 42c, 42c′ is folded onto a first sub-layer 42c of the sub-layers 42c, 42c′ so as to create several layers. For this the material handle body 16c has on top of the arms 36c, 36c′ the folding edge 40c, which is embodied as a hinge. In the folding edge 40c the layer 18c is foldable by 180°. The folding axis 38c of the layer 18c extends perpendicularly to the main extension direction 34c of the body care product 10c. The material handle body 16c may herein in particular be punched from paper sheets or off a reel. Furthermore, the sub-layers 42c, 42c′ of the material handle body 16c are connected by sealing or gluing. The layer 18c of the material handle body 16c is cut or punched.

The application unit 12c is anchored at tension tabs 44c, 44c′ of the material handle body 16c. The tension tabs 44c, 44c′ are connected integrally with the first sub-layer 42c of the layer 18c. The tension tabs 44c, 44c′ project from the arms 36c, 36c′ inwards and are configured to be bent. The cleaning element 24c of the application unit 24c is guided around the tension tabs 44c, 44c′. In this way tearing-out is preventable. Furthermore, the cleaning element 24c of the application unit 12c may also be fixed in another point, for example by an adhesive dot. Alternatively, the cleaning element 24c of the application unit 12c may be formed as a ring or as a noose, i. e. in particular as a closed ring having no end, such that a further fixing in addition to the tension tabs 44c, 44c′ can be dispensed with. The ring that is made from the thread may herein be produced directly as a ring or may be produced by knotting. During production, the paper material of the material handle body 16c is in particular sealed, wherein the tension tabs 44c, 44c′ are sealed in, thus fixing the cleaning element 24c.

In particular, a method is disclosed for producing the body care product 10c. In the method the material handle body 16c of the handle unit 14c is layered from the at least two interconnected layers 18c, 20c of paper material. In a production method, in particular the layer 18c is punched and the application unit 12c is provided (cf. FIG. 3A). Then the tension tabs 44c, 44c′ are bent and the cleaning element 24c of the application unit 12c is guided around the tension tabs 44c, 44c′ (cf. FIG. 3B). Furthermore, the cleaning element 24c of the application unit 12c may then be fixed in another point as well, for example by means of an adhesive dot. After this, surplus dental floss thread of the application unit 12c can be removed. Alternatively, the cleaning element 24c of the application unit 12c may also be formed as a ring or as a noose, i. e. in particular as a closed ring having no end, such that a further fixing, in addition to the tension tabs 44c, 44c′, may be dispensed with. Following this, the second sub-layer 42c′ of the sub-layers 42c, 42c′ is folded and sealed onto the first sub-layer 42c, wherein the tension tabs 44c, 44c′ are sealed in. A finished body care product 10c is shown in FIG. 3C.

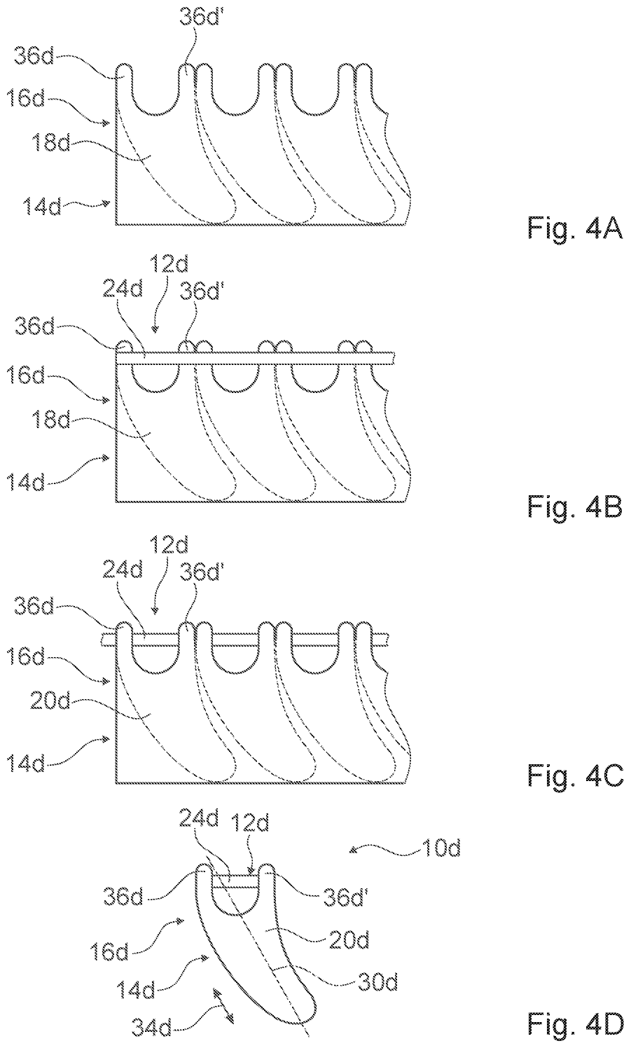

FIG. 4A shows a first layer 18d of several handle units 14d of respectively one body care product 10d embodied as a flosser during production. In FIGS. 4A to 4D in particular several identical body care products 10d are partially shown, which are produced simultaneously. Herein FIG. 4C shows a system with several body care products 10d, which are implemented identically and in each case have a material handle body 16d, wherein the material handle bodies 16d of the body care products 10d are connected to each other such that they are separable via perforations. The system in particular constitutes a sales unit. In the following, essentially only one of the body care products 10d will be referred to, a description being principally applicable to the further body care products 10d as well.

The body care product 10d comprises an application unit 12d. The body care product 10d further comprises a handle unit 14d, which is connected with the application unit 12d.

The application unit 12d is arranged at the upper side of the body care product 10d. The application unit 12d forms an uppermost point of the body care product 10d. The application unit 12d is implemented at least partially, in particular at least to a large extent, of a paper material. The application unit 12d comprises a cleaning element 24d comprising a paper material. The cleaning element 24d is embodied as an interdental cleaning paper. The application unit 12d consists of the cleaning element 24d.

The handle unit 14d is configured for a gripping by the operator. The handle unit 14d further comprises a material handle body 16d. The material handle body 16d implements a material volume of the handle unit 14d. The material handle body 16d has a curved, flat basic shape, which is split in two arms 36d, 36d′ towards the application unit 12d. The arms 36d, 36d′ are configured to hold the cleaning element 24d on both sides. The material handle body 16d of the handle unit 14d is implemented at least partially, in particular at least to a large extent, of a paper material. The material handle body 16d is implemented completely of a paper material. The material handle body 16d of the handle unit 14d comprises at least two layers 18d, 20d of a paper material, which are connected to each other in a layered manner. The material handle body 16d of the handle unit 12d exemplarily comprises precisely two layers 18d, 20d of a paper material. Preferably two to six, preferably two to four, layers 18d, 20d are provided. Preferentially the at least two layers 18d, 20d are arranged above one another perpendicularly to a main extension plane of the layers 18d, 20d and are connected to one another. The layers 18d, 20d are realized substantially identically, and in particular form opposite-situated outer sides of the body care product 10d. The layers 18d, 20d are connected to each other, for example, by gluing and/or pressing and/or welding and/or laminating. The layers 18d, 20d are in particular connected to each other during a production of the body care product 10d.

The application unit 12d is arranged at the handle unit 14d between the at least two layers 18d, 20d of the material handle body 16d. The application unit 12d is arranged and fixed between the two layers 18d, 20d of the material handle body 16d in the region of the two arms 36d, 36d′. The application unit 12d is spanned between the arms 36d, 36d′.

In particular, a method is disclosed for producing the body care product 10d. In the method the material handle body 16d of the handle unit 14d is layered from the at least two interconnected layers 18d, 20d of a paper material. In a production method, in particular sheets of paper material are pre-punched, which form the layers 18d, 20d. A pre-punching is carried out at least in the region where the application unit 12d is arranged. In particular, a pre-punching of such regions of the material handle body 16d is realized which can be punched no longer after a mounting of the application unit 12d. Then in particular the pre-punched sheet which forms the first layer 18d is laid out and the cleaning element 24d is laid therein (cf. FIGS. 4A and 4B). Herein, by way of example, the sheet is configured for a simultaneous production of several body care products 10d. Following this, a further pre-punched sheet, which forms the second layer 20d, is laid on and the layers 18d, 20d are connected, for example by laminating, welding and/or gluing (cf. FIG. 4C). After this the body care product 10d, in particular its outer geometry, can subjected to final punching and surplus cleaning element 24d of the application unit 12d can be removed (cf. FIG. 4D). It would also be conceivable that such a step is dispensed with and, instead of pre-punching, punching is carried out directly and the cleaning element 24d of the application unit 12d directly does not project beyond the final outer geometry. The application unit 12d is glued and/or laminated in the material handle body 16d in between the at least two layers 18d, 20d in at least one connection section 26d. The cleaning element 24d of the application unit 12d may be anchored in different manners deemed expedient by someone skilled in the art. An anchoring may be realized, for example, by fastening the cleaning element 24d of the application unit 12d by means of an adhesive material. Alternatively or additionally, an anchoring may be realized by welding. The dental floss thread of the application unit 12d may be fastened between the layers 18d, 20d by welding. An individual finished body care product 10d is shown in FIG. 4D.

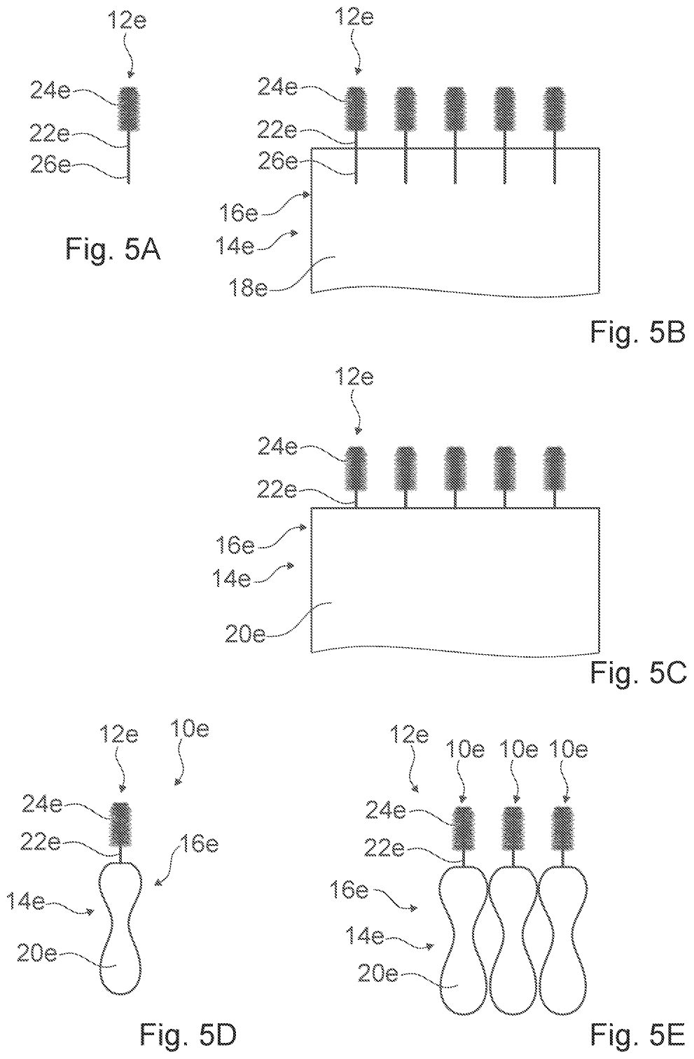

FIG. 5A shows an application unit 12e of a body care product 10e in a schematic illustration. The body care product 10e is in the present case embodied as an interdental brush. In FIGS. 5A to 5C and 5E in particular several identical body care products 10e are partially shown, which are produced simultaneously. In the following, essentially only one of the body care products 10e will be referred to, a description being principally also applicable to the further body care products 10e.

The body care product 10e comprises the application unit 12e. The body care product 10e further comprises at least one handle unit 12e, which is connected with the application unit 12e.

The application unit 12e is arranged at the upper side of the body care product 10e. The application unit 12e forms an uppermost point of the body care product 10e.

The handle unit 14e is configured for a gripping by the operator. The handle unit 14e further comprises a material handle body 16e. The material handle body 16e implements a material volume of the handle unit 14e. The material handle body 16e has a flat elongate basic shape. The material handle body 16e of the handle unit 14e is at least partially, preferably at least to a large extent, made of a paper material. The material handle body 16e is completely made of a paper material. The material handle body 16e of the handle unit 14e comprises at least two layers 18e, 20e of paper material, which are connected to each other in a layered fashion. The material handle body 16e of the handle unit 14e exemplarily comprises precisely two layers 18e, 20e of a paper material. Preferably two to six, preferably two to four layers 18e, 20e are provided. Preferentially the at least two layers 18e, 20e are arranged one above the other one perpendicularly to a main extension plane of the layers 18e, 20e and are connected to one another. The layers 18e, 20e are realized substantially identically and in particular form opposite-situated outer sides of the body care product 10e. The layers 18e, 20e are connected to each other, for example, by gluing and/or pressing and/or welding and/or laminating. The layers 18e, 20e are in particular connected to each other during a production of the body care product 10e.

The application unit 12e further comprises a cleaning element carrier 22e and several, in particular twisted-in, cleaning elements 24e, which are connected with the cleaning element carrier 22e. By way of example, the cleaning element carrier 22e is implemented by a wire, wherein the cleaning elements 24e are embodied as bristles which are twisted in with the wire.

for a connection to the handle unit 14e, the cleaning element carrier 22e is connected to the material handle body 16e in a connection section 26e which faces away from the cleaning elements 24e. The connection section 26e is in particular implemented by a wire end facing away from the cleaning elements 24e. In particular in a state when the body care product 10e has been manufactured, the connection section 26e of the cleaning element carrier 26e of the application unit 12e protrudes into the material handle body 16e and is enclosed by the material handle body 16e. The application unit 12e is connected directly to the material handle body 16e via the connection section 26e. The connection section 26e is arranged at the handle unit 14e between the at least two layers 18e, 20e of the material handle body 16e. Furthermore, for a partially form-fit connection to the handle unit 14e, the cleaning element carrier 22e of the application unit 12e is implemented in a bent fashion (not visible) in the connection section 26e. The connection section 26e of the cleaning element carrier 22e has an angled structure for an anchoring in the handle body 16e.

In particular, a method is disclosed for producing the body care product 10e. In the method the material handle body 16e of the handle unit 14e is layered from the at least two interconnected layers 18e, 20e of paper material. A respective process flow for a production could herein be realized in a variant such that in a first step the application unit 12e is produced as an interdental brush by twisting-in (cf. FIG. 5A). Then in particular a sheet which forms the first layer 18e is laid out, and the application unit 12e is laid therein with the connection section 26e (cf. FIG. 5B). The sheet is herein exemplarily configured for a simultaneous production of several body care products 10e. Following this, a further sheet, which forms the second layer 20e, is laid on and the layers 18e, 20e are connected, for example by laminating, welding and/or gluing (cf. FIG. 5C). After this the body care product 10e, in particular its outer geometry, can be punched (cf. FIG. 5D). An individual finished body care product 10e is shown in FIG. 5D.

Optionally, in a production of the body care products 10e a grouping of the body care products 10e may be realized, which are separated for usage. The body care products 10e may be partly connected via the paper material, there being in particular a perforation (not visible in detail), at which the body care products 10e can be separated. FIG. 5E shows a system with several identically implemented body care products 10e which in each case comprise a material handle body 16e, the material handle bodies 16e of the body care products 10e being connected to each other such that they are separable via perforations. This in particular allows simplified processing, wherein a production is realized in groups and separation does not occur until usage (cf. FIG. 5E).

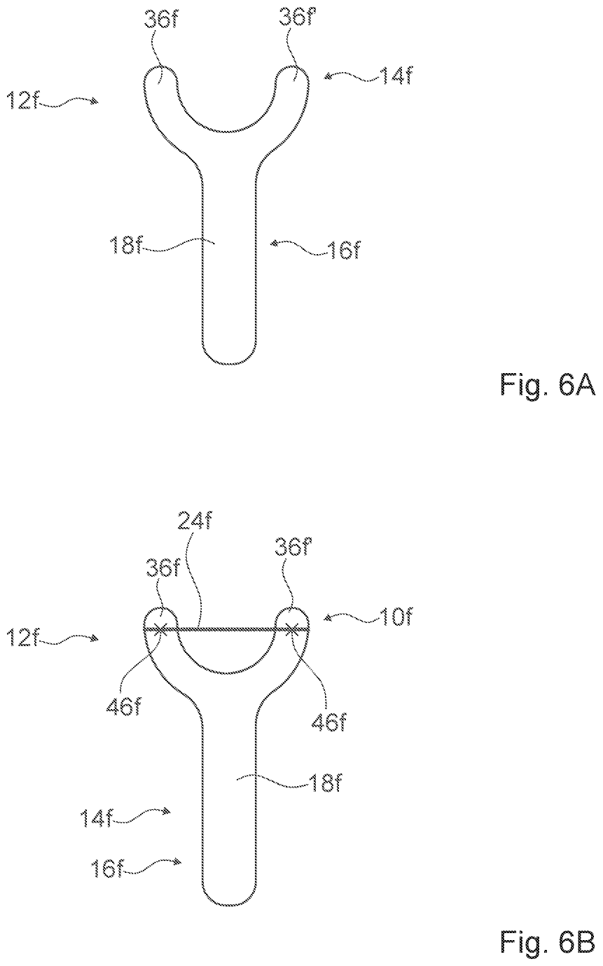

FIG. 6A shows a layer 18f of a handle unit 14f of a body care product 10f, which is realized as a flosser, during production. The body care product 10f comprises an application unit 12f. The body care product 10f further comprises at least one handle unit 14f, which is connected with the handle unit 12f. The application unit 12f is arranged at the upper side of the body care product 10f. The application unit 12f constitutes an uppermost point of the body care product 10f. The application unit 12f comprises a cleaning element 24f, which is implemented by a dental floss. The application unit 12f in particular consists of the cleaning element 24f.

The handle unit 14f is configured for a gripping by the operator. The handle unit 14f further comprises a material handle body 16f. The material handle body 16f implements a material volume of the handle unit 14f. In a final state, the material handle body 16f has a flat basic shape split into two arms 36f, 36f′ towards the application unit 12f. The arms 36f, 36f′ are configured to hold the cleaning element 24f on both sides. The material handle body 16f of the handle unit 14f is made at least partially, in particular at least to a large extent, of a paper material. The material handle body 16f is made completely of a paper material. The material handle body 16f of the handle unit 14f comprises at least one layer 18f of a paper material. The layer 18f of the material handle body 16f is cut or punched.

The application unit 12f is sewed with the material handle body 16f. The cleaning element 24f is on both ends sewed with the material handle body 16f at the ends of the arms 36f, 36f′. The body care product 10f comprises further threads 46f, by means of which the cleaning element 24f is sewed to the material handle body 16f. It would also be conceivable that the dental floss thread is sewed onto the first layer 18f, the dental floss thread being used as a sewing means. The layer 18f has a material thickness of 0.5 mm to 2 mm, preferably of 0.5 mm to 1.5 mm, with a density of 180 g/m² to 500 g/m², preferably 240 g/m² to 400 g/m², in particular so as to enable a connection by sewing.

In particular, a method is disclosed for producing the body care product 10f. In the method the application unit 12f is sewed onto the material handle body 16f. In a production method in particular the layer 18f is punched (cf. FIG. 6A). In particular, at least such regions of the material handle body 16f are punched in which the application unit 12f is arranged. Then the layer 18f is laid out and the cleaning element 24f is provided and is sewed on by means of the threads 46f, 46f′ (cf. FIG. 6B). A finished body care product 10f is shown in FIG. 6B.

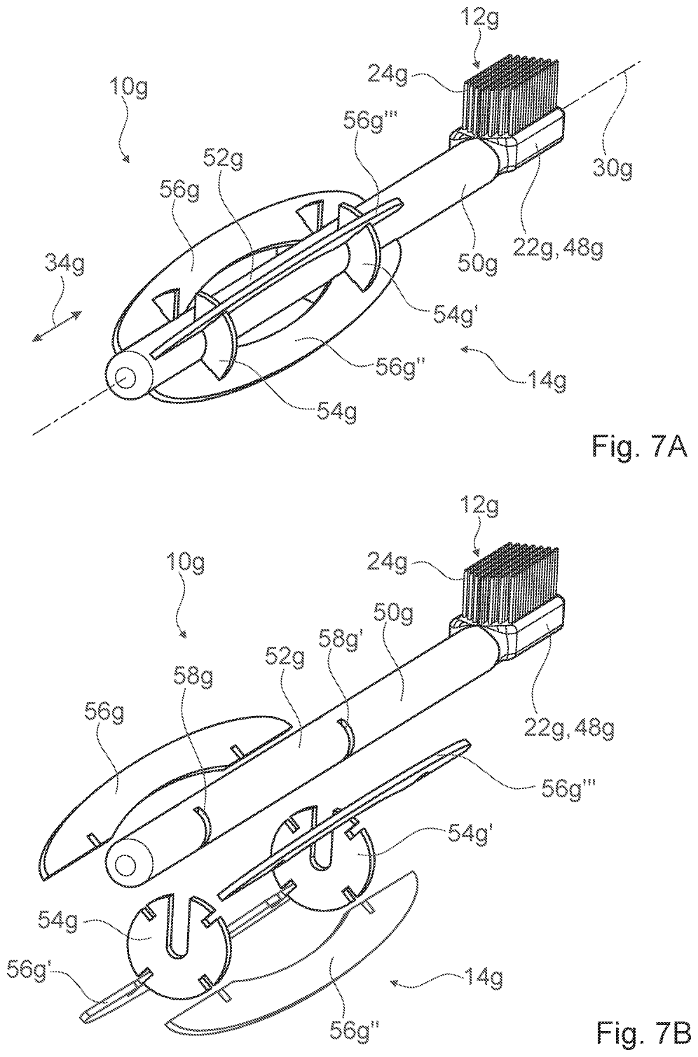

FIG. 7A shows a front side of a body care product 10g in a schematic perspective illustration. The body care product 10g is in the present case embodied as a toothbrush.

The body care product 10g comprises at least one application unit 12g. The body care product 10g further comprises at least one handle unit 14g.

The at least one application unit 12g comprises a head region 48g. The head region 48g forms a brush head of the body care product 10g. The head region 48g forms a cleaning element carrier 22g of the body care product 10g. In the present case the head region 48g is embodied as a toothbrush head. The head region 48g in particular comprises a base body which is realized as a bristle carrier. The base body of the head region 48g is completely made of a hard component. However, it would also be conceivable that the base body of the head region 48g is implemented of a hard component and a soft component. The base body of the head region 48g forms a bristle carrier. The body care product 10g further comprises in the head region 48g a plurality of bristle holes realized as recesses, which in each case receive one bristle bundle. The bristle bundles forms cleaning elements 24g of the body care product 10g.

Alternatively, the body care product 10g could have a bristle platelet, which is accommodated in the head region 48g and comprises a plurality of bristle bundles. The bristle platelet is exemplarily embodied as an AFT platelet.

Any suitable bristles may be used as bristles. base body 52g. In the present case the application unit 12g further comprises a neck region 50g, which in particular connects the head region 48g to the handle unit 14g. The neck region 50g forms a neck.

The body care product 10g further comprises a base body 52g. The base body 52g consists at least partially of a hard component. The base body 52g consists completely of a hard component. The base body 52g is realized in a one-part implementation. The base body 52g forms the application unit 12g. The base body 52g forms the application unit 12g completely. The base body 52g furthermore forms the application unit 12g partially. The base body 52g forms a core of the handle unit 14g. The base body 52g has in a region of the handle unit 14g an at least approximately cylindrical basic shape. The base body 52g extends along a longitudinal axis 30g along the entire body care product 10g. At a rear end of the body care product 10g, the base body 52g has a rounded end. The base body 52g may be made of a paper material.

The handle unit 14g is configured for a gripping by the operator. The handle unit 14g has a cylindrical basic shape. The handle unit 14g further comprises a material handle body 16g. The material handle body 16g implements a material volume of the handle unit 14g. The material handle body 16g has an ellipsoid shape. The material handle body 16g of the handle unit 14g is at least partially, in particular at least to a large extent, made of a paper material. The material handle body 16g is completely made of a paper material. The material handle body 16g of the handle unit 14g comprises several sub-elements 54g, 54g′, 56g, 56g′, 56g″, 56g′″, which are configured to be plugged together. For this purpose, in particular a paper material is cut into the sub-elements 54g, 54g′, 56g, 56g′, 56g″, 56g′″ and then the sub-elements 54g, 54g′, 56g, 56g′, 56g″, 56g′″ are plugged together.

The base body 52g and the handle body 16g realize a form-fit connection. The sub-elements 54g, 54g′, 56g, 56g′, 56g″, 56g′″ of the material handle body 16g are plugged to the base body 52g. For this the base body 52g has form-fitting recesses 58g, 58g′, which are configured for a form-fit connection to at least two sub-elements 54g, 54g′ of the handle body 16g. The first two sub-elements 54g, 54g′ are implemented as disks of paper material, which in each case have a receiving groove for sliding onto the form-fit recesses 58g, 58g′. The first two sub-elements 54g, 54g′ furthermore have in each case circumferentially on an outer side form-fit recesses for a form-fit connection to the further sub-elements 56g, 56g′, 56g″, 56g′″ of the handle body 16g. The further sub-elements 56g, 56g′, 56g″, 56g′″ are in each case implemented by arc-shaped elements extending along the longitudinal axis 30g along the base body 52g. This in particular allows providing an advantageously three-dimensional handle unit 14g, wherein a material input can be kept low. The body care product 10g, respectively its body, may herein in particular consist of 2 to 25, preferably of 4 to 12, individual parts.

Plugging together of the body care product 10g may herein be realized already during production but also by a user himself. If the plugging together is realized by the user, it is in particular possible to keep a packaging size of the body care product 10g advantageously small (cf. FIG. 7B).

The body care product 10g is produced in a method. In the method, the base body 52g with the application unit 12g is produced and provided in a first method step in an injection-molding procedure. Then the sub-elements 54g, 54g′, 56g, 56g′, 56g″, 56g— are cut and optionally the sub-elements 54g, 54g′, 56g, 56g′, 56g″, 56g— are plugged to the base body 52g and plugged together. Possibly safeguarding may be provided for the sub-elements 54g, 54g′, 56g, 56g′, 56g″, 56g″.

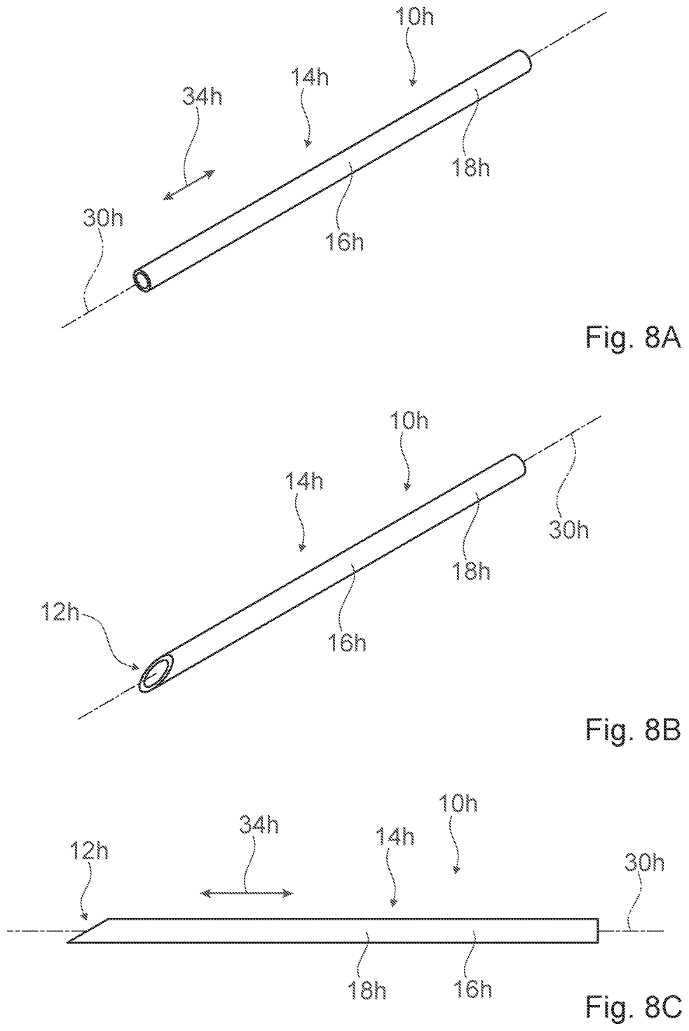

FIG. 8A shows a body care product 10h in a schematic perspective illustration. The body care product 10h is in the present case embodied as a toothpick.

The body care product 10h comprises an application unit 12h. The body care product 10h further comprises at least one handle unit 14h, which is connected to the application unit 12h.

The handle unit 14h is configured for a gripping by the operator. The handle unit 14h has a cylindrical basic shape. The handle unit 14h further comprises a material handle body 16h. The material handle body 16h implements a material volume of the handle unit 14h. The material handle body 16h has a cylindrical shape. The material handle body 16h of the handle unit 14h consists at least partially, in particular at least to a large extent, of a paper material. The material handle body 16h consists completely of a paper material. The material handle body 16h of the handle unit 14h comprises at least one layer 18h of paper material, which is realized in a rolled-up fashion. Exemplarily the material handle body 16h comprises precisely one layer 18h; however, a multi-layer structure would also be conceivable. The layer 18h of paper material is rolled up around a defined rolling axis. The rolling axis extends parallel to, in particular coaxially with, the longitudinal axis 30h of the body care product 10h. The layer 18h is rolled up during a production of the material handle body 16h. In a final state, in particular after production, the layer 18h forms a roll, in particular a cylindrical roll. The layer 18h of the material handle body 16h is rolled to form a cylindrical shape. The layer 18h of the material handle body 16h is connected to itself within the roll by pasting, welding and/or laminating. The paper material is rolled to form a hollow-cylindrical roll. The material handle body 16h may in particular be rolled up straight or in a spiral shape.

The application unit 12h is realized integrally with the handle unit 14h. The application unit 12h is implemented by obliquely cutting off one end of the material handle body 16h.

For a production of the body care product 10h herein in particular paper ribbons are wound off reels and are together formed into a tube. The paper ribbons are herein in particular rolled onto a core and are then connected on the core. Following this, one end of the tube may be cut obliquely and/or pointed, such that the application unit 12f is created at one end. The application unit 12h is herein in particular realized integrally with the handle unit 14f. Optionally it would be conceivable that a surface of the body care product 10h is subjected to post-treatment. It would herein be conceivable that the body care product 10h is varnished for reducing water intake or that the body care product 10h is printed.

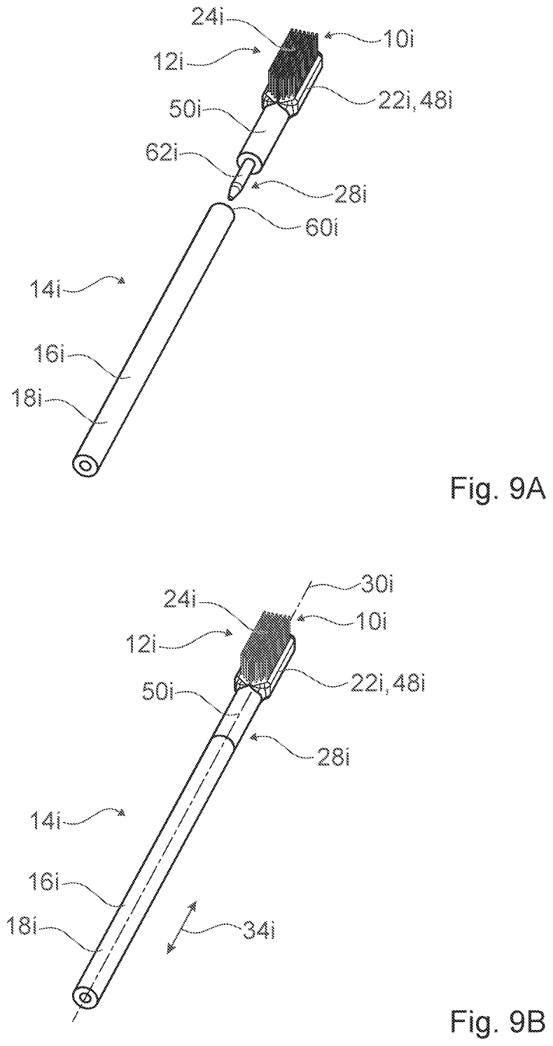

FIG. 9A shows a body care product 10i in a schematic perspective illustration. The body care product 10i is in the present case implemented as a toothbrush.

The body care product 10i comprises an application unit 12i. The body care product 10i further comprises at least one handle unit 14i, which is connected to the application unit 12i.

The application unit 12i is exemplarily realized as an exchangeable head for a toothbrush. The at least one application unit 12i comprises a head region 48i. The head region 48i forms a brush head of the body care product 10i. In the present case the head region 48i is embodied as a toothbrush head. The head region 48i in particular has a base body that is embodied as a bristle carrier. The base body of the head region 48i is completely made of a hard component. However, it would also be conceivable that the base body of the head region 48i is made of a hard component and a soft component. The base body of the head region 48i forms a bristle carrier. In the head region 48i the body care product 10i furthermore has a plurality of bristle holes, which are realized as recesses and in each case accommodate one bristle bundle.

Alternatively, the body care product 10i could comprise a bristle platelet, which is accommodated in the head region 48i and comprises a plurality of bristle bundles. The bristle platelet is exemplarily embodied as an AFT platelet.

Any suitable bristles may be used as bristles. In the present case the application unit 12i further comprises a neck region 50i, which in particular connects the head region 48i to the handle unit 14i. The neck region 50i forms a neck.

The handle unit 14i is configured for a gripping by the operator. The handle unit 14i has a cylindrical basic shape. The handle unit 14i further comprises a material handle body 16i. The material handle body 16i implements a material volume of the handle unit 14i. The material handle body 16i has a cylindrical shape. The material handle body 16i of the handle unit 14i is made at least partially, in particular at least to a large extent, of a paper material. The material handle body 16i is completely made of a paper material. The material handle body 16i of the handle unit 14i comprises at least one layer 18i of a paper material, which is realized in a rolled-up fashion. The material handle body 16i exemplarily comprises precisely one layer 18i; however, a multi-layer structure is also conceivable. The layer 18i of a paper material is rolled around a defined rolling axis. The rolling axis extends parallel to, in particular coaxially with, a longitudinal axis 30i of the body care product 10i. The layer 18i is rolled up during a production of the material handle body 16i. In a final state, in particular after production, the layer 18i forms a roll, in particular a cylindrical roll. The layer 18i of the material handle body 16i is rolled into a cylindrical shape. The layer 18i of the material handle body 16i is connected to itself within the roll by gluing, welding and/or laminating. The paper material is rolled to form a hollow-cylindrical roll. The material handle body 16i may in particular be rolled straight or in a spiral shape.

The body care product 10i further comprises a plug connection unit 28i, which is configured for a releasable plugging connection of the application unit 12i, which is in particular embodied as a brush head, to the handle unit 14i. The plug connection unit 28i is realized at least partially integrally with the application unit 12i and at least partially integrally with the handle unit 14i. The plug connection unit 28i comprises a first plug connection element 60i, which is integrally connected with the handle unit 14i, and a second plug connection element 62i, which correspond to the first plug connection element 60i and which is fixedly connected with the application unit 12i. The first plug connection element 60i is realized as a cylindrical recess in the material handle body 16i. The recess may herein be produced already by a hollow-cylindrical manufacturing of the handle body 16i or by subsequent drilling. The second plug connection element 62i is realized by a pin-shaped projection, which is configured to engage in the first plug connection element 60i. In particular, the application unit 12i is separable from the material handle body 16i by means of the plug connection unit 28i. The plug connection unit 28i is in particular configured to provide a releasable or non-releasable latch connection.

FIG. 10A shows a body care product 10j in a non-folded state during production. The body care product 10j comprises an application unit 12j. The body care product 10j further comprises a handle unit 14j, which is connected to the application unit 12j.

The handle unit 14j is configured for a gripping by the operator. The handle unit 14j further comprises a material handle body 16j. The material handle body 16j implements a material volume of the handle unit 14j. In a final state the material handle body 16j has a flat triangular basic shape. The material handle body 16j of the handle unit 14j is at least partially, in particular at least to a large extent, made of a paper material. The material handle body 16j is completely made of a paper material. The material handle body 16j of the handle unit 14j comprises at least one layer 18j of a paper material, which is in a usage state realized so as to be at least partially folded. The material handle body 16j of the handle unit 14j exemplarily comprises precisely one layer 18j of a paper material. The layer 18i has a folding axis 38j. In the folding axis 38j the layer 18j has a folding edge 40j. The layer 18j is in a usage state folded at the folding edge 40j around the folding axis 38j. The layer 18j is at the folding edge 40j implemented so as to be perforated, fluted and/or embossed in order to enable defined folding. The folding axis 38j of the layer 18j is parallel to the main extension direction 34j of the body care product 10j. The material handle body 16j may herein in particular be folded from paper sheets or from a reel.

The application unit 12j is realized integrally with the handle unit 14j. The application unit 12j is realized by folding a pointed end of the material handle body 16j. The body care product 10j may in a usage in particular be folded to form a triangle (cf. FIG. 10B).

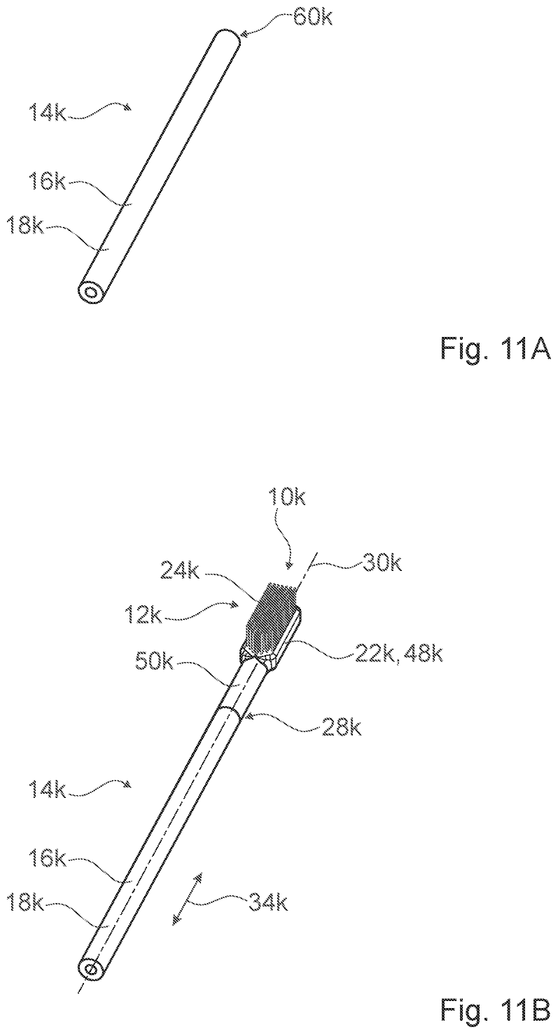

FIG. 11A shows a handle unit 14k of a body care product 10k in a schematic perspective illustration. The body care product 10k is in the present case embodied as a toothbrush.

The body care product 10k comprises an application unit 12k. The body care product 10k further comprises the handle unit 14k, which is connected to the application unit 12k.

The application unit 12k is produced in an injection-molding procedure. The at least one application unit 12k comprises a head region 48k. The head region 48k forms a brush head of the body care product 10k. In the present case the head region 48k is embodied as a toothbrush head. The head region 48k in particular has a base body which is embodied as a bristle carrier. The base body of the head region 48k is completely made of a hard component. However, it would also be conceivable that the base body of the head region 48k is made of a hard component and a soft component. The base body of the head region 48k forms a bristle carrier. The body care product 10k furthermore comprises in the head region 48k a plurality of bristle holes realized as recesses, which in each case accommodate one bristle bundle.

Alternatively, the body care product 10k could comprise a bristle platelet, which is accommodated in the head region 48k and comprises a plurality of bristle bundles. The bristle platelet is exemplarily embodied as an AFT platelet.

Any suitable bristles may be used as bristles. In the present case the application unit 12k further comprises a neck region 50k, which in particular connects the head region 48k to the handle unit 14k. The neck region 50k forms a neck.

The handle unit 14k is configured for a gripping by an operator. The handle unit 14k has a cylindrical basic shape. The handle unit 14k further comprises a material handle body 16k. The material handle body 16k implements a material volume of the handle unit 14k. The material handle body 16k has a cylindrical shape. The material handle body 16k of the handle unit 14k is made at least partially, in particular at least to a large extent, of a paper material. The material handle body 16k is made completely of a paper material. The material handle body 16k of the handle unit 14k comprises at least one layer 18k of a paper material, which is realized in a rolled-up fashion. The material handle body 16k exemplarily comprises precisely one layer 18k; however, a multi-layer structure would also be conceivable. The layer 18k of the paper material is rolled around a defined rolling axis. The rolling axis extends parallel to, in particular coaxially with, the longitudinal axis 30k of the body care product 10k. The layer 18k is rolled up during a production of the material handle body 16k. In a final state, in particular after production, the layer 18k forms a roll, in particular a cylindrical roll. The layer 18k of the material handle body 16k is rolled to form a cylindrical shape. The layer 18k of the material handle body 16k is connected to itself within the roll by gluing, welding and/or laminating. The paper material is rolled to form a hollow-cylindrical roll. The material handle body 16k may in particular be rolled straight or in a spiral shape.

The application unit 12k is injection-molded to the handle unit 14k by means of an injection-molding procedure. For this the handle unit 14k is in particular laid into an injection-molding tool for the application unit 12, wherein the application unit 12k is produced directly at the handle unit 14k. A connection of the overmolding to the paper material may herein in particular be achieved by undercuts, breakthroughs, i. e. in particular a form-fit connection, or chemically, like in particular by a material bond. For this purpose, the paper material may in particular be realized so as to be pre-treated, in particular before overmolding, to enable the connection, for example pre-treated with special varnishes and/or coatings. FIG. 11B shows the finished body care product 10k.

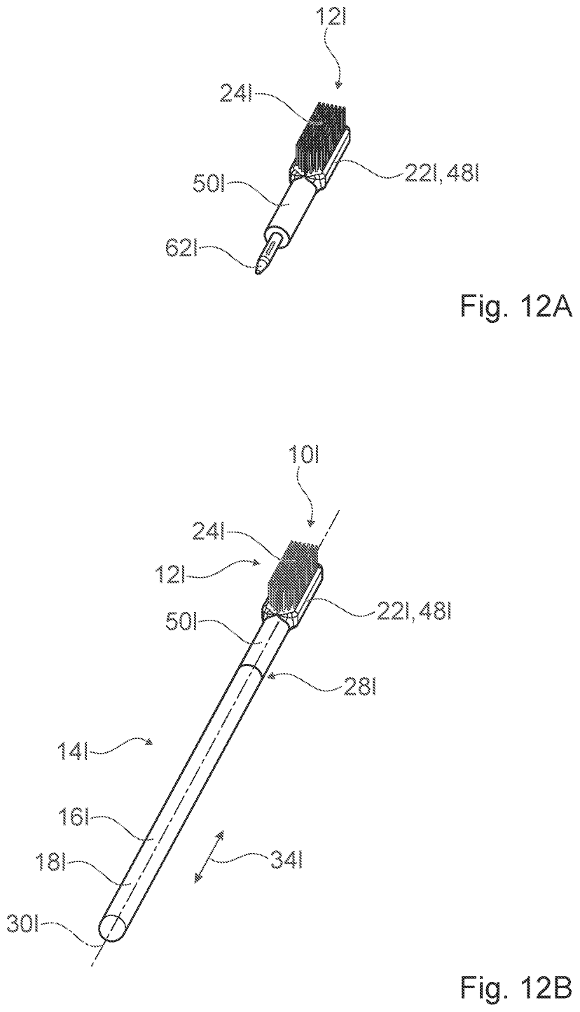

FIG. 12A shows an application unit 12l of a body care product 10l in a schematic perspective illustration. The body care product 10l is in the present case embodied as a toothbrush.

The body care product 10l comprises the application unit 12l. The body care product 10l further comprises at least one handle unit 14l, which is connected to the application unit 12l.

The application unit 12l is produced in a plastic injection-molding procedure. The at least one application unit 12l comprises a head region 48l. The head region 48l forms a brush head of the body care product 10l. In the present case the head region 48l is embodied as a toothbrush head. The head region 48l in particular has a base body which is embodied as a bristle carrier. The base body of the head region 48l is completely made of a hard component. However, it would also be conceivable that the base body of the head region 48l is made of a hard component and a soft component. The base body of the head region 48l forms a bristle carrier. The body care product 10l further comprises in the head region 48l a plurality of bristle holes realized as recesses, which in each case accommodate one bristle bundle.

Alternatively, the body care product 10l could comprise a bristle platelet, which is accommodated in the head region 48k and comprises a plurality of bristle bundles. The bristle platelet is exemplarily implemented as an AFT platelet.

Any suitable bristles may be used as bristles. In the present case the application unit 12l further comprises a neck region 50l, which in particular connects the head region 48l to the handle unit 14l. The neck region 50l forms a neck.

The handle unit 14l is configured for a gripping by the operator. The handle unit 14l has a cylindrical basic shape. The handle unit 14l further comprises a material handle body 16l. The material handle body 16l implements a material volume of the handle unit 14l. The material handle body 16l has a cylindrical shape. The material handle body 16l of the handle unit 14l is made at least partially, in particular at least to a large extent, of a paper material. The material handle body 16l is made completely of a paper material. The handle body 16l is cast from a paper material. The paper material is herein in particular processed in a manner similar to injection molding. The raw products are in particular composed of industrial starch, preferably from potatoes, paper fibers, water and an additive mix. The paper mixture created therefrom is then used for a further processing in by means of injection-molding technique. The paper injection molding is in particular realized in three steps. In a first step an injection is made in which the paper mixture is injected into an aluminum tool. In a second step a baking process is executed, in which the paper mixture is baked, in particular heated, in the tool. In a third step unforming is carried out, in which the finished body care product 10l can be removed.

The application unit 12l is overmolded with the paper material of the handle body 16l. The application unit 12l is produced following a known procedure, like in particular a plastic injection-molding procedure (cf. FIG. 12A). Herein a connection of the overmolding to the functional part may in particular be achieved by undercuts, breakthroughs, i. e. in particular a form-fit connection, or chemically, like in particular by a material bond. The application unit 12l comprises a core projection 64l which protrudes into the handle body 16l. The application unit 12l may in particular be realized so as to have been pre-treated, in particular before the overmolding, in order to enable the connection, for example pre-treated with special varnishes and/or coatings. Following the production of the application unit 12l, the handle unit 14l is molded to the application unit 12l in a paper injection-molding procedure. FIG. 12B shows the finished body care product 10l.

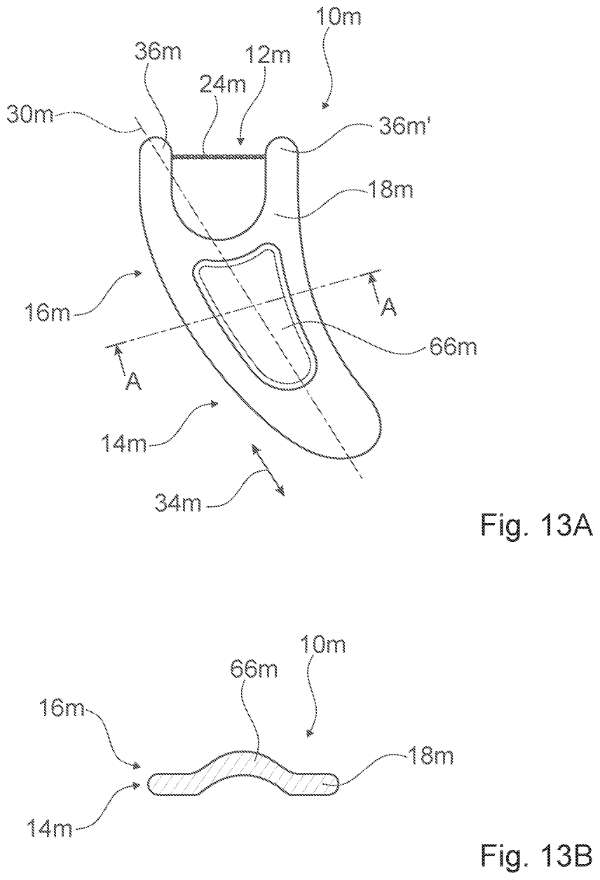

FIG. 13A shows a body care product 10m which is embodied as a flosser. The body care product 10m comprises an application unit 12m. The body care product 10m further comprises a handle unit 14m, which is connected to the application unit 12m. The application unit 12m is arranged at the upper side of the body care product 10m. The application unit 12m constitutes an uppermost point of the body care product 10m. The application unit 12m comprises a cleaning element 24m, which is embodied as a dental floss. The application unit 12m in particular consists of the cleaning element 24m.

The handle unit 14m is configured for a gripping by the operator. The handle unit 14m further comprises a material handle body 16m. The material handle body 16m implements a material volume of the handle unit 14m. In a final state, the material handle body 16m has a flat basic shape which splits into two arms 36m, 36m′ towards the application unit 12m. The arms 36m, 36m′ are configured to hold the cleaning element 24m on both sides. The material handle body 16m of the handle unit 14m is made at least partially, in particular at least to a large extent, of a paper material. The material handle body 16m is made completely of a paper material. The material handle body 16m of the handle unit 14m comprises at least one layer 18m of a paper material. The layer 18m of the material handle body 16m is cut or punched.

Furthermore, the layer 18m of the handle body 16m is deformed, preferably deep-drawn, in particular by moistening, pressure and heat. The material handle body 16m has an oval deformation 66m. The layer 18m has an embossing realized as the oval deformation 66m. Thus in particular a gripping geometry is created. It is in particular possible to provide a body care product 10m with a voluminous handle unit 14m.

The deformation may be implemented in different ways. On the one hand, the individual layers may be deformed before a connection, while on the other hand the layers may be deformed after a connection, as a whole. In the case of a deformation before a connection of the layers, it is crucial that the outer contour is correctly oriented with respect to the deformation 66m. This is to achieve that, if the layers have already been punched out when the connection takes place, identical products are formed at the outer contour without differences in the layers. On the other hand, if the products are not punched out until after the deformation, this is to ensure that the deformation is always situated in the same place. In the case of a deformation after the connection, it must be taken into account that the deformation process requires more effort due to the increased material thickness as there are more layers to be deformed.

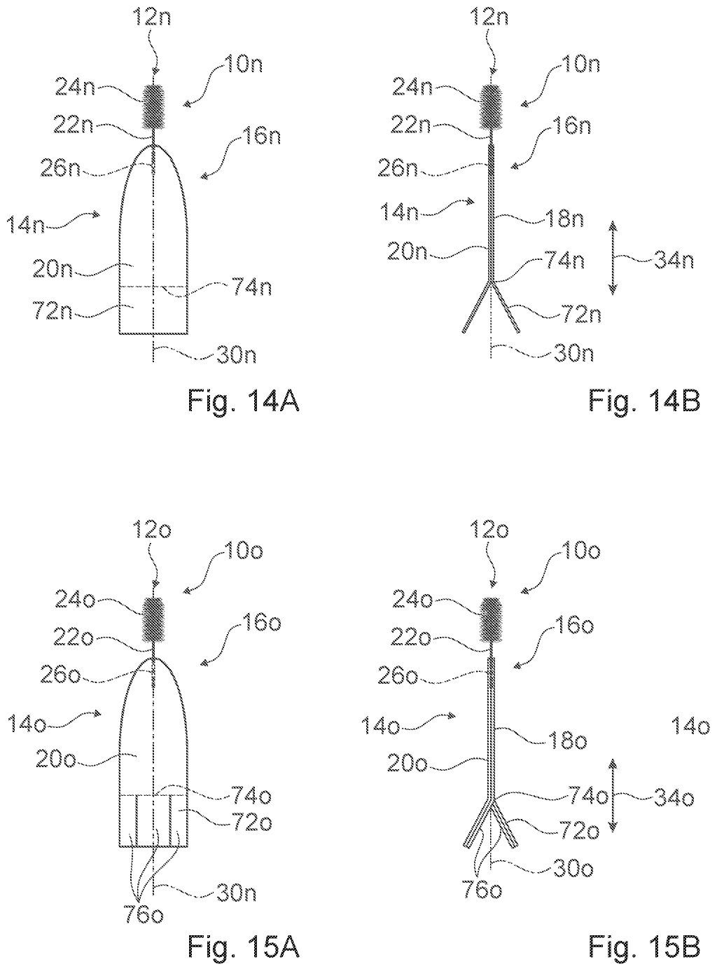

FIG. 14A shows an application unit 12n of a body care product 10n in a schematic illustration. The body care product 10n is in the present case embodied as an interdental brush.

The body care product 10n comprises the application unit 12n. The body care product 10n further comprises at least one handle unit 14n, which is connected to the application unit 12n.

The application unit 12n is arranged at the upper side of the body care product 10n. The application unit 12n forms an uppermost point of the body care product 10n.

The handle unit 14n is configured for a gripping by the operator. The handle unit 14n further comprises a material handle body 16n. The material handle body 16n implements a material volume of the handle unit 14n. The material handle body 16n has a flat elongate basic shape. The material handle body 16n of the handle unit 14n is at least partially, in particular at least to a large extent, made of a paper material. The material handle body 16n is completely made of a paper material. The material handle body 16n of the handle unit 14n comprises at least two layers 18n, 20n of a paper material, which are connected to each other in a layered fashion. The material handle body 16n of the handle unit 14n exemplarily comprises precisely two layers 18n, 20n of a paper material. Preferably two to six, preferably two to four layers 18n, 20n are provided. Preferentially the at least two layers 18n, 20n are arranged one above the other one perpendicularly to a main extension plane of the layers 18n, 20n and are connected to one another. The layers 18n, 20n are realized substantially identically and in particular form opposite-situated outer sides of the body care product 10n. The layers 18n, 20n are connected to each other, for example, by gluing and/or pressing and/or welding and/or laminating. The layers 18n, 20n are connected to each other in particular during a production of the body care product 10n.

The application unit 12n further comprises a cleaning element carrier 22n and several, in particular twisted-in, cleaning elements 24n, which are connected with the cleaning element carrier 22n. The cleaning element carrier 22n is exemplarily implemented as a wire, the cleaning elements 24n being bristles which are twisted into the wire.

For a connection to the handle unit 12n, the cleaning element carrier 22n is connected to the material handle body 16n in a connection section 26n that faces away from the cleaning elements 24n. The connection section 26n is in particular realized as a wire end that faces away from the cleaning elements 24n. In particular in a state when the body care product 10n has been completed, the connection section 26n of the cleaning element carrier 22n of the application unit 12n projects into the material handle body 16n and is enclosed by the material handle body 16n. The application unit 12n is connected directly to the material handle body 16n via the connection section 26n. The connection section 26n is arranged at the handle unit 14n between the at least two layers 18n, 20n of the material handle body 16n. Furthermore, for a partially form-fit connection to the handle unit 14n, the cleaning element carrier 22n of the application unit 12n is realized so as to be bent in the connection section 26n (not visible).

The handle unit 14n further forms a base foot 72n. The two layers 18n, 20n are for this purpose realized so as to be unconnected in a lower section that faces away from the application unit 12n. The base foot 72n is implemented by folding the two layers 18n, 20n apart in the lower section. In particular, folding edges 74n are provided, at which the layers 18n, 20n can be folded away from each other in the lower section (cf. FIG. 14B). This may additionally be used for a complete separation of the layers 18n, 20n in order to separate the body care product 10n from the paper material on a material side for disposal. The paper material is then disposable in the paper collection and the application unit 12n is separately disposable in the household waste.

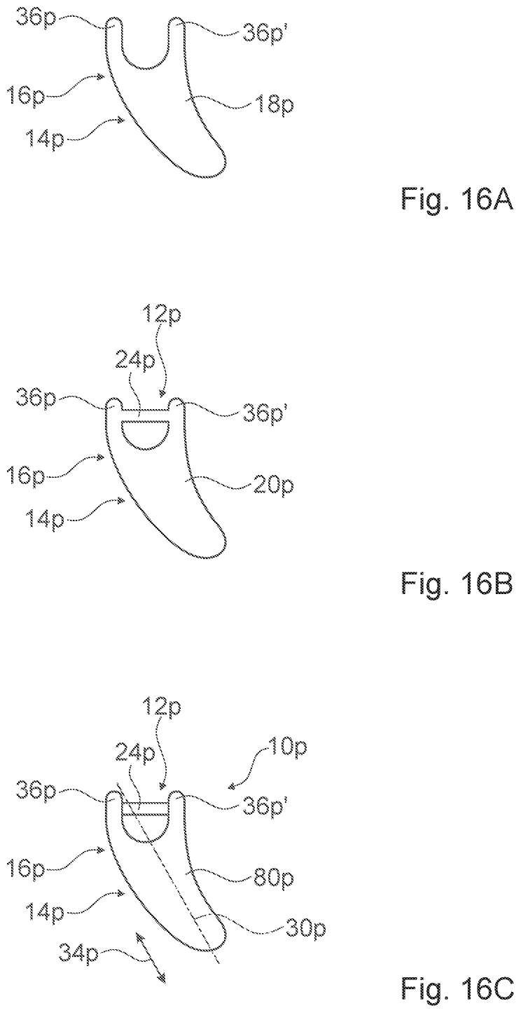

FIG. 15A shows an application unit 120 of a body care product 10o in a schematic illustration. The body care product 10o is in the present case embodied as an interdental brush.

The body care product 10o comprises the application unit 120. The body care product 100 further comprises at least one handle unit 140, which is connected to the application unit 120.

The application unit 12o is arranged at an upper side of the body care product 100. The application unit 12o constitutes an uppermost point of the body care product 100.

The handle unit 14o is configured for a gripping by the operator. The handle unit 140 further comprises a material handle body 160. The material handle body 16o implements a material volume of the handle unit 140. The material handle body 16o has a flat elongate basic shape. The material handle body 160 of the handle unit 14o is at least partially, in particular at least to a large extent, made of a paper material. The material handle body 16o is completely made of a paper material. The material handle body 160 of the handle unit 14o comprises at least two layers 180, 200 of a paper material, which are connected to each other in a layered fashion. The material handle body 160 of the handle unit 14o exemplarily comprises precisely two layers 180, 200 of a paper material. Preferably two to six, preferably two to four, layers 180, 20o are provided. Preferentially the at least two layers 180, 20o are arranged one above the other one perpendicularly to a main extension plane of the layers 180, 20o and are connected to one another. The layers 180, 20o are realized substantially identically and in particular form opposite-situated outer sides of the body care product 100. The layers 180, 20o are, for example, connected to each other by gluing and/or pressing and/or welding and/or laminating. The layers 180, 20o are in particular connected to each other during a production of the body care product 100.

The application unit 12o moreover comprises a cleaning element carrier 22o and several cleaning elements 24o which are connected, in particular twisted-in, with the cleaning element carrier 22o. The cleaning element carrier 22o is exemplarily realized as a wire, the cleaning elements 24o being implemented as bristles which are twisted into the wire.

For a connection to the handle unit 140, the cleaning element carrier 22o is connected to the material handle body 16o in the connection section 26o which faces away from the cleaning elements 24o. The connection section 26o is in particular implemented as a wire end facing away from the cleaning elements 24o. In particular in a state when the body care product 10o has been completed, the connection section 26o of the cleaning element carrier 22o of the application unit 12o projects into the material handle body 16o and is enclosed by the material handle body 160. The application unit 12o is connected directly to the material handle body 16o via the connection section 26o. The connection section 26o is arranged at the handle unit 14o between the at least two layers 180, 200 of the material handle body 160. Furthermore, for a partially form-fit connection to the handle unit 140, the cleaning element carrier 22o of the application unit 12o is realized so as to be bent in the connection section 26o (not visible).

The handle unit 14o furthermore forms a base foot 720. The two layers 180, 20o are for this purpose implemented so as to be slitted parallel to a main extension direction 34o in a lower section that faces away from the application unit 120. The handle unit 140 exemplarily has two slits in the lower section, which extend through both layers 180, 200. It would also be conceivable that a perforation is provided instead of the slits, which is separated by an end customer to form a slit. The handle unit 14o thus forms three projections 76o in the lower section. The base foot 72o is created by way of folding the projections 76o apart such that they oppose one another. In particular, a folding edge 740 is provided, at which the projections 76o can be folded apart, respectively bent apart, in the lower section.

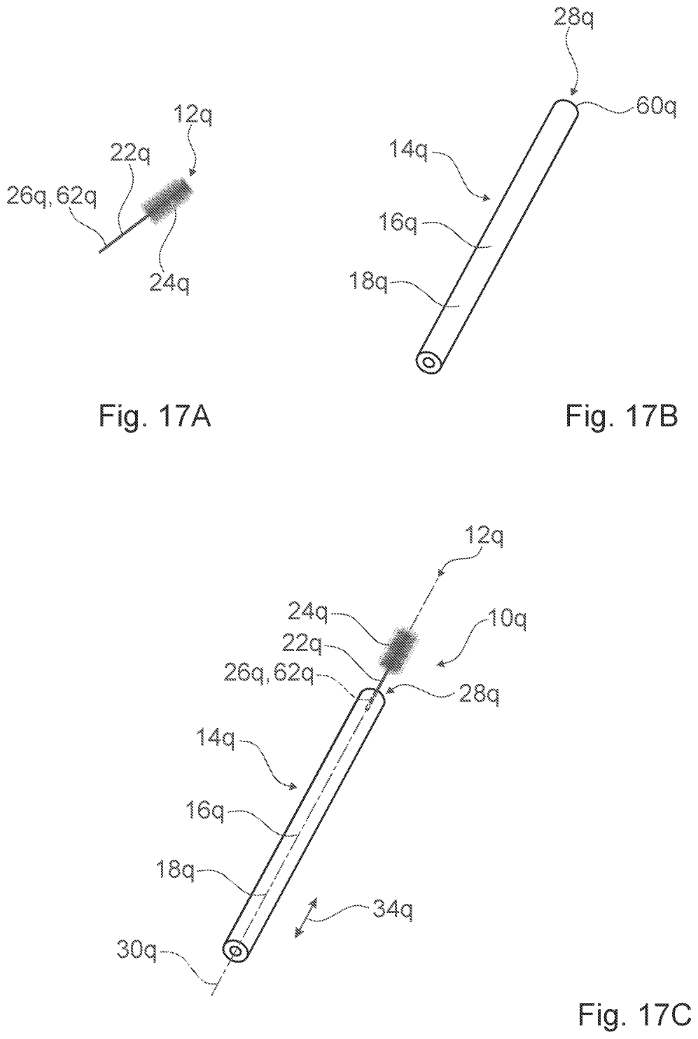

FIG. 16A shows a first layer 18p of a handle unit 14p of a body care product 10p, which is embodied as a flosser, during production. The body care product 10p comprises an application unit 12p. The body care product 10p further comprises a handle unit 14p, which is connected to the application unit 12p.

The application unit 12p is arranged at the upper side of the body care product 10p. The application unit 12p forms an uppermost point of the body care product 10p. The application unit 12p is at least partially, in particular at least to a large extent, made of a paper material. The application unit 12p has a cleaning element 24p comprising a paper material. The cleaning element 24p is realized as an interdental cleaning paper. The application unit 12p consists of the cleaning element 24p.

The handle unit 14p is configured for a gripping by the operator. The handle unit 14p further comprises a material handle body 16p. The material handle body 16p implements a material volume of the handle unit 14p. The material handle body 16p has a curved flat basic shape, which is split into two arms 36p, 36p′ towards the application unit 12p. The arms 36p, 36p′ are configured to hold the cleaning element 24p on both sides. The material handle body 16p of the handle unit 14p is at least partially, in particular at least to a large extent, made of a paper material. The material handle body 16p is completely made of a paper material. The material handle body 16p of the handle unit 14p comprises at least three layers 18p, 20p, 80p of a paper material, which are connected to one another in a layered fashion. The material handle body 16p of the handle unit 14p exemplarily comprises precisely three layers 18p, 20p, 80p, which are at least partially made of a paper material, wherein a second layer 20p additionally forms the application unit 12p. Preferentially the at least three layers 18p, 20p, 80p are arranged above one another perpendicularly to a main extension plane of the layers 18p, 20p, 80p and are connected to one another. The first and third layers 18p, 80p are realized substantially identically and in particular form opposite-situated outer sides of the body care product 10p. The second layer 20p is arranged between the first layer 18p and the third layer 80p. The layers 18p, 20p, 80p are connected, for example, by gluing and/or pressing and/or welding and/or laminating. The layers 18p, 20p, 80p are in particular connected to one another during a production of the body care product 10p.

The second layer 20p additionally forms the application unit 12p. The second layer 20p is, at least in a region of the application unit 12p, made of a film. Preferably the second layer 20p is completely made of a film.

In particular, a method for producing the body care product 10p is disclosed. In the method the body care product 10p is layered from the three interconnected layers 18p, 20p, 80p, which are at least partially made of a paper material. In a production method, in particular sheets of paper material and/or film, which form the layers 18p, 20p, 80p, are pre-punched, in particular completely punched. Then in particular the pre-punched sheets, which form the layers 18p, 20p, 80p, are laid out and connected, like for example by laminating, welding and/or gluing.

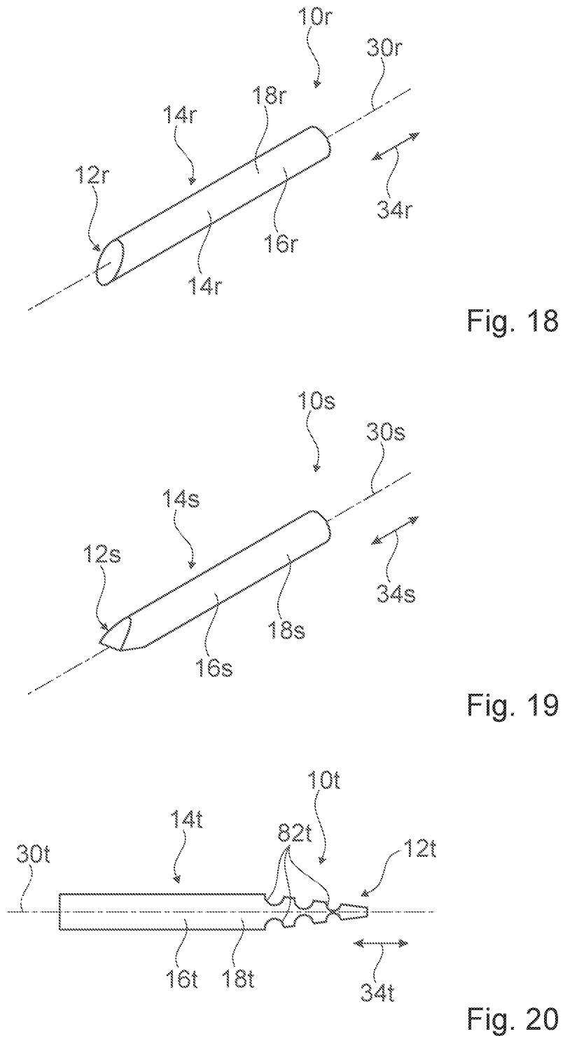

FIG. 17A shows a body care product 10q in a schematic perspective illustration. The body care product 10q is in the present case embodied as an interdental brush.

The body care product 10q comprises an application unit 12q. The body care product 10q further comprises at least one handle unit 14q, which is connected to the application unit 12q.

The handle unit 14q is configured for a gripping by the operator. The handle unit 14q has a cylindrical basic shape. The handle unit 14q further comprises a material handle body 16q. The material handle body 16q implements a material volume of the handle unit 14q. The material handle body 16q has a cylindrical shape. The material handle body 16q of the handle unit 14q is at least partially, in particular at least to a large extent, made of a paper material. The material handle body 16q is completely made of a paper material. The material handle body 16q of the handle unit 14q comprises at least one layer 18q of a paper material, which is realized in a rolled-up fashion. The material handle body 16q exemplarily comprises precisely one layer 18q; however, a multi-layer structure would also be conceivable. The layer 18q of paper material is rolled around a defined rolling axis. The rolling axis extends parallel to, in particular coaxially with, a longitudinal axis 30q of the body care product 10q. The layer 18q is rolled up during a production of the material handle body 16q. In a final state, in particular after production, the layer 18q forms a roll, in particular a cylindrical roll. The layer 18q of the material handle body 16q is rolled to form a cylindrical, in particular hollow-cylindrical, shape. The layer 18q of the material handle body 16q is connected in itself within the roll by gluing, welding and/or laminating. The paper material is rolled to form a hollow-cylindrical roll. The material handle body 16q may in particular be rolled straight or in a spiral shape.

Beyond this, the application unit 12q comprises a cleaning element carrier 22q and several cleaning elements 24q, which are connected, in particular twisted-in, with the cleaning element carrier 22q. The cleaning element carrier 22q is exemplarily implemented by a wire, wherein the cleaning elements 24q are realized as bristles which are twisted into the wire. However, a different implementation of the cleaning element carrier 22q and/or the cleaning elements 24q, deemed expedient by someone skilled in the art, would also be conceivable. For a connection to the handle unit 14q, the cleaning element carrier 22q is connected to the material handle body 16q in a connection section 26q which faces away from the cleaning elements 24q. The connection section 26q is in particular implemented by a wire end facing away from the cleaning elements 24q.

The body care product 10q further comprises a plug connection unit 28q configured for a releasable plug connection of the application unit 12q, which is in particular embodied as an interdental brush, to the handle unit 14q. The plug connection unit 28q is realized at least partially integrally with the application unit 12q and at least partially integrally with the handle unit 14q. The plug connection unit 28q comprises a first plug connection element 60q, which is integrally connected with the handle unit 14q, and a second plug connection element 62q, which corresponds to the first plug connection element 60q and is fixedly connected with the application unit 12q. The second plug connection element 62q is embodied by the connection section 26q. The first plug connection element 60q is embodied as a cylindrical recess in the material handle body 16q. Herein the recess may be already manufactured by a hollow-cylindrical production of the handle body 16q or by subsequent drilling. In particular, the application unit 12q is separable from the material handle body 16q by means of the plug connection unit 28q. The plug connection unit 28q is in particular configured to provide a releasable or non-releasable latch connection.

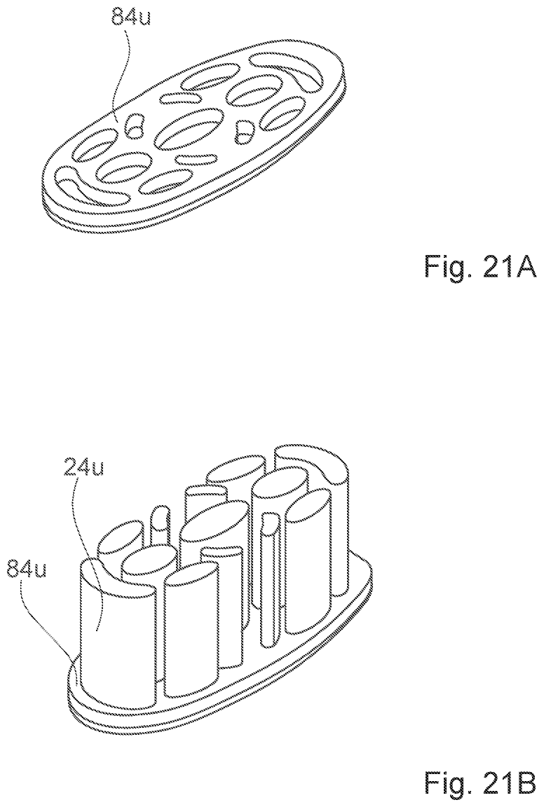

FIG. 18 shows a body care product 10r in a schematic perspective illustration. The body care product 10r is in the present case embodied as a toothpick.

The body care product 10r comprises an application unit 12r. The body care product 10r further comprises at least one handle unit 14r, which is connected to the application unit 12r.

The handle unit 14r is configured for a gripping by the operator. The handle unit 14r has a cylindrical, in particular solid-cylindrical, basic shape. The handle unit 14r further comprises a material handle body 16r. The material handle body 16r implements a material volume of the handle unit 14r. The material handle body 16r has a cylindrical shape. The material handle body 16r of the handle unit 14r is at least partially, in particular at least to a large extent, made of a paper material. The material handle body 16r is completely made of a paper material. The material handle body 16r of the handle unit 14r comprises at least one layer 18r of a paper material, which is realized in a rolled-up fashion. The layer 18r of paper material is rolled around a defined rolling axis. The rolling axis extends parallel to, in particular coaxially with, a longitudinal axis 30r of the body care product 10r. The layer 18r is rolled up during a production of the material handle body 16r. In a final state, in particular after production, the layer 18r forms a roll, in particular a cylindrical roll. The layer 18r of the material handle body 16r is rolled to form a solid-cylindrical shape. The layer 18r of the material handle body 16r is connected in itself within the roll by gluing, welding and/or laminating. The paper material is rolled to form a solid-cylindrical roll. The material handle body 16r may in particular be rolled up straight or in a spiral shape.

The application unit 12r is implemented integrally with the handle unit 14r. The application unit 12r is realized by oblique cutting off an end of the material handle body 16r.

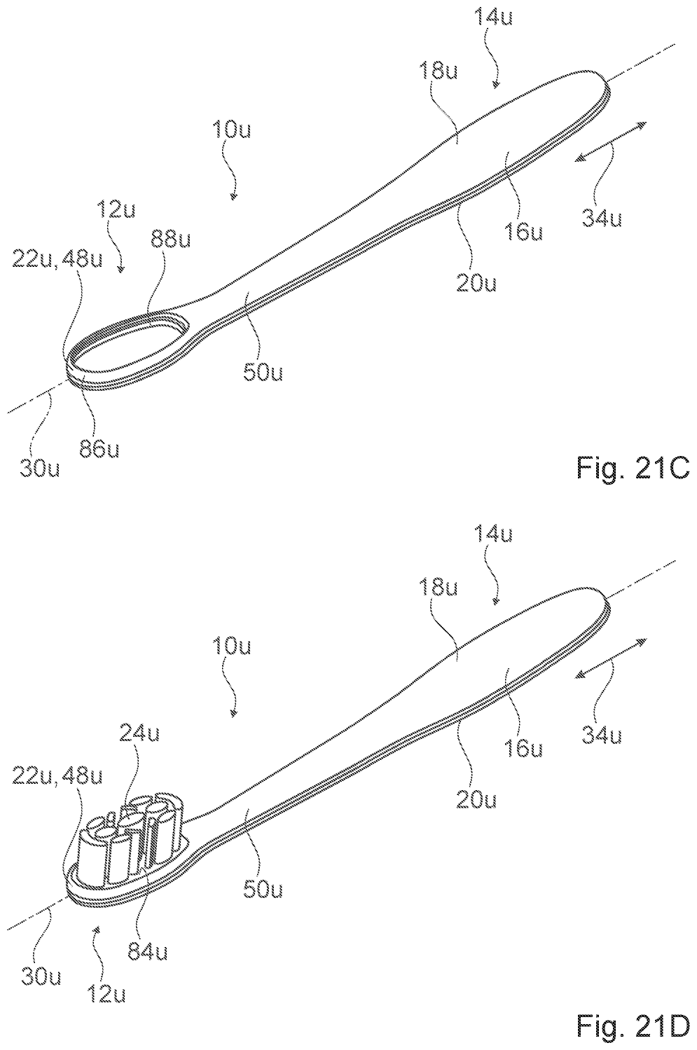

FIG. 19 shows a body care product 10s in a schematic perspective illustration. The body care product 10s is in the present case embodied as a toothpick.

The body care product 10s comprises an application unit 12s. The body care product 10s further comprises at least one handle unit 14s, which is connected to the application unit 12s.

The handle unit 14s is configured for a gripping by the operator. The handle unit 14s has a cylindrical, in particular solid-cylindrical, basic shape. The handle unit 14s further comprises a material handle body 16s. The material handle body 16s implements a material volume of the handle unit 14s. The material handle body 16s has a cylindrical shape. The material handle body 16s of the handle unit 14s is at least partially, in particular at least to a large extent, made of a paper material. The material handle body 16s is completely made of a paper material. The material handle body 16s of the handle unit 14s comprises at least one layer 18s of a paper material, which is realized in a rolled-up fashion. The layer 18s of a paper material is rolled around a defined rolling axis. The rolling axis extends parallel to, in particular coaxially with, a longitudinal axis 30s of the body care product 10s. The layer 18s is rolled up during a production of the material handle body 16s. In a final state, in particular after production, the layer 18s forms a roll, in particular a cylindrical roll. The layer 18s of the material handle body 16s is rolled to form a solid-cylindrical shape. The layer 18s of the material handle body 16s is connected in itself within the roll by gluing, welding and/or laminating. The paper material is rolled to form a solid-cylindrical roll. The material handle body 16s may in particular be rolled up straight or in a spiral shape.

The application unit 12s is implemented integrally with the handle unit 14s. The application unit 12s is realized by obliquely cutting off an end of the material handle body 16s from two sides. The one end of the material handle body 16s which forms the application unit 12s has at least partially the shape of a saddleback roof.

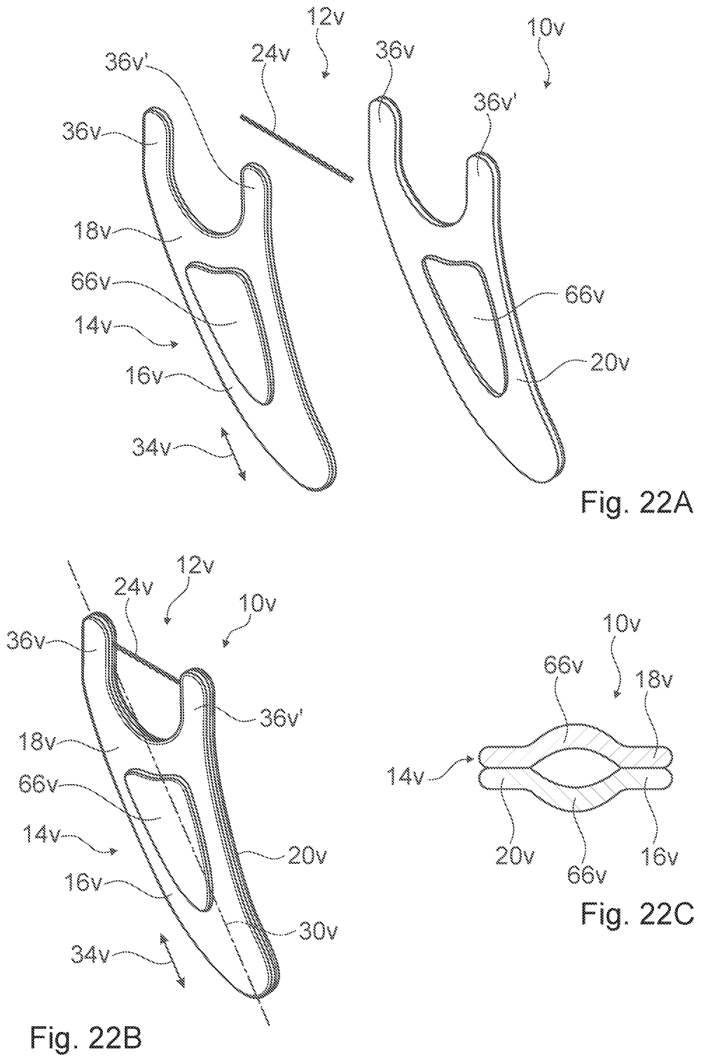

FIG. 20 shows a body care product 10t in a schematic perspective illustration. The body care product 10t is in the present case embodied as a toothpick.

The body care product 10t comprises an application unit 12t. The body care product 10t further comprises at least one handle unit 14t, which is connected to the application unit 12t.

The handle unit 14t is configured for a gripping by the operator. The handle unit 14t has a cylindrical, in particular solid-cylindrical, basic shape. The handle unit 14t further comprises a material handle body 16t. The material handle body 16t implements a material volume of the handle unit 14t. The material handle body 16t has a cylindrical shape. The material handle body 16t is at least partially, in particular at least completely, made of a paper material. The material handle body 16t is completely made of a paper material. The material handle body 16t comprises at least one layer 18t of a paper material, which is realized in a rolled-up fashion. The layer 18t of a paper material is rolled around a defined rolling axis. The rolling axis extends parallel to, in particular coaxially with, a longitudinal axis 30t of the body care product 10t. The layer 18t is rolled up during a production of the material handle body 16t. In a final state, in particular after production, the layer 18t forms a roll, in particular a cylindrical roll. The layer 18t of the material handle body 16t is rolled to form a solid-cylindrical shape. The layer 18t of the material handle body 16t is connected in itself by gluing, welding and/or laminating within the roll. The paper material is rolled to form a solid-cylindrical roll. The material handle body 16t may in particular be rolled up straight or in a spiral shape.

The application unit 12t is implemented integrally with the handle unit 14t. The application unit 12t is realized by cutting off an end of the material handle body 16t obliquely from two sides. The one end of the material handle body 16t which forms the application unit 12t has at least partially the shape of a saddleback roof. In addition, perforations 82t are provided in the region of the application unit 12t, which are intended to support cleaning.

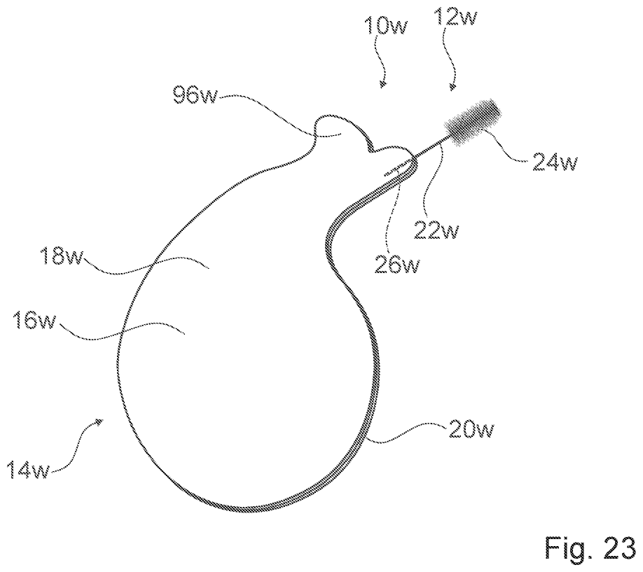

FIG. 21D shows a body care product 10u in a schematic perspective illustration. The body care product 10u is in the present case embodied as a toothbrush.

The body care product 10u comprises an application unit 12u. The body care product 10u further comprises at least one handle unit 14u, which is connected to the application unit 12u.

The application unit 12u comprises a head region 48u. The head region 48u forms a brush head of the body care product 10u. In the present case, the head region 48u is embodied as a toothbrush head. The application unit 12u comprises a bristle platelet 84u, which is accommodated in the head region 48u and comprises a plurality of bristle bundles (cf. FIG. 21A, 21B). By way of example, the bristle platelet 84u is realized as an AFT platelet. Any suitable bristles may be used as bristles. The bristle platelet 48u may be made of a paper material.

The head region 48u has a base body 86u. The base body 86u of the head region 48u is completely made of a paper material. The base body 86u is embodied in a one-part-implementation with a handle unit 14u of the body care product 10u. The base body 86u of the head region 48u serves for receiving the bristle platelet 84u. The base body 86u has an oval recess 88u for receiving the bristle platelet 84u (cf. FIG. 21C). The bristle platelet 84u may be just plugged into the recess 88u or may be glued, sealed, welded, etc. in the recess 88u.

The application unit 12u in the present case further comprises a neck region 50u, which in particular connects the head region 48u to the handle unit 14u. The neck region 50u forms a neck.

The handle unit 14u is configured for a gripping by the operator. The handle unit 14u further comprises a material handle body 16u. The material handle body 16u has a flat elongate basic shape. The material handle body 16u of the handle unit 14u is made at least partially, in particular at least to a large extent, of a paper material. The material handle body 16u is made completely of a paper material. The material handle body 16u of the handle unit 14u comprises at least two layers 18u, 20u of a paper material, which are connected to each other in a layered fashion. The material handle body 16u of the handle unit 14u exemplarily comprises precisely two layers 18u, 20u of a paper material. Preferably two to six, preferably two to four layers 18u, 20u are provided. Preferentially the at least two layers 18u, 20u are arranged above one another perpendicularly to a main extension plane of the layers 18u, 20u and are connected to one another. The layers 18u, 20u are realized substantially identically and in particular form opposite-situated outer sides of the body care product 10u. The layers 18u, 20u are connected to one another, for example, by gluing and/or pressing and/or welding and/or laminating. The layers 18u, 20u are in particular connected to one another during a production of the body care product 10u.

The base body 86u, the neck region 50u and the handle unit 14u are realized integrally.

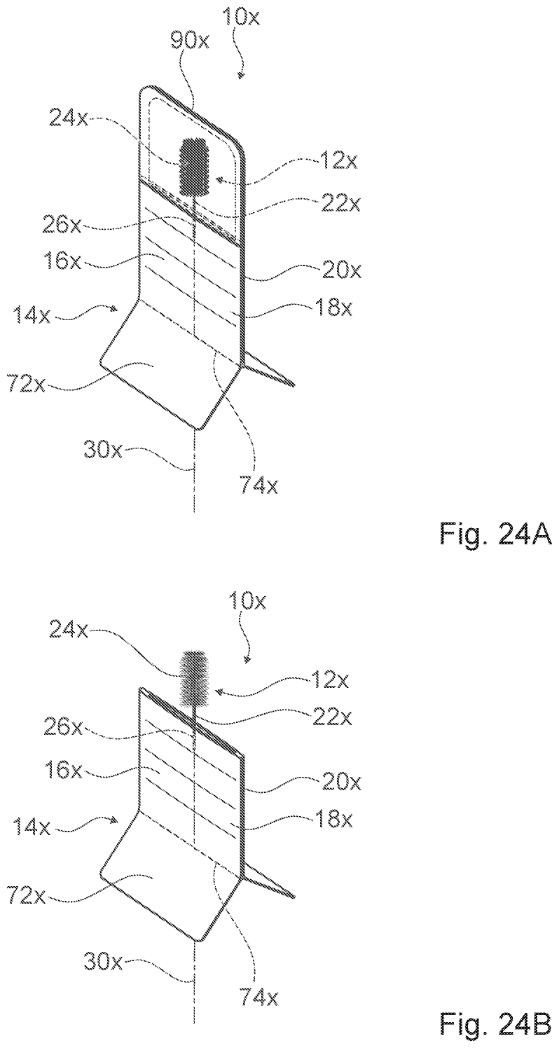

FIG. 22B with FIGS. 22A and 22C shows a body care product 10v which is embodied as a flosser. The body care product 10v comprises an application unit 12v. The body care product 10v further comprises at least one handle unit 14v, which is connected to the application unit 12v. The application unit 12v is arranged at the upper side of the body care product 10v. The application unit 12v constitutes an uppermost point of the body care product 10v. The application unit 12v comprises a cleaning element 24v, which is realized as a dental floss. The application unit 12v in particular consists of the cleaning element 24v.

The handle unit 14v is configured for a gripping by the operator. The handle unit 14v further comprises a material handle body 16v. The material handle body 16v implements a material volume of the handle unit 14v. The material handle body 16v has in a final state a three-dimensional basic shape, which is split into two arms 36v, 36v′ towards the application unit 12v. The arms 36v, 36v′ are configured to hold the cleaning element 24v on both sides. The material handle body 16v of the handle unit 14v is made at least partially, in particular at least to a large extent, of a paper material. The material handle body 16v is made completely of a paper material. The material handle body 16v of the handle unit 14v comprises at least two layers 18v, 20v of a paper material, which are connected to each other in a layered fashion. The material handle body 16v of the handle unit 14v exemplarily comprises precisely two layers 18v, 20v of a paper material. The layers 18v, 20v of the material handle body 16v are cut or punched.

Furthermore, the layers 18v, 20v of the material handle body 16v are deformed, preferably deep-drawn, in particular with the assistance of moistening, pressure and/or heat. The material handle body 16v has an oval deformation 66v, which is on both sides convex outwards. As a result, for example in particular a gripping geometry is created. It is in particular possible to provide a body care product 10v with a voluminous handle unit 14v.

FIG. 22C shows a section through the body care product of FIG. 22B in the region of the deformation 66v. It can be seen that the deformations 66v (also referred to as crownings) of the two layers 18v and 20v are oriented opposing each other so as to form a hollow space. The layers 18v, 20v are connected, thus forming a closed hollow space. The layers 18v, 20v in each case have an embossment realized as the oval deformation 66v.

The deformation 66v may be implemented in different ways. On the one hand it is possible that the individual layers are deformed before a connection, on the other hand the layers may be deformed as a whole after the connection. In the deformation process before a connection of the layers, it is crucial that the outer contour is correctly aligned with the deformation 66v. This is intended to achieve, if the layers are already punched out before a connection, that identical products are created without differences of the layers at the outer contour. On the other hand, this is intended to ensure, if the products are not punched out until after the deformation, that the deformation is always in the same place. In the case of a deformation after the connection, it must be considered that the deformation process requires greater effort due to the increased material thickness caused by a greater number of layers to be deformed. In the case presently shown in FIGS. 22A to 22C, the layers are deformed before a connection, otherwise it would not be possible to form a closed hollow space.

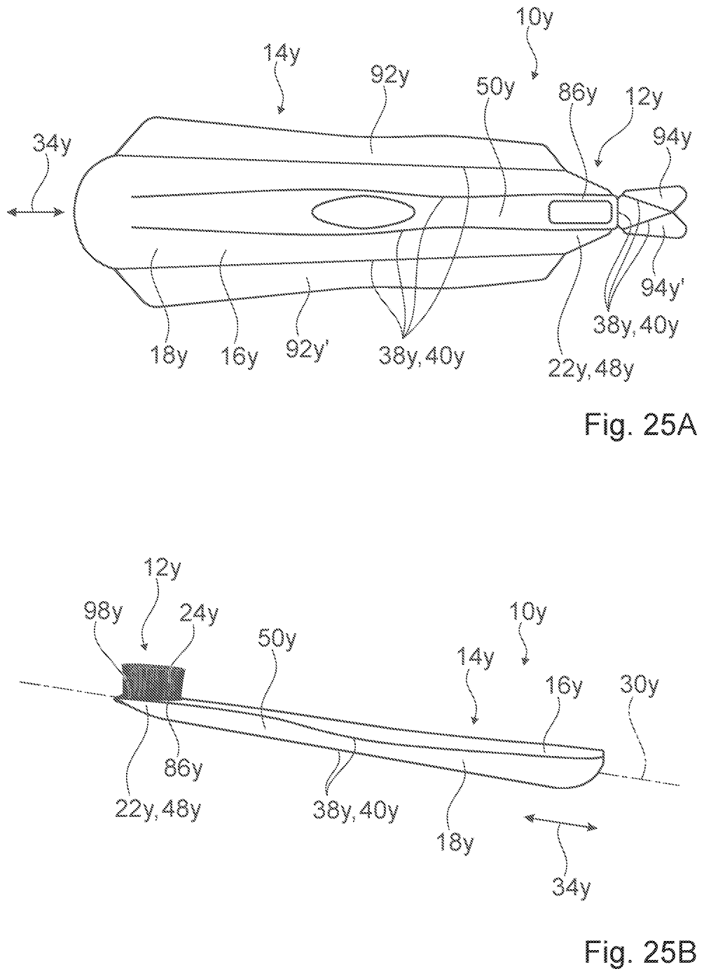

FIG. 23 shows a body care product 10w in a schematic illustration. The body care product 10w is in the present case embodied as an interdental brush. The body care product 10w comprises an application unit 12w. The body care product 10w further comprises at least one handle unit 14w, which is connected to the application unit 12w.

The application unit 12w is arranged at the upper side of the body care product 10w. The application unit 12w constitutes an uppermost point of the body care product 10w.

The handle unit 14w is configured for a gripping by the operator. The handle unit 14w further comprises a material handle body 16w. The material handle body 16w implements a material volume of the handle unit 14w. The material handle body 16w has a flat elongate basic shape. The material handle body 16w of the handle unit 14w is made at least partially, in particular at least to a large extent, of a paper material. The material handle body 16w is made completely of a paper material. The material handle body 16w of the handle unit 14w comprises at least two layers 18w, 20w of a paper material, which are connected to each other in a layered fashion. The material handle body 16w of the handle unit 14w exemplarily comprises precisely two layers 18w, 20w of a paper material. Preferably two to six, preferably two to four layers 18w, 20w are provided. Preferentially the at least two layers 18w, 20w are arranged above one another perpendicularly to a main extension plane of the layers 18w, 20w and are connected to one another. The layers 18w, 20w are realized substantially identically and in particular form opposite-situated outer sides of the body care product 10w. The layers 18w, 20w are connected to one another, for example, by gluing and/or pressing and/or welding and/or laminating. The layers 18w, 20w are in particular connected to one another during a production of the body care product 10w.

The application unit 12w further comprises a cleaning element carrier 22w and several cleaning elements 24w which are connected, in particular twisted in, with the cleaning element carrier 22w. The cleaning element carrier 22w is exemplarily implemented as a wire, the cleaning elements 24w being realized as bristles which are twisted into the wire.

For a connection to the handle unit 14w, the cleaning element carrier 22w is connected to the material handle body 16w in a connection section 26w, which faces away from the cleaning elements 24w. The connection section 26w is in particular realized as a wire end facing away from the cleaning elements 24w. In particular in a state when the body care product 10w has been completed, the connection section 26w of the cleaning element carrier 26w of the application unit 12w projects into the material handle body 16w and is enclosed by the material handle body 16w. The application unit 12w is connected to the material handle body 16w directly via the connection section 26w. The connection section 26w is arranged at the handle unit 14w between the at least two layers 18w, 20w of the material handle body 16w. Furthermore, for a partially form-fit connection to the handle unit 14w, the cleaning element carrier 22w of the application unit 12w is implemented so as to be curved (not visible).

The handle unit 14w comprises on a side facing towards the application unit 12w a cheek pull-back element 96w. The cheek pull-back element 96w is embodied integrally with the handle body 16w. The cheek pull-back element 96w is implemented by a projecting structure formed as a protruding nose. On the side of the nose that faces away from the application unit 12w, the skin of the cheek, respectively the corner of the mouth, can be pulled back by means of the cheek pull-back element 96w, such that the skin of the cheek does not get in the way when the body care product 10w, embodied as an interdental brush, is moved in the interdental spaces. There is also no friction between the body of the body care product 10w and the skin of the cheek when the body care product 10w is moved in the interdental spaces. The skin is pulled back by the projecting cheek pull-back element 96w and thus the elements will not rub against each other.

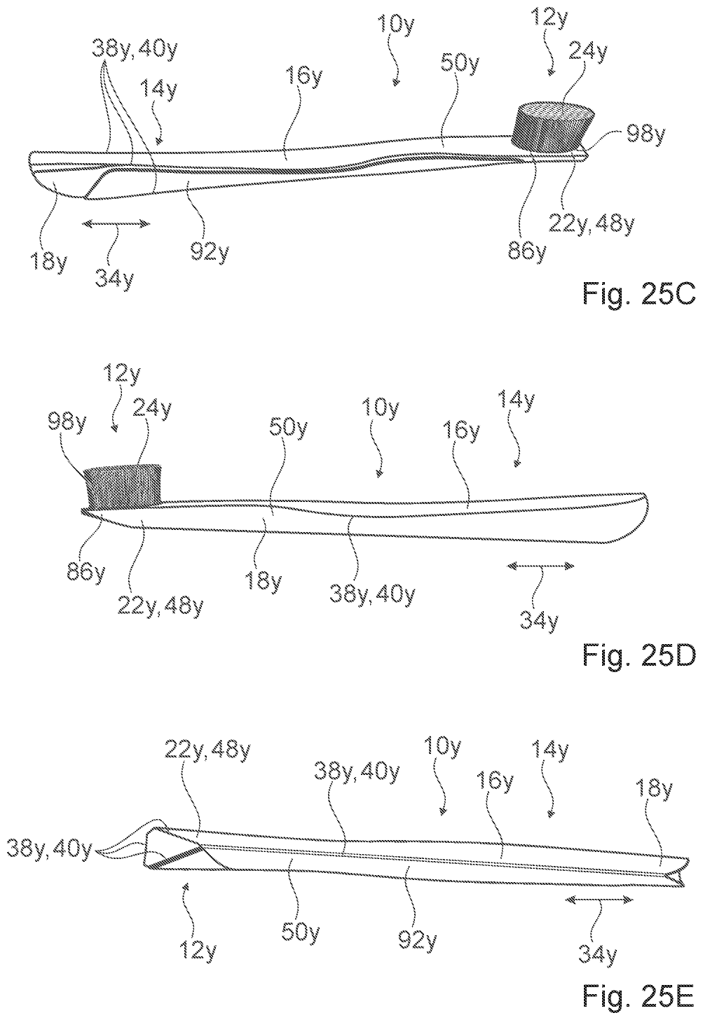

FIG. 24A shows an application unit 12x of a body care product 10x in a schematic illustration. The body care product 10x is in the present case embodied as an interdental brush.

The body care product 10x comprises the application unit 12x. The body care product 10x further comprises at least one handle unit 14x, which is connected to the application unit 12x. The body care product 10x moreover comprises a protective element 90x, which is connected to the handle unit 14x. The protective element 90x is realized by a covering cap. The protective element 90x is connected to the handle unit 14x releasably.

The application unit 12x is arranged at the upper side of the body care product 10x. The application unit 12x forms an uppermost point of the body care product 10x.

The handle unit 14x is configured for a gripping by an operator. The handle unit 14x further comprises a material handle body 16x. The material handle body 16x implements a material volume of the handle unit 14x. The material handle body 16x has a flat elongate basic shape. The material handle body 16x of the handle unit 14x is at least partially, in particular at least to a large extent, made of a paper material. The material handle body 16x is completely made of a paper material. The material handle body 16x of the handle unit 14x comprises at least two layers 18x, 20x of a paper material, which are connected to each other in a layered fashion. The material handle body 16x of the handle unit 14x exemplarily comprises precisely two layers 18x, 20x of a paper material. Preferably two to six, preferably two to four layers 18x, 20x are provided. Preferentially the at least two layers 18x, 20x are arranged above one another perpendicularly to a main extension direction of the layers 18x, 20x and are connected to one another. The layers 18x, 20x are realized substantially identically to one another and in particular form opposite-situated outer sides of the body care product 10x. The layers 18x, 20x are connected to one another, for example, by gluing and/or pressing and/or welding and/or laminating. The layers 18x, 20x are in particular connected to one another during a production of the body care product 10x.

The application unit 12x further comprises a cleaning element carrier 22x and several cleaning elements 24x, which are connected, in particular twisted-in, with the cleaning element carrier 22x. The cleaning element carrier 22x is exemplarily implemented by a wire, the cleaning elements 24x being bristles which are twisted into the wire.

For a connection to the handle unit 14x, the cleaning element carrier 22x is connected to the material handle body 16x in a connection section 26x that faces away from the cleaning elements 24x. The connection section 26x is in particular realized as a wire end facing away from the cleaning elements 24x. In particular in a state when the body care product 10x has been completed, the connection section 26x of the cleaning element carrier 22x of the application unit 12x projects into the material handle body 16x and is enclosed by the material handle body 16x. The application unit 12x is connected directly to the material handle body 16x via the connection section 26x. The connection section 26x is arranged at the handle unit 14x between the at least two layers 18x, 20x of the material handle body 16x. Furthermore, for a partially form-fit connection to the handle unit 14x, the cleaning element carrier 22x of the application unit 12x is realized so as to be bent (not visible) in the connection section 26x in order to increase a holding force.

The handle unit 14x furthermore forms a base foot 72x. The two layers 18x, 20x are for this purpose realized so as to be unconnected in a lower section that faces away from the application unit 12x. The base foot 72x is provided by folding the two layers 18x, 20x apart in the lower section. In particular, folding edges 74x are provided at which the layers 18x, 20x can be folded away from each other in the lower section (cf. FIG. 24B). This may in addition be used for a complete separation of the layers 18x, 20x for a material-side separation of the body care product 10x from the paper material for disposal purposes. Thus the paper material may be disposed into the paper collection and the application unit 12x may be disposed separately into the household waste.

The protective element 90x is made at least partially, in particular at least to a large extent, of a paper material. The protective element 90x is made completely of a paper material. The protective element 90x comprises at least two layers 18x, 20x of a paper material, which are connected to each other in a layered fashion. The material handle body 16x of the handle unit 14x exemplarily comprises precisely two layers 18x, 20x which form, in addition to the handle body 16x, the protective element 90x, respectively the covering cap. The protective element 90x forms a receiving region between the two layers 18x and 20x, which is configured to receive the application unit 12x in a non-use state of the body care product 10x. The receiving region may be implemented as a hollow space for a protection of the application unit 12x. The receiving region is in particular produced by deformations of the layers 18x, 20x. For this the protective element 90x is connected to the handle unit 14x, at an upper side of the handle unit 14x, so as to be separable, in particular via a perforation. The protective element 90x and the handle unit 14x are in particular produced from one part. The protective element 90x is in particular configured to be separated from the handle unit 14x before a first usage. It is in particular conceivable that the protective element 90x serves as a sales protection allowing to sell the body care product 10x without additional packaging.

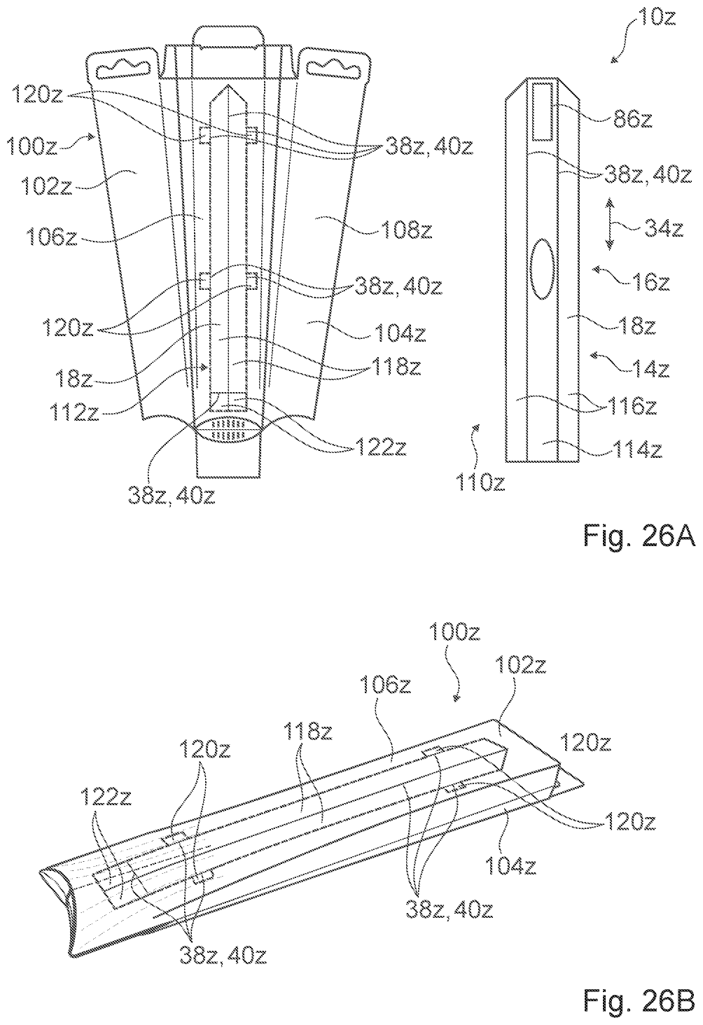

FIG. 25B shows a body care product 10y in a schematic perspective illustration. The body care product 10y is in the present case embodied as a toothbrush.

The body care product 10y comprises an application unit 12y. The body care product 10y further comprises at least one handle unit 14y, which is connected to the application unit 12y.

The application unit 12y has a head region 48y. The head region 48y forms a brush head of the body care product 10y. In the present case, the head region 48y is embodied as a toothbrush head. The application unit 12y comprises a bristle-furnished element 98y, which is accommodated in the head region 48y and comprises a plurality of bristle bundles. The bristle-furnished element 98y is implemented of bristles which are connected but, differently than the bristle platelets shown in the other figures, have no bristle platelet as such. Any suitable bristles may be used as bristles. The head region 48y may comprise an injection-molded bristle field.

The head region 48y has a base body 86y. The base body 86y of the head region 48y is completely made of a paper material. The base body 86y is realized in a one-part implementation with a handle unit 14y of the body care product 10y. The base body 86y of the head region 48y serves for accommodating the bristle-furnished element 98y. The base body 86y comprises a surface which the bristle-furnished element 98y may be applied onto by means of a gluing connection, by welding, etc.

In the present case, the application unit 12y further comprises a neck region 50y, which in particular connects the head region 48y to the handle unit 14y. The neck region 50y forms a neck.

The handle unit 14y is configured for a gripping by the operator. The handle unit 14y further comprises a material handle body 16y. The material handle body 16y implements a material volume of the handle unit 14y. The material handle body 16y has an elongate basic shape. The material handle body 16y of the handle unit 14y is made at least partially, in particular at least to a large extent, of a paper material. The material handle body 16y is made completely of a paper material. The material handle body 16y of the handle unit 14y comprises at least one layer 18y of a paper material. The material handle body 16y of the handle unit 14y exemplarily comprises precisely one layer 18y of a paper material. The material handle body 16y of the handle unit 14y comprises at least one layer 18y of a paper material, which is realized so as to be at least partially folded in a usage state.

The layer 18y has several folding axes 38y. In the folding axes 38y the layer 18y in each case has a respective folding edge 40y. In a usage state, the layer 38y is folded at the folding edges 40y around the respective folding axis 38y. The layer 18y is realized so as to be perforated, fluted and/or embossed in the folding edges 40y in order to enable defined folding. Four first folding axes 38y of the layer 18y extend substantially parallel to a main extension direction 34y of the body care product 10y, wherein two of the four first folding axes 38y do not extend over the complete extension of the body care product 10y, thus realizing a rounding, respectively a rounded transition, of the handle body 16y in a lower end region. The two further first folding axes 38y of the four first folding axes 38y are in each case adjoined by a gluing tab 92y, 92y′ for closing the handle body 16y on a rear side. Three further folding axes 38y are arranged in the head region 48y of the body care product 10y. A first further folding axis 38y extends perpendicularly to the main extension direction 34y of the body care product 10y and includes a triangle with the two further folding axes 38y. The two further folding axes 38y are in each case adjoined by a small gluing tab 94y, 94y′ for closing the head region 48y on a front side.

The material handle body 16y may herein in particular be folded from paper sheets or from a reel.

The base body 86y, the neck region 50y and the handle unit 14y are embodied integrally. By the forming and connection of the layers a body care product 10y is created, which is closed at an upper end in the head region 48y while designed to be open at a lower end of the handle unit 14y.

Alternatively, the bristle-furnished end 98y may be replaced by a bristle platelet.

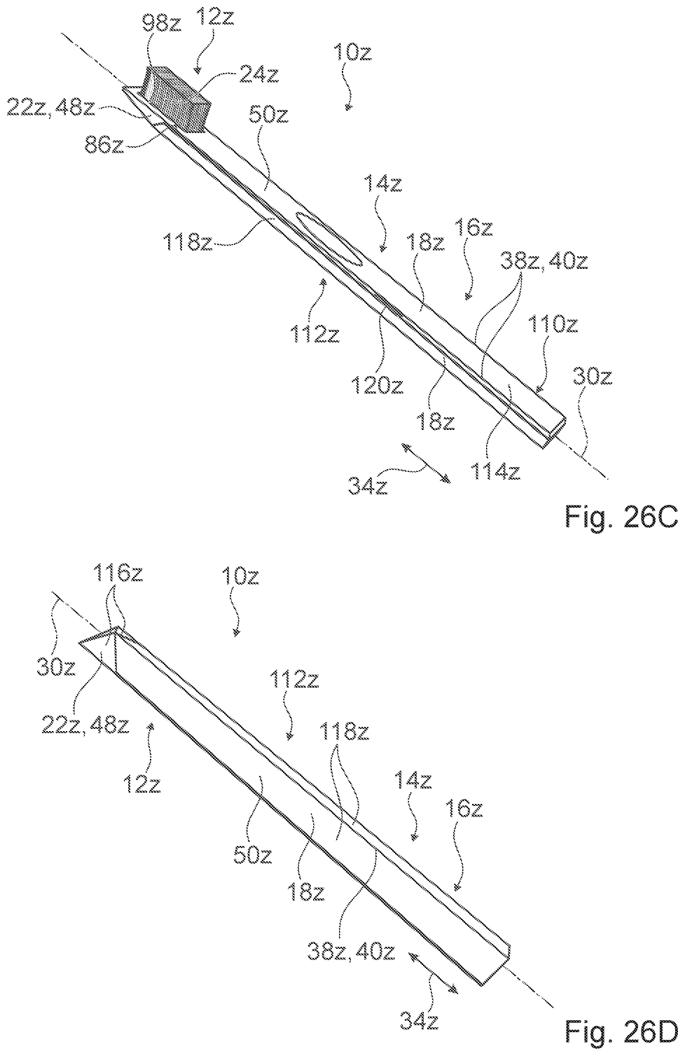

FIG. 26A shows a sales unit with a packaging 100z and with a body care product 10z, which is embodied as a toothbrush, in a non-folded state.

The packaging 100z comprises a packaging base body 102z. The packaging base body 102z is configured to form a substantially closed receiving region for the body care product 10z. The packaging base body 102z is formed from a blank. The packaging base body 102z is realized as a layer 104z of a paper material. The packaging 100z consists of the packaging base body 102z. The packaging base body 102z is embodied as a folded and glued box, which can be seen in FIG. 26B.

The receiving region of the brush product packaging device 100z has in a lower region, perpendicularly to a main extension direction, a cross section that differs substantially from a cross section in an upper region. The receiving region has in the lower region, perpendicularly to the main extension direction, a lentiform cross section. Furthermore, the receiving region has in an upper region, perpendicularly to the main extension direction, a rectangular cross section. The packaging base body 102z has a decreasing height profile from the front side to the rear side and from the top to the bottom, thus presenting a wedge shape and in addition a crowning. The packaging base body 102z has a substantially rectangular front side 106z and a substantially rectangular rear side 108z.

FIG. 26B shows the packaging 100z in a folded and closed state.

The body care product 10z comprises an application unit 12z. The body care product 10z further comprises at least one handle unit 14z, which is connected to the application unit 12z.

The application unit 12z has a head region 48z. The head region 48z forms a brush head of the body care product 10z. In the present case the head region 48z is embodied as a toothbrush head. The application unit 12z comprises a bristle-furnished element 98z, which is accommodated in the head region 48z and comprises a plurality of bristle bundles. The bristle-furnished element 98z is implemented by bristles which are connected but, differently than the bristle platelets shown in other figures, have no bristle platelet as such. Any suitable bristles may be used as bristles.

The head region 48z has a base body 86z. The base body 86z of the head region 48z is completely made of a paper material. The base body 86z is realized in a one-part implementation with the handle unit 14z of the body care product 10z. The base body 86z of the head region 48z serves for accommodating the bristle-furnished element 98z. The base body 86z comprises a surface on which the bristle-furnished element 98z can be applied by a glue connection, by welding, etc.

In the present case the application unit 12z comprises a neck region 50z, which in particular connects the head region 48z to the handle unit 14z. The neck region 50z forms a neck.

The handle unit 14z is configured for a gripping by the operator. The handle unit 14z further comprises a material handle body 16z. The material handle body 16z implements a material volume of the handle unit 14z. The material handle body 16z has an elongate basic shape. The material handle body 16z of the handle unit 12z is made at least partially, in particular to a large extent, of a paper material. The material handle body 16z is made completely of a paper material. The material handle body 16z of the handle unit 14z comprises at least one layer 18z of a paper material. The material handle body 16z is realized in a multi-part implementation. The material handle body 16z has a front part 110z, which forms a front side of the handle unit 14z, and a rear part 112z, which forms a rear side of the handle unit 14z. The front part 110z and the rear part 112z exemplarily in each case comprise precisely one layer 18z of paper material. The front part 110z and the rear part 112z exemplarily in each case comprise a layer of paper material which is in a usage state realized so as to be at least partially folded. The front part 110z and the rear part 112z may comprise several layers.

The layer 18z of the front part 110z comprises several folding axes 38z. The layer 18z of the front part 110z has respectively one folding edge 40z in the folding axes 38z. In a usage state, the layer 18z of the front part 110z is folded at the folding edges 40z around the respective folding axis 38z. The layer 18z of the front part 110z is implemented so as to be perforated, fluted and/or embossed in the folding edges 40z so as to enable defined folding. The front part 110z has precisely two folding axes 38z in the layer 18z, which extend substantially parallel to a main extension direction 34z of the body care product 10z. By means of the folding axes 38z, the front part 110z is foldable to form a triangular cross-section shape. The front part 110z has a front side 114z and two side wings 116z, the side wings 116z being connected to the front side 114z via the folding edges 40z of the folding axes 38z. The front side 114z of the front part 110z of the material handle body 16z merges directly into the application unit 12z and also forms the base body 86z of the application unit 12z.

The layer 18z of the rear part 112z is realized integrally with the packaging 100z. The material handle body 16z of the handle unit 14z of the body care product 10z comprises a layer 18z of paper material, the layer 18z being realized at least partially integrally with the packaging 100z. The layer 18z of the rear part 112z is realized integrally with the front side 106z of the packaging 100z as the layers of the packaging 100z are not connected on the front side 106z and lie above one another. The rear part 112z is realized as a portion of the packaging 100z which can, after unpacking, be separated out of the packaging 100z by an operator by means of a perforation in order to create the body care product 10z. The shape of the rear part 112z is introduced in the front side 106z of the packaging 100z by a perforation. In a sales state, the body care product 10z is not fully assembled.

The rear part 112z comprises several folding axes 38z. In the folding axes 38z the layer 18z of the rear part 112z in each case has a folding edge 40z. The rear part 112z is in a usage state folded at the folding edges 40z around the respective folding axis 38z. The rear part 112z is perforated, fluted and/or embossed in the folding edges 40z so as to enable defined folding. The rear part 112z comprises precisely three folding axes 38z in the layer 18z which extend substantially parallel to a main extension direction 34z of the body care product 10z. The rear part 112z furthermore comprises precisely one folding axis 38z in the layer 18z which extends substantially perpendicularly to a main extension direction 34z of the body care product 10z. By means of the folding axes 38z, the rear part 112z can be folded to form a V-shaped cross-section shape. The rear part 112z has two rear sides 118z, four latching wings 120z and two folding wings 122z, the latching wings 120z and the folding wings 122z being connected to the rear sides 118z via the folding edges 40z of the folding axes 38z. The rear sides 118z are in a folded state of the body care product 10z configured to adjoin the side wings 116z of the front part 110z and to at least partially cover the side wings 116z. For a connection to the front part 110z, the latching wings 120z are configured to be slid into slits (not visible) in the front part 110z. For a further fixing of the rear part 112z, the folding wings 122z are configured to be folded behind the side wings 116z and, depending on a respective implementation, to close the handle body 16z.

The material handle body 16z of the handle unit 14z comprises at least one layer 18z of a paper material, which is realized so as to be at least partially folded, the handle body 16z having a stowage position, in which an inner face of the layer 18z is oriented outwards, and an operational position, in which an outer face of the layer 18z is oriented outwards. An outer side of the rear part 112z is in a state when connected to the packaging 100z oriented inwards. The layer 18z of the rear part 112z has an inner face, which is in the stowage position, in particular in a packaged state of the body care product 10z, oriented outwards, and has an outer face, which is in the operational position, in which the body care product 10z is plugged together, oriented outwards.

The base body 86z, the neck region 50z and the handle unit 14z are realized substantially integrally. By the forming and connecting of the front part 110z and the rear part 112z, a body care product 10z is created.

The material handle body 16z of the handle unit 14z may herein comprise at least one layer 18z of a paper material, which is at least partially folded and is realized so as to be connected by at least one plug connection, wherein at least one first folded plug connection forms a thumb grip of the material handle body 16z.

The material handle body 16z may herein in particular be folded from paper sheets or from a reel.

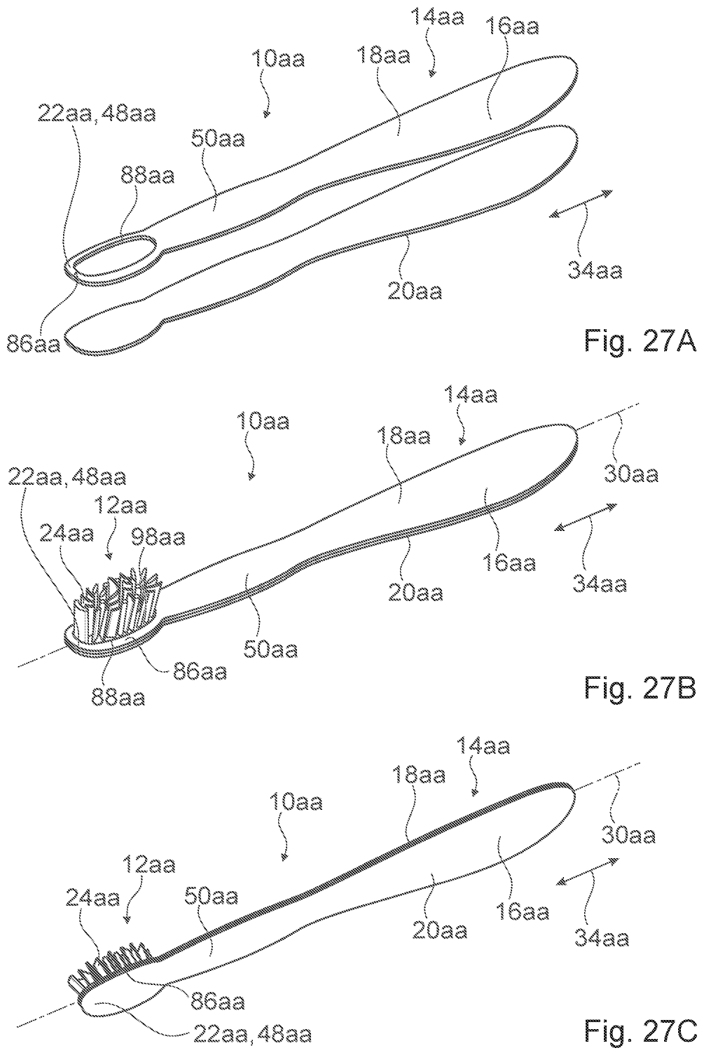

FIG. 27B shows a body care product 10aa in a schematic perspective view from the front. The body care product 10aa is in the present case embodied as a toothbrush.

The body care product 10aa comprises an application unit 12aa. The body care product 10aa further comprises at least one handle unit 14aa, which is connected to the application unit 12aa.

The application unit 12aa comprises a head region 48aa. The head region 48aa forms a brush head of the body care product 10aa. In the present case, the head region 48aa is embodied as a toothbrush head. The application unit 12aa comprises a bristle-furnished element 98aa, which is accommodated in the head region 48aa and comprises a plurality of bristle bundles. The bristle-furnished element 98aa is implemented of bristles which are connected. Any suitable bristles may be used as bristles. Herein the head region 48aa may comprise an injection-molded bristle field or the bristle field is produced in an AFT-like manner without a platelet.

The head region 48aa has a base body 86aa. The base body 86aa of the head region 48aa is completely made of a paper material. The base body 86aa is realized in a one-part implementation with a handle unit 14aa of the body care product 10aa. The base body 86aa of the head region 48aa serves for accommodating the bristle-furnished element 98aa. The base body 86aa has an oval recess 88aa shaped as a through hole for accommodating the bristle-furnished element 98aa. The bristle-furnished element 98aa is guided through the oval recess 88aa from the rear side. The bristle-furnished element 98aa may be just plugged into the recess 88a or may be glued, sealed, welded etc. into the recess 88aa. The bristle-furnished element 98aa in particular has a large jutting edge sidewise. In a production, the bristle-furnished element 98aa is in particular laid into the recess 88aa, in particular into a first layer 18aa from the rear, wherein the jutting edge prevents the bristle-furnished element 98aa from falling through and helps with holding the bristle-furnished element 98aa after a connection of the layers 18aa, 20aa.

In the present case the application unit 12aa further comprises a neck region 50aa, which in particular connects the head region 48aa to the handle unit 14aa. The neck region 50aa forms a neck.

The handle unit 14aa is configured for a gripping by the operator. The handle unit 14aa further comprises a material handle body 16aa. The material handle body 16aa implements a material volume of the handle unit 14aa. The material handle body 16aa has a flat elongate basic shape, wherein the material handle body 16aa is formed in a curved shape in a plane perpendicular to a main extension direction 34aa. In the head region 48aa the curvature must be adapted to the bristle-furnished element 98aa in order to allow an anchoring. The material handle body 16aa of the handle unit 14aa is made at least partially, in particular at least to a large extent, of a paper material. The material handle body 16aa is made completely of a paper material. The material handle body 16aa of the handle unit 14aa comprises at least two layers 18aa, 20aa of a paper material, which are connected to each other in a layered fashion. The material handle body 16aa of the handle unit 14aa exemplarily comprises precisely two layers 18aa, 20aa of a paper material. Preferably two to six, preferably two to four layers 18aa, 20aa are provided. The layers 18aa, 20aa in particular consist of several plies. Preferentially the at least two layers 18aa, 20aa are arranged above one another perpendicularly to a main extension plane of the layers 18aa, 20aa and are connected to one another. The layers 18aa, 20aa are realized substantially identically and in particular form opposite-situated outer sides of the body care product 10aa. The layers 18aa, 20aa are connected to one another, for example, by gluing and/or pressing and/or welding and/or laminating. The layers 18aa, 20aa are in particular connected to one another during a production of the body care product 10aa.

The layers 18aa, 20aa extend over the base body 86aa, the neck region 50aa and the handle unit 14aa. The oval recess 88aa is introduced only into the first layer 18aa whereas the second layer 20aa is realized so as to be continuous.

In a production the bristle-furnished element 98aa is provided without a bristle platelet. In the production a melt blanket is formed, said melt blanket is realized larger in its longitudinal and transversal dimensions for the future anchoring/connection with the layers 18aa, 20aa. The jutting portion of the melt blanket is later on embedded between the layers 18aa, 20aa of paper material. Alternatively, the bristle-furnished element 98aa may also be implemented as an injection-molded element with injection-molded bristles, which also should have a jutting portion.

Furthermore, the two layers 18aa, 20aa of the handle unit 14aa are prepared: on the one hand the first layer 18aa, which will form the front side of the body care product 10aa later on. This layer 18aa may be designed with different curvatures so as to give stability to the handle unit 14aa later on. Beyond this, in the head region of this layer 18aa the recess 88aa is implemented as a through hole which is configured to accommodate the bristle-furnished element 98aa later on, respectively the bristle-furnished element 98aa will be guided through the through hole.

The second layer 20aa that is prepared has substantially the same outer shape as the first layer 18aa but is not provided with a recess. In the head region 48aa of the body care product 10aa, this second layer 20aa is implemented in such a way that it adjoins the melt blanket of the bristle-furnished element 98aa in the mounted state.

The head region 48aa of the two layers 18aa, 20aa may be realized so as to be planar or slightly curved or curved only at the edges; a limiting factor being that the bristle-furnished element 98aa must be held, respectively fixed. In the further regions the second layer 20aa may have the same shape as the first layer 18aa, such that they directly adjoin each other. Or the second layer 20aa may have a curvature inverse to the first layer 18aa so as to form a hollow space between the layers 18aa, 20aa in the mounted state. Herein, in the region of the hollow space the connection of the layers 18aa, 20aa will only be possible in a peripheral region of the hollow space.

After providing the layers 18aa, 20aa, a bringing-together of the elements will take place. The bristle-furnished element 98aa is guided through the recess 88aa of the first layer 18aa; then the second layer 20aa is mounted onto the first layer from the rear. For a connection of the layers 18aa, 20aa, the different well-known connection techniques may be applied. The bristle-furnished element 98aa may in particular be glued onto the layer 20aa, possible also onto the layer 18aa, such that it cannot be torn out through the recess.

Alternatively or additionally, the material base body 16aa may comprise a hollow space, for example in a rearward handle region.

The descriptions given for specific figures are of course also transferable to other figures which present the same or similar implementations and in which said implementations are not described in such detail.

### REFERENCE NUMERALS

10 body care product
12 application unit
14 handle unit
16 handle body
18 layer
20 layer
22 cleaning element carrier
24 cleaning element
26 connection section
28 plug connection unit
30 longitudinal axis
34 main extension direction
36 arm
36′ arm
38 folding axis
40 folding edge
42 sub-layer
42′ sub-layer
44 tension tab
44′ tension tab
46 thread
48 head region
50 neck region
52 base body
54 sub-element
54′ sub-element
56 sub-element
56′ sub-element
56″ sub-element
56— sub-element
58 form-fitting recess
58′ form-fitting recess
60 plug connection element
62 plug connection element
64 core projection
66 deformation
68 sub-layer
68′ sub-layer
68″ sub-layer
70 printing
72 base foot
74 folding edge
76 projection
78 arrow
80 layer
82 perforation
84 bristle platelet
86 base body
88 recess
90 protective element
92 gluing tab
94 gluing tab
96 cheek pull-back element
98 element
100 packaging
102 packaging base body
104 layer
106 front side
108 rear side
110 front part
112 rear part
114 front side
116 side wing
118 rear side
120 latching wing
122 folding wing

1. A body care product with at least one application unit and with at least one handle unit that is connected with the application unit and comprises at least one material handle body,
wherein
the material handle body of the handle unit and/or the application unit are/is made at least partially of a paper material.

2. The body care product according to claim 1,
wherein
the application unit comprises at least one cleaning element carrier and several cleaning elements which are connected with the cleaning element carrier, wherein for a connection to the handle unit the cleaning element carrier is connected to the material handle body in a connection section facing away from the cleaning elements.

3. The body care product according to claim 2,
wherein
the connection section of the cleaning element carrier of the application unit projects into the material handle body and is enclosed by the material handle body.

4. The body care product according to claim 2,
wherein
the at least one cleaning element carrier of the application unit is implemented so as to be rolled into at least one layer of the material handle body in a connection section that faces away from the cleaning elements.

5. The body care product according to claim 2,
wherein
the connection section of the cleaning element carrier has an angled structure for an anchoring in the handle body.

6. The body care product according to claim 2, wherein
the at least one cleaning element carrier of the application unit is realized so as to be bent in the connection section for an at least partially form-fit connection to the handle unit.

7. The body care product according to claim 2, wherein
the at least one cleaning element carrier of the application unit is connected to the material handle body at least partially by substance-to-substance bond.

8. The body care product according to claim 1,
wherein
the material handle body of the handle unit comprises at least one layer of a paper material which is realized in a rolled-up fashion.

9. The body care product according to claim 8,
wherein
the at least one layer of the material handle body is rolled in a cylindrical shape.

10. The body care product according to claim 1,
wherein
the material handle body of the handle unit comprises at least one layer of a paper material, said layer having an embossment and/or a deformation.

11. The body care product according to claim 1,
wherein
the material handle body of the handle unit comprises at least two layers of a paper material, which are connected to each other in a layered fashion.

12. The body care product according to claim 1,
wherein
the material handle body of the handle unit comprises at least one layer of a paper material, which is realized so as to be at least partially folded.

13. The body care product according to claim 11,
wherein
the handle body comprises tension tabs, the application unit being anchored at the tension tabs of the material handle body.

14. The body care product according to claim 13,
wherein
the cleaning element of the application unit is guided around the tension tabs.

15. The body care product according to claim 11,
wherein
the application unit is in at least one connection section glued and/or laminated in the material handle body in between the at least two layers.

16. The body care product according to claim 11,
wherein
the application unit displaces in at least one connection section at least one of the layers of flat paper material.

17. The body care product according to claim 1,
wherein
the application unit has at least one cleaning element comprising a paper material.

18. The body care product according to claim 16,
wherein
the at least one cleaning element is embodied at least partially as an interdental cleaning paper.

19. The body care product according to claim 11,
wherein
the application unit is arranged at the handle unit between the at least two layers of the material handle body.

20. The body care product according to claim 1,
wherein
the application unit comprises at least one cleaning element implemented as a thread, which is in at least one connection section realized so as to be partly melted.

21. The body care product according to claim 1,
wherein
the application unit comprises at least one cleaning element which is connected to the material handle body by means of two parallel fixings.

22. The body care product according to claim 21,
wherein
the cleaning element is connected to the material handle body by gluing and by partial melting.

23. The body care product according to claim 1,
comprising
a protective element, which is connected with the handle unit and is releasably connected with the handle unit.

24. The body care product according to claim 1,
comprising
a plug connection unit configured for a releasable plug connection of the application unit to the handle unit.

25. The body care product according to claim 1,
wherein
the material handle body of the handle unit comprises several sub-elements which are configured to be plugged together.

26. The body care product according to claim 1,
wherein
the at least one application unit comprises a head region which forms a brush head.

27. The body care product according to claim 26,
wherein
a base body of the head region is completely made of a hard component and forms a bristle carrier.

28. The body care product according to claim 26,
comprising
a bristle platelet, which is accommodated in the head region and comprises a plurality of bristle bundles.

29. The body care product according to claim 28,
wherein
the bristle platelet is produced from a paper material.

30. The body care product according to claim 1,
wherein
the material handle body of the handle unit at least one layer of paper material, which is realized so as to be at least partially folded, the handle body having a stowage position, in which an inner face of the layer is oriented outwards, and an operational position, in which an outer face of the layer is oriented outwards.

31. The body care product according to claim 26,
wherein
the head region comprises an injection-molded bristle field.

32. The body care product according to claim 1,
wherein
the material handle body of the handle unit at least one layer of paper material, which is realized so as to be at least partially folded and to be connected by means of at least one plug connection, wherein at least one first folded plug connection forms a thumb grip of the material handle body.

33. The body care product according to claim 1,
wherein
the paper material has a water-repellent coating and/or impregnation.

34. The body care product according to claim 1,
wherein
the paper material is realized so as to be varnished and/or printed.

35. A system with several body care products according to claim 1, which in each case have a material handle body, the material handle bodies of the body care products being connected to each other in such a way that they are separable at perforations.

36. A sales unit with at least one packaging which is at least partially made of a paper material, and with at least one body care product according to claim 1, wherein a material handle body of the handle unit of the body care product comprises at least one layer of a paper material, wherein the layer is implemented at least partially integrally with the packaging.

37. A method for producing a body care product according to claim 1.

38. The method according to claim 37,
wherein
the material handle body of the handle unit is rolled onto at least one layer of paper material.

39. The method according to claim 38,
wherein
the application unit is in at least one connection section (26a) rolled into the material handle body.

40. The method according to claim 37,
wherein
the material handle body is layered from at least two interconnected layers of a paper material.

41. The method according to claim 40,
wherein
the application unit is in at least one connection section of the at least two layers glued and/or laminated into the material handle body.
